(19) United States
(12) Patent Application Publication
(10) Pub. No.: US 2009/0203750 A1
(43) Pub. Date: Aug. 13, 2009

(54) 5-HT2C Receptor Agonists as Anorectic Agents

(76) Inventors: Alan Kozikowski, Chicago, IL (US); Toru Kurome, Hyogo (JP); Vincent Setola, Ocean Pines, MD (US); Bryan Roth, Durham, NC (US)

Correspondence Address: GREENLEE WINNER AND SULLIVAN P C, 4875 PEARL EAST CIRCLE, SUITE 200, BOULDER, CO, 80301, US

(21) Appl. No.: 12/064,447

(22) PCT Filed: Aug. 24, 2006
(86) PCT No.: PCT/US06/33251

§ 371 Date: Jul. 29, 2008

Related U.S. Application Data
(60) Provisional application No. 60/711,078, filed on Aug. 24, 2005.

(51) Int. Cl. A61K 31/135; A61K 31/4409; C07C 211/17; C07C 211/19; C07D 213/36; A61P 3/00

(52) U.S. Cl. 514/357; 514/646; 514/657; 564/305; 564/428; 546/329

(57) ABSTRACT

This invention relates to compounds which modulate receptors of the 5-HT2 family of receptors, and particularly to compounds which modulate 5-HT2C receptors. Compounds of the invention include agonists and selective agonists for the 5-HT2C receptor Compounds of the invention include selective agonists for the 5-HT2C receptor which exhibit significantly less or no agonist activity on the 5-HT2A receptor and/or the 5-HT2B receptor. Compounds of this invention are those of Formula I and pharmaceutically acceptable salts, esters and solvates (including hydrates) wherein variables are defined in the specification hereof.

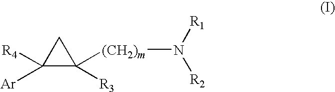

### CROSS-REFERENCE TO RELATED APPLICATIONS

This application claims priority to U.S. provisional application Ser. No. 60/711,078, filed Aug. 24, 2005 which is incorporated by reference in its entirety herein.

### STATEMENT REGARDING GOVERNMENT FUNDING

Not Applicable

### BACKGROUND OF THE INVENTION

Obesity and obesity-related health disorders, including hyperlipidaemia and its sequellae (e.g., coronary artery disease, CAD) and non-insulin dependent diabetes (NIDD), are among the greatest current health problems in the United States. Studies using reverse genetics and various genomic and linkage approaches have identified a number of molecular targets for treating obesity. These include leptin and its receptors, the melanocortin receptors, various neurotransmitter transporters and a variety of enzymes and peptides. To date, however, none of these molecular targets have yielded effective treatments for obesity.

Effective treatments for obesity have traditionally targeted monoamine transporters, particularly norepinephrine and serotonin (5-hydroxytryptamine or 5-HT). A current example of a modestly effective anorectic agent is sibutramine, which blocks serotonin and norepinephrine uptake. Before sibutramine, a first-line anti-obesity treatment was the use of racemic fenfluramine or its D-isomer (dexfenfluramine; Redux™, Servier) alone or in combination with phentermine (e.g., Fen/Phen). Fen/Phen and dexfen-based treatments were quite effective in inducing and sustaining weight loss, though not without significant health risks, including cardiac valvulopathy, rare cases of pulmonary hypertension and neurotoxicity to serotonergic neurones. Raceamic fenfluramine and D-fenfluramine were withdrawn from the U.S. market because their prolonged use was associated with valvular heart disease.

Initially, it was thought that fenfluramine, which is an amphetamine derivative, induced its anorectic activity via enhancement of serotonergic neurotransmission by potentiating 5-HT (serotonin) release, much like amphetamines potentiate, dopamine release. Fenfluramine's serotonin-releasing properties were also initially suspected to mediate the drug's cardiovascular side-effects.

5-HT mediates or regulates a wide variety of behaviors including cognition, emotion, attention, and appetite among others. In addition, there is substantial data indicating that 5-HT is involved in mediating the effects of psychomotor stimulants such as cocaine and 3,4-methylenedioxy-methamphetamine (MDMA or “ecstasy”). There is increasing evidence that serotonergic systems in the brain also regulate dopaminergic reward systems and other factors including the conditioning effects that environmental factors have in maintaining drug taking behavior.

It is now understood that the various physiologic effects of 5-HT are based on its interaction with a number of different 5-HT receptor subtypes which activate different intracellular signaling systems. [Roth, B. L. (ed) The Serotonin Receptors: From Molecular Pharmacology to Human Therapeutics (The Receptors) Humana Press; June 2006; Roth and Shapiro Expert Opin Ther Targets, 5, 685-695, (2001)]. Seven families of 5-HT receptors have been identified including the 5-HT2 receptor family in which 3 sub-types are known (5-HT2A, 5-HT2B and 5-HT2C receptors). The 5-HT2 receptor (5-HT2R) family of serotonin receptors represent key sites of action of serotonin in the brain, and likely comprise the major molecular targets for drugs used in treating a variety of diseases including schizophrenia, depression, anxiety, eating disorders, obsessive-compulsive disorder, chronic pain conditions and obesity.

Drugs may interact with more than one receptor sub-type resulting in potentially undesired side-effects. It is, for example, now clear that, in addition to monoamine transporters, fenfluramine exhibits 5-HT2C receptor agonism in vivo, and that the anorectic functions of fenfluramine are due primarily to this latter activity, at least in laboratory animals (Setola, V., et al. (2005) Mol. Pharmacol. 68, 20-33.) It is also clear that 5-HT2B receptor agonism is likely responsible for the undesirable cardiopulmonary actions of fenfluramine and related drugs (Setola, V. et al. (2003) Mol Pharmacol, 63, 1223-1229; Launay et al. (2002) Nature Medicine October; 8(10):1129-35).

These findings indicate that there is a need in the art for drugs which interact selectively with 5-HT receptor sub-types. In particular, this invention relates to 5-HT2C receptor-selective agonists which preferably exhibit minimal effect on 5-HT2A and 5-HT2B receptors. Selective 5-HT2C receptor agonists can be useful for treatment obesity and related or associated disorders including hypertension, hyperlipidemia, diabetes and cardiovascular disease, and avoid interaction with several related (and unrelated) receptors which have been associated with significant morbidity and mortality, e.g., valvular heart disease associated with activation of the 5-HT2B receptor subtype and hallucinations associated with activation of the 5-HT2A receptor subtype (Egan C T, et al. Psychopharmacology (Berl). 1998 April; 136(4):409-14.)

Selective 5-HT2C receptor agonists can be useful for treatment useful for treatment of depression, anxiety, panic disorder, schizophrenia, OCD, epilepsy, migraine, in addition to obesity. 5-HT2C receptor agonists are further reported (Published PCT application Wo 2006/065600) to be useful for treatment of Alzheimer's Disease and in prevention or treatment of senile plaques as well as in the treatment of sexual dysfunction in males and females, including the treatment of erectile dysfunction.

A number of designed, synthetic compounds have been reported that show 5-HT2C receptor agonistic activity.

Isaac, M. Curr Top Med Chem, 5, 59-67, (2005) is a review that relates to the evidence 5-HT2B/2C receptor agonists, for example, 1-(m-chlorophenyl)-piperazine (mCPP) exhibits activity as an anticonvulsant and 5-HT2C receptor subtype is a target for the treatment of epilepsy. Isaac M., et al. Bioorg. Med. Chem. Lett. (2000) May 10(9):919-921 reports pyrrolo[3,2,1-ij]quinoline derivatives that are 5-HT2C receptor agonists with selectivity over the 5-HT2A receptor which are described as having potential therapeutic applications for treatment of obesity and epilepsy.

Smith et al. Bioorg. Med. Chem. Lett., 15, 1467-1470, (2005) reports synthesis, biological evaluation and structure-activity relationships for a series of 3-benzazepine derivatives as 5-HT(2C) receptor agonists. The compounds were evaluated in [3H] phosphoinositol turnover assays in HEK-293 cells transiently transfected with human 5-HT2C, 5-HT(2A) or 5-HT2B receptors. Compounds were also assessed in a rat feeding model.

Martin et al. (1998) J. Pharmacol. Exp. Therap. “5-HT2C receptor agonists: pharmacological characteristics and therapeutic potential,” 286(2) 913-924 reports selective 5-HT2C receptor agonist activity of:

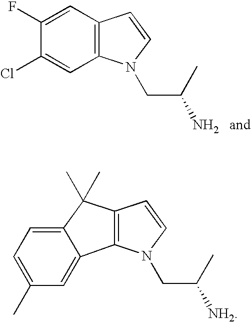

The report focuses on the possible involvement of 5-HT2C agonism in psychiatric disorders employing testing in animal models of such disorders. The results are reported to demonstrate the therapeutic potential of these molecules for OCD and depression.

Bos M, et al. J Med. Chem. 1997 “Novel agonists of 5HT2C receptors. Synthesis and biological evaluation of substituted 2-(indol-1-yl)-1-methylethylamines and 2-(indeno[1,2-b]pyrrol-1-yl)-1-methylethylamines. Improved therapeutics for obsessive compulsive disorder,” August 15; 40(17):2762-9 relates to assessment of 5-HT2C receptor agonists for use in treatment of OCD.

Moreau J L, et al. Eur Neuropsychopharmacol. 1996 “5HT2C receptor agonists exhibit antidepressant-like properties in the anhedonia model of depression in rats,” August; 6(3):169-175 relates to assessment of 5-HT2C receptor agonists in an animal model of depression.

Kimura Y, Hatanaka K, Naitou Y, Maeno K, Shimada I, Koakutsu A, Wanibuchi F, Yamaguchi T. Pharmacological profile of YM348, a novel, potent and orally active 5-HT2C receptor agonist, Eur J. Pharmacol. 2004 January 1; 483(1):37-43 report that YM348, (S)-2-(7-ethyl-1H-furo[2,3-g]indazol-1-yl)-1-methylethylamine is a high affinity 5-HT2C receptor angonist which on oral administration induced penile erections and hypolocomotion in rats.

U.S. Pat. No. 6,962,939 and published US application 2005/197380 relate to indoline derivatives which are selective 5-HT2C receptor agonists and antagonists which are reported to be useful in the treatment of disorders of the central nervous system; damage to the central nervous system; cardiovascular disorders; gastrointestinal disorders; diabetes insipidus, and sleep apnea, and particularly for the treatment of obesity.

U.S. Pat. No. 6,777,407 relates to 5-HT2C selective agonists which are cyclopenta[b][1,4]diazepino[6,7,1-hi]indoles and derivatives thereof and which are reported to be useful for treatment of obsessive-compulsive disorder, panic disorder, depression, anxiety, generalized anxiety disorder, schizophrenia, migraine, sleep disorders, eating disorders, obesity, epilepsy, and spinal cord injury.

U.S. Pat. No. 7,012,089 relates to 5-HT2C receptor agonists or partial agonists which are [1,4]Diazocino[7,8,1-hi]indole derivatives and are reported to be useful as antipsychotic and antiobesity agents.

U.S. Pat. No. 6,953,787 and US published application 2005/020573 relate to 5-HT2C receptor agonists having a basic structure:

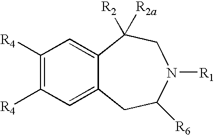

which are reported to be useful in the treatment of obesity and psychiatric disorders.

U.S. Pat. No. 7,071,185, and published US applications US 2006/154920 and 2004/0009970 relate to 5-HT2C receptor agonists or partial agonists which are 1,2,3,4,7,8-hexahydro-6H-[1,4]diazepino[6,7,1-ij]quinoline derivatives and are reported to be useful as antipsychotic and antiobesity agents

Published U.S. application US 2005/026925 relates to 5-HT2C receptor agonists which are piperazine derivatives which are reported to be useful in the treatment and prophylaxis of eating disorders, obesity, and diabetes and generally for treatment of disorders of the central nervous system (depression, anxiety, among others), cardiovascular disorders, gastrointestinal disorders, diabetes insipidus and sleep apnea.

Published U.S. applications 2005/0143452 and 2005/0261347 relate to 5-HTC2 agonists or partial agonists which are dihydrobenzofuranyl alkanamine derivatives and methods for using them to treat a variety of central nervous system disorders such as schizophrenia, psychosis, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, substance abuse, addiction to alcohol and drugs, or a central nervous system deficiency associated with trauma, stroke, or spinal cord injury as well as for treatment of gastrointestinal disorders and obesity.

Published International application WO2000/035922 relates to 2,3,4,4A-tetrahydro-1 h-pyrazino(1,2-a)quinoxalin-5(6 h) one derivates which are 5-HT2C receptor agonists reported to be useful for the treatment of disorders involving the central nervous system such as obsessive-compulsive disorder, depression, anxiety, schizophrenia, migraine, sleep disorders, eating disorders, obesity, type II diabetes, and epilepsy.

Published International application WO2006/065600 relates to n-biaryl and n-arylheteroaryl piperazine derivatives of formula:

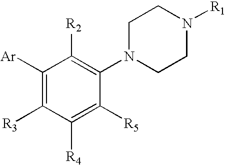

which are reported to be modulators of the 5-HT2C receptor and useful generally for the treatment of 5-HT2C receptor associated diseases or disorders, such as, obesity, Alzheimer's Disease, erectile dysfunction and related disorders.

Published International application WO 2006/077025 relates to morpholines of base formula:

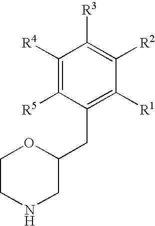

which are reported to be 5-HT2C receptor agonists and useful for the treatment of eating disorders and obesity, various disorders of the central nervous system, cardiovascular disorders, gastrointestinal disorders, diabetes insipidus and sleep apnea.

5-HT2C receptor agonists of this invention are cyclopropane derivatives. Tranylcypromine (trans-2-phenylcyclopropylamine, Parnate®) is a monoamine oxidase inhibitor (MAOI) used as an antidepressant drug. Anorexia is reported to be a side-effect of treatment with tranylcypromine. Kaiser, C. et al. J. Med. Chem. (1962) 5:1243-1264 and Zirkel et al. J. Med. Chem. (1962) 5:1265-1284 have reported the synthesis of a variety of cyclopropyl amine derivatives and the assessment of these derivatives for inhibition of MOA. The compounds, including trans-aminomethyl-2-phenylcyclopropylamine (as the hydrochloride salt), were tested in vivo in rats using the tryptamine potentiation test. trans-Aminomethyl-2-phenylcyclopropylamine was reported not to inhibit MOA. It was more generally reported that MOA inhibition required the presence of a cyclopropane ring and an amino group directly bonded to that ring.

In a related study, Teotino, U. et al. (1967) J. Med. Chem. 10:1091-1096 reported the synthesis of a series of aminomethyl-2-phenylcyclopropane derivatives which were compared with tranylcypromine (2-phenylcyclopropylamine) for possible function as monoamine oxidase (MAO) inhibitors. The compounds reported are amines and certain salts thereof of formula:

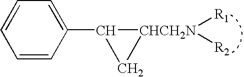

where R₁ is H and R₂ is H, methyl, ethyl, n-propyl, iso-propyl, n-butyl, iso-butyl, sec-butyl, t-butyl, or cyclohexyl and the molecule is in the trans configuration, and where R₁ is H and R₂ is ethyl and the molecule is in the cis configuration, or
where R₁ is methyl and R₂ is methyl or n-butyl; R₁ is ethyl and R₂ is ethyl or benzyl; both R₁ and R₂ are —C₂H₄—OH; both R₁ and R₂ are n-propyl, iso-propyl, n-butyl, iso-butyl or n-pentyl and the molecule is in the trans configuration, and where R₁ and R₂ are both ethyl and the molecule is in the cis configuration; or
where R₁ and R₂ together form a pyrrolidine, piperidine or morpholine ring and the molecule is in the trans configuration. Compounds where R₁ is H, and R₂ is H, methyl, ethyl or cyclohexyl or where R₁ and R₂ together form a pyrrolidine or piperidine ring were reported to have anorexic effect. The reference states that in the series of amines “lower monoalkyl-substituted members exert antidepressant and sympathomimetic effects qualitatively comparable to those of tranylcypromine and amphetamine, without modifying the brain and liver MAO activity.” The dialkylamino compounds are reported to appear to have antiepinephrine and oxytocic activity. None of the compounds studied were associated with any effect on 5-HT receptors.

U.S. published patent application 2002 032199 relates to 5-HT7 receptor antagonist and partial agonists reported to be useful for the treatment of CNS and ocular disorders. In one embodiment, compounds of formula:

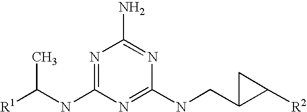

where R² is a phenyl or pyridyl group optionally substituted with one or more of the same or different halogens are disclosed. Certain cycylopropyl amides and amines are disclosed as intermediates useful for the preparation of the above compounds. The intermediates have the structures:

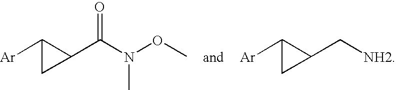

Examples of syntheses are provided in which Ar is a 4-pyridyl group, a 2-fluoropheneyl group or a 4-fluorophenyl group. The synthetic intermediates disclosed are not reported to exhibit any particular therapeutic action.

Published International application WO 2005/007614 relates to monoamine compounds of formula:

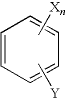

or pharmaceutically acceptable salts thereof,
where Y is

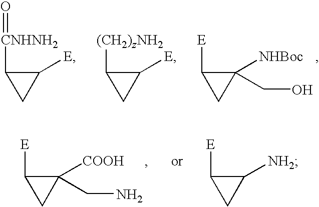

X is independently an electron withdrawing group or an electron donating group; n is an integer from 0 to 3; E is an electron donating group; and z is an integer from 0 to 3, with the proviso that when Y is

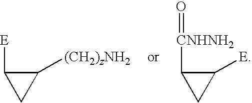

or where the cyclopropyl ring is attached at the carbon substituted with the E, X is not H. The term “electron donating group” is said to include halogens, alkyls, amides, carboxylic acids, amines, hydroxyl, and ether. The term “electron withdrawing group” is said to include carboxyl, ester, carboxamide, cyano, nitro, and trifluoromethyl.

### SUMMARY OF THE INVENTION

This invention relates to compounds which modulate receptors of the 5-HT2 family of receptors, and particularly to compounds which modulate 5-HT2C receptors. In specific embodiments, compounds of this invention selectively modulate the 5-HT2C receptor while exhibiting significantly less or no activity on the 5-HT2B receptor. In specific embodiments, compounds of this invention selectively modulate the 5-HT2C receptor while exhibiting significantly less or no activity on the 5-HT2A receptor. In specific embodiments, the compounds of this invention are agonists for the 5-HT2C receptor. In preferred embodiments, compounds of this invention are selective agonists for the 5-HT2C receptor while exhibiting significantly less or no agonist activity on the 5-HT2A receptor and/or the 5-HT2B receptor. Compounds of this invention are those of Formula I:

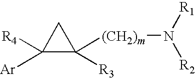

and pharmaceutically acceptable salts, esters and solvates (including hydrates) thereof, where:
m is 1 or 2;
R₁ and R₂ are the same or different and are independently selected from H, or C₁-C₆-alkyl groups;
R₃ and R₄ are the same or different and are independently selected from H, halide (e.g., —F, —Cl, —I or —Br), hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy (—CO—OR), ether, carboxyl (—CO₂—), oxycarbonyl (—O—CO—R′), amino, alkylamino, arylamino, heteroarylamino, amido (—CO—N(R″)₂), aminoacyl (—NR″—CO—R″), imino (—N═C(R)₂), carbamyl (—NR″CO—OR″), urea (—NR′—CO—N(R″)₂ alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate; and
Ar is an aryl group or heteroaryl group which can contain one or more rings at least one of which is aromatic, the aryl and heteroaryl groups are optionally substituted with one or more non-hydrogen substituents, when the aryl or heteroaryl group contains two or more rings, the rings may be linked by a single bond or a linker group or the rings may be fused, the linker group L is selected from alkanediyls (—CH₂—)p where p is 1 to 6 and where one or more —CH₂— moieties can be replaced with a double bond (olefin linkage), a triple bond(alkyne linkage), —O— (ether linkage), —S-(thioether linkage), CO (ketone linkage), NR (amine linkage), —CO—O— (ester linkage), —CO—NR (amide linkage), —NR—CO—O— (carbamyl linkage) or —NR—CO—NR— (a urea linkage) where R is H or an alkyl (e.g., C1-C6 alkyl) or aryl group (e.g., phenyl group).

In specific embodiments, compounds of this invention exhibit no substantial activity as monoamine oxidase inhibitors. In specific embodiments, compounds of this invention exhibit monoamine oxidase inhibition activity substantially lower (10 fold or more lower) than tranylcyproamine.

Compounds of formula I are useful for the treatment of any diseases, conditions, symptoms or disorders associated with a 5-HT2 family receptor and more specifically any such diseases, conditions, symptoms or disorders associated with the 5-HT2C receptor.

In a second embodiment, the invention provides compounds of formula I in which both of R₁ and R₂ are H. The invention provides compounds of formula I in which both of R₁ and R₂ are H and which are 5-HT2C receptor agonists. The invention provides compounds of formula I in which both of R₁ and R₂ are H and which are selective 5-HT2C receptor agonists which exhibit significantly less or no activity for activation of 5-HT2A and/or 5-HT2B receptors.

In a third embodiment, the invention provides compounds of formula I in which both of R₃ and R₄ are H which are 5-HT2C receptor agonists or 5-HT2C selective agonists. In a related embodiment, the invention provides compounds of formula I in which all of R₁₄ are hydrogens and which are 5-HT2C receptor agonists or 5-HT2C selective agonists.

In a fourth embodiment, the invention provides compounds of formula I in which Ar is a phenyl or a substituted phenyl which is substituted with one or more halogens, hydroxyls, alkyl groups, amide groups, ester groups, ether groups, carbamyl groups, amine groups, cyano, isocyano, nitro, haloalkyl (e.g., trifluoromethyl), or hydroxyalkyl which are 5-HT2C receptor agonists or 5-HT2C selective agonists. In a related embodiment, the invention provides compounds of formula I in which Ar is a phenyl or a substituted phenyl and both of R₁ and R₂ and/or both of R₃ and R₄ are hydrogens and which are 5-HT2C receptor agonists or 5-HT2C selective agonists.

In a fifth embodiment, the invention provides compounds of formula I in which Ar is a phenyl substituted with at least an aryl or heteroaryl group which are 5-HT2C receptor agonists or 5-HT2C selective agonists. In related specific embodiments, Ar is biphenyl or in which Ar is a phenyl substituted with a phenyl groups which in turn is substituted with one or more halogens, hydroxyls, alkyl groups, amide groups, ester groups, ether groups, carbamyl groups, amine groups, cyano, isocyano, nitro, haloalkyl (e.g., trifluoromethyl), or hydroxyalkyl which are 5-HT2C receptor agonists or 5-HT2C selective agonists. In another related embodiment, the invention provides compounds of formula I in which Ar is a phenyl substituted with a phenyl or substituted phenyl and both of R₁ and R₂ and/or both of R₃ and R₄ are hydrogens and which are 5-HT2C receptor agonists or 5-HT2C selective agonists.

In a sixth embodiment, the invention provides compounds of formula I wherein Ar is a phenyl which is substituted with at least a furanyl group, particularly with a furan-2-yl group which are 5-HT2C receptor agonists or 5-HT2C selective agonists. In related embodiments, the invention provides compounds of formula I in which Ar is a phenyl substituted with at least a furanyl group and both of R₁ and R₂ and/or both of R₃ and R₄ are hydrogens and which are 5-HT2C receptor agonists or 5-HT2C selective agonists.

In a seventh embodiment, the invention provides compounds of formula I wherein Ar is a phenyl which is substituted with at least a halo phenyl group, particularly with a fluoro-, chloro- or bromo-substituted phenyl group and which are 5-HT2C receptor agonists or 5-HT2C selective agonists. In related embodiments, the invention provides compounds of formula I in which Ar is a phenyl substituted with at one halogen and both of R₁ and R₂ and/or both of R₃ and R₄ are hydrogens and which are 5-HT2C receptor agonists or 5-HT2C selective agonists.

In an eighth embodiment, the invention provides compounds of formula I wherein Ar is a phenyl linked to another aryl or heteroaryl group by a linker group, wherein the linker group is an alkylene linkage, an ether linkage, an amine linkage, a carbamate linkage, an ester linkage or a urea linkage which are 5-HT2C receptor agonists or 5-HT2C selective agonists. In related embodiments, the invention provides compounds of formula I in which Ar is a phenyl linked to an aryl or heteroaryl through a linker group and both of R₁ and R₂ and/or both of R₃ and R₄ are hydrogens and which are 5-HT2C receptor agonists or 5-HT2C selective agonists.

In a ninth embodiment, one or both of R₃ and R₄ are halogens, particularly fluorines. In a related embodiment, one or both of R₃ and R₄ are halogens, particularly fluorines, and R₁ and R₂ are both hydrogens.

In more specific embodiments of formula I and of the first through ninth embodiments listed above, non-hydrogen substituents for aryl or heteroary groups of Ar are selected from halo, azido, cyano, isocyano, alkyl, alkenyl, alkynyl, cycloalkyl, heterocyclyl, aryl, heteroaryl, arylalkyl, heteroarylalkyl, hydroxy, alkoxy, aryloxy, heteroaryloxy, amino, alkylamino, arylamino, acylamino, heteroarylamino, nitro, sulfhydryl, haloalkyl, hydroxylalkyl, imino, amido, phosphonate, phosphinate, ether, thioether, formyl, acyl, carboxyl, carbamyl, acyloxy, silyl, thioether, sulfo, sulfonate, sulfonyl, sulfonamide, and silyl groups.

In additional specific embodiments, non-hydrogen substituents for aryl or heteroaryl groups of Ar are selected from halo, azido, cyano, isocyano, alkyl, alkenyl, alkynyl, haloalkyl, hydroxylalkyl, amido, amino, alkoxyl, benzyl, or amino. In additional specific embodiments, non-hydrogen substituents for aryl or heteroary groups of Ar are selected from one or more of fluoro, chloro, bromo, hydroxy, amido, amino, alkyl, haloalkyl or hydroxyalkyl. In specific embodiments Ar groups herein can have one or two halogen substituents, one or two hydroxyl substituents, one or two alkoxy substitutents (particularly C₁-C₃ alkoxyl substituents) or one or two alkyl substituents (particularly C₁-C₃-alkyl substituents).

In more specific embodiments of formula I and of the first through ninth embodiments listed above, the Ar and —(CH)ₘ—NR₁R₂ groups on the cyclopropane ring are in the trans configuration with respect to each other.

In a specific embodiment of formula I and of the first through ninth embodiments listed above, the compound is mixture of enantiomers in which the (+) enantiomer is present in excess over the (−) enantiomer. Mixtures include those in which the (+) enantiomer represents 75% or more of the mixture, those in which the (=) enantiomer represents 90% or more of the mixture and those in which the (+) enantiomer represents 95% or more of the mixture. In more specific embodiments of formula I and of the first through ninth embodiments listed above, the compound is the (+) enantiomer which is substantially free of the corresponding (−) enantiomer.

An additional aspect of the invention is prodrugs of the compounds of this invention useful for treatment of disorders, conditions, and symptoms as described herein.

In specific embodiments, the compound of Formula I are hydrochloride salts.

Other aspects of the present invention are pharmaceutical compositions comprising a compound of the present invention in combination with a pharmaceutically acceptable carrier wherein the compound is present in the composition in a therapeutically effective amount. In specific embodiments, the invention provides pharmaceutical compositions comprising a therapeutically effective amount of a compound of this invention which is a 5-HT2C receptor agonist or a selective 5-HT2C receptor agonist in combination with a pharmaceutically acceptable carrier.

In a more specific embodiment, the invention provides pharmaceutical compositions for treatment of obesity or the complications thereof comprising a therapeutically effective amount of a compound of this invention which is a 5-HT2C receptor agonist or selective 5-HT2C receptor agonist in combination with a pharmaceutically acceptable carrier. In another specific embodiment, the invention provides pharmaceutical compositions for treatment of eating disorders comprising a therapeutically effective amount of a compound of this invention which is a 5-HT2C receptor agonist or selective 5-HT2C receptor agonist in combination with a pharmaceutically acceptable carrier. In another specific embodiment, the invention provides pharmaceutical compositions for treatment of hypertension, hyperlipidemia, sleep apnea, diabetes, gastrointestinal disorders, and cardiovascular disease comprising a therapeutically effective amount of a compound of this invention which is a 5-HT2C receptor agonist or selective 5-HT2C receptor agonist in combination with a pharmaceutically acceptable carrier.

In another specific embodiment, the invention provides pharmaceutical compositions for treatment of central nervous system disorders or damage to the central nervous system associated with trauma, stroke, or spinal cord injury or complications thereof comprising a therapeutically effective amount of a compound of this invention which is a 5-HT2C receptor agonist or selective 5-HT2C receptor agonist in combination with a pharmaceutically acceptable carrier. In another specific embodiment, the invention provides pharmaceutical compositions for treatment of psychiatric disorders comprising a therapeutically effective amount of a compound of this invention which is a 5-HT2C receptor agonist or selective 5-HT2C receptor agonist in combination with a pharmaceutically acceptable carrier. Other embodiments of the invention are pharmaceutical compositions for treatment of obsessive-compulsive disorder, anxiety, panic disorder, schizophrenia, psychosis, dementia, memory deficit, intellectual deficit associated with Alzheimer's disease, bipolar disorders, adjustment disorders, depression, movement disorders, dystonia, chronic pain, Parkinson's Disease, or Alzheimer's Disease comprising a therapeutically effective amount of a compound of this invention which is a 5-HT2C receptor agonist or a selective 5-HT2C receptor agonist in combination with a pharmaceutically acceptable carrier. Additional embodiments of the invention are pharmaceutical compositions for treatment of epilepsy or migraine comprising a therapeutically effective amount of a compound of this invention which is a 5-HT2C receptor agonist or a selective 5-HT2C receptor agonist in combination with a pharmaceutically acceptable carrier.

Other embodiments of the invention are pharmaceutical compositions for treatment of substance abuse or addiction to alcohol and drugs comprising a therapeutically effective amount of a compound of this invention which is a 5-HT2C receptor agonist or a selective 5-HT2C receptor agonist in combination with a pharmaceutically acceptable carrier Additional embodiments of the invention are pharmaceutical compositions for treatment of sexual dysfunction in males or females and particularly penile dysfunction comprising a therapeutically effective amount of a compound of this invention which is a 5-HT2C receptor agonist or a selective 5-HT2C receptor agonist in combination with a pharmaceutically acceptable carrier.

In another aspect, the invention provides methods of activating a 5-HT2C receptor comprising contacting the receptor with a therapeutically effective amount of a compound of the present invention. In some embodiments, the compound is an agonist of the 5-HT2C receptor or is a selective 5-HT2C receptor agonist. Other aspects of the present invention are methods of treating a 5-HT2C receptor-associated disorder comprising administering to an individual in need of such treatment a therapeutically effective amount of a compound of the present invention or a pharmaceutical composition thereof.

In specific embodiments, the invention relates to methods of treating obesity, eating disorders, gastrointestinal disorders; diabetes, sleep apnea, hypertension, hypertension, hyperlipidemia, and cardiovascular disease comprising administering to an individual in need of such treatment a therapeutically effective amount of a compound of the present invention or a pharmaceutical composition thereof.

In another specific embodiment, the invention provides methods compositions for treatment of central nervous system disorders or damage to the central nervous system associated with trauma, stroke, or spinal cord injury or complications thereof comprising administering to an individual in need of such treatment a therapeutically effective amount of a compound of this invention which is a 5-HT2C receptor agonist or selective 5-HT2C receptor agonist or a pharmaceutical composition thereof.

In another specific embodiment, the invention provides methods compositions for treatment of psychiatric disorders comprising administering to an individual in need of such treatment a therapeutically effective amount of a compound of this invention which is a 5-HT2C receptor agonist or selective 5-HT2C receptor agonist or a pharmaceutical composition thereof. Other embodiments of the invention are methods for treatment of obsessive-compulsive disorder, anxiety, panic disorder, schizophrenia, psychosis, dementia, memory deficit, intellectual deficit associated with Alzheimer's disease, bipolar disorders, adjustment disorders, depression, movement disorders, dystonia, chronic pain, Parkinson's Disease, or Alzheimer's Disease comprising administering to a an individual in need of such treatment a therapeutically effective amount of a compound of this invention which is a 5-HT2C receptor agonist or a selective 5-HT2C receptor agonist or a pharmaceutical composition thereof. Additional methods of the invention are methods for treating epilepsy or migraine by administering to an individual in need of such treatment a compound of the invention which is a 5-HT2C receptor agonist or a selective 5-HT2C receptor agonist or a pharmaceutical composition thereof.

Other embodiments of the invention are methods for treatment of substance abuse or addiction to alcohol and drugs comprising administering to a an individual in need of such treatment a therapeutically effective amount of a compound of this invention which is a 5-HT2C receptor agonist or a selective 5-HT2C receptor agonist or a pharmaceutical composition thereof. Additional embodiments of the invention are methods for treatment of sexual dysfunction in males or females and particularly erectile dysfunction comprising administering to a an individual in need of such treatment a therapeutically effective amount of a compound of this invention which is a 5-HT2C receptor agonist or a selective 5-HT2C receptor agonist or a pharmaceutical composition thereof.

Other aspects of the present invention are methods of decreasing food intake of an individual comprising administering to the individual a therapeutically effective amount of a compound of the present invention or a pharmaceutical composition thereof. In specific embodiments, the compound is a 5-HT2C receptor agonist or selective agonist.

Other aspects to the invention are methods of inducing satiety in an individual comprising administering to the individual a therapeutically effective amount of a compound of the present invention or a pharmaceutical composition thereof. In specific embodiments, the compound is a 5-HT2C receptor agonist or selective agonist.

Additional aspects of the invention are methods of controlling weight gain of an individual comprising administering to the individual suffering from weight control a therapeutically effective amount of a compound of the present invention or a pharmaceutical composition thereof. In specific embodiments, the compound is a 5-HT2C receptor agonist or selective agonist.

The invention also provides medicaments comprising one or more of the compounds of this invention. In specific embodiments, the compound is a 5-HT2C receptor agonist or selective agonist.

The invention further provides methods for producing pharmaceutical compositions comprising admixing one or more compound of the present invention and a pharmaceutically acceptable carrier wherein the composition comprises a therapeutically effective amount or combined amount of the one or more compounds. In specific embodiments, the compound is a 5-HT2C receptor agonist or selective agonist.

Another aspect of the present invention pertains to compounds of the present invention for use in methods of treatment of the human or animal body by therapy. In specific embodiments, the compounds are 5-HT2C receptor agonists or selective agonists.

An additional aspect of the invention is a method for modulating a 2-HT2C receptor in vivo or in vitro which comprises contacting the receptor with one or more compounds of the invention. In specific embodiments, the method is for stimulating or activating the 2-HT2C receptor. In specific embodiments, the compounds are 5-HT2C receptor agonists or selective agonists.

Additional aspects and embodiments of the invention will be apparent in view of the following detailed description and non-limiting examples.

### DETAILED DESCRIPTION OF THE INVENTION

### Definitions

The term “agonist” refers generally to a chemical species that interacts with and activates a receptor, such as one or more of the receptors of the 5-HT2 family of receptors, and initiates a physiological or pharmacological response characteristic of that receptor. The term “antagonist” refers generally to a chemical species that bind to the receptor at the same site as an agonist, but which do not activate the intracellular response initiated by the active form of the receptor, and as such an antagonist can inhibit the intracellular responses by agonists.

As used herein the term “selective 5-HT2C receptor agonist” means an agonist compound that is selective for binding and activation of the 5-HT2C receptor compared to the other receptors of the 5-HT2 family of receptors. An agonist of this invention can be selective for the 5-HT2C receptor over the 5-HT2B receptor, be selective for the 5-HT2C receptor over the 5-HT2A receptor, or be selective for the 5-HT2C receptor over both the 5-HT2B and 5-HT2A receptors. However, in specific embodiments, 5-HT2C receptor agonists of this invention can also exhibit agonist activity with respect to the 5-HT2A receptor. A selective 5HT-2C receptor agonist can exhibit a 5-fold, 10-fold, 20-fold, 30-fold, 40-fold, 50-fold, 60-fold, 70-fold, 80-fold, 90-fold, 100-fold, or 200-fold or more higher activity for the 5-HT2C receptor compared to either or both of the 5-HT2B or 5-HT2A receptors. Selectivity can be assessed for example as illustrated in Table 1, by determining EC₅₀ ratios for different receptors. Any method known in the art to be reliable and accurate for measuring receptor agonist activity can be used to assess selectivity of a given agonist. As understood by one skilled in the art, selectivity can be determined, for example, using a receptor binding assay or a functional assay. In specific embodiments, methods described in the Examples hereof or in methods detailed in references cited herein can be employed. In specific embodiments herein, 5-HT2C receptor agonists of this invention can also exhibit selectively over receptors of 5-HT families other than those of the 5-HT2 family. In specific embodiments herein, 5-HT2C receptor agonists of this invention do not exhibit any substantial antagonist activity for the 5-HT2A or 5-HT2B receptors.

In specific embodiments herein, 5-HT2C receptor agonists may exhibit some level of activity as monoamine oxidase inhibitors. In preferred embodiments, 5-HT2C receptor agonists of this invention do not exhibit any substantial level of activity as monoamine oxidase inhibitors. In a specific embodiment, 5-HT2C receptor agonists of this invention exhibit levels of activity as monoamine oxidase inhibitors that are substantially lower than that exhibited by tranycypromine. In specific embodiments, monoamine oxidase inhibition is measured by a method that is understood in the art to provide reliable and accurate measurement of such inhibition. In particular, methods as described in WO 2005/007614, particularly those methods employing tyramine oxidase, can be employed to assess activity as monoamine oxidase inhibitors. In specific embodiments, 5-HT2C receptor agonists of this invention exhibit activity as monoamine oxidase inhibitors that are 10-fold, or more, lower than that of tranycypromine. In specific embodiments, 5-HT2C receptor agonists of this invention exhibit activity as monoamine oxidase inhibitors that are 25-fold, or more, lower than that of tranycypromine. In specific embodiments, 5-HT2C receptor agonists of this invention exhibit activity as monoamine oxidase inhibitors that is 50-fold or more lower than that of tranycypromine. In specific embodiments, 5-HT2C receptor agonists of this invention exhibt activity as monoamine oxidase inhibitors that is 100-fold or more lower than that of tranycypromine.

In specific embodiments, 5-HT2C receptor agonists exhibit EC₅₀ values for activation of human 5-HT2C receptors of 100 nM or less. In preferred embodiments, 5-HT2C receptor agonists exhibit EC₅₀ values for activation of human 5-HT2C receptors of 25 nM or less. In more preferred embodiments, 5-HT2C receptor agonists exhibit EC₅₀ values for activation of human 5-HT2C receptors of 10 nM or less. In specific embodiments, compounds of this invention exhibit 5-fold or more selectivity as agonists for 5-HT2C receptors compared to 5-HT2B receptors or 5-HT2A receptors as assessed by determination for EC₅₀ ratios as illustrated in Table 1 and the examples herein. In specific embodiments, compounds of this invention exhibit 10-fold or more selectivity as agonists for 5-HT2C receptors compared to 5-HT2B receptors or 5-HT2A receptors as assessed by determination for EC₅₀ ratios as illustrated in Table 1 and the examples herein. In specific embodiments, compounds of this invention exhibit 100-fold or more selectivity as agonists for 5-HT2C receptors compared to 5-HT2B receptors or 5-HT2A receptors as assessed by determination of EC₅₀ ratios as illustrated in Table 1 and the examples herein.

The term “alkyl” refers to a monoradical of a branched or unbranched (straight-chain or linear) saturated hydrocarbon and to cycloalkyl groups having one or more rings. Unless otherwise indicated preferred alkyl groups have 1 to 30 carbon atoms and more preferred are those that contain 1-22 carbon atoms. Short alkyl groups are those having 1 to 6 carbon atoms including methyl, ethyl, propyl, butyl, pentyl and hexyl groups, including all isomers thereof. Long alkyl groups are those having 8-30 carbon atoms and preferably those having 12-22 carbon atoms as well as those having 12-20 and those having 16-18 carbon atoms. The term “cycloalkyl” refers to cyclic alkyl groups having preferably 3 to 30 carbon atoms having a single cyclic ring or multiple condensed rings. Cycloalkyl groups include, by way of example, single ring structures such as cyclopropyl, cyclobutyl, cyclopentyl, cyclohexyl, cyclooctyl, and the like, or multiple ring structures such as adamantanyl, and the like. Unless otherwise indicated alkyl groups, including cycloalkyl, groups are optionally substituted as defined below. (The term “radical” is used in a formal sense herein in definition of chemical terms. The use of the term radical is not intended to indicate that the compounds of the invention use or contain free radicals per se.)

The term “alkenyl” refers to a monoradical of a branched or unbranched unsaturated hydrocarbon group having one or more double bonds and to cycloalkenyl group having one or more rings wherein at least one ring contains a double bond. Unless otherwise indicated preferred alkyl groups have 1 to 30 carbon atoms and more preferred are those that contain 1-22 carbon atoms. Alkenyl groups may contain one or more double bonds (C═C) which may be conjugated or unconjugated. Preferred alkenyl groups are those having 1 or 2 double bonds and include omega-alkenyl groups. Short alkenyl groups are those having 2 to 6 carbon atoms including ethylene (vinyl), propylene, butylene, pentylene and hexylene groups including all isomers thereof. Long alkenyl groups are those having 8-30 carbon atoms and preferably those having 12-22 carbon atoms as well as those having 12-20 carbon atoms and those having 16-18 carbon atoms. The term “cycloalkenyl” refers to cyclic alkenyl groups of from 3 to 30 carbon atoms having a single cyclic ring or multiple condensed rings in which at least one ring contains a double bond (C═C). Cycloalkenyl groups include, by way of example, single ring structures (monocyclic) such as cyclopropenyl, cyclobutenyl, cyclopentenyl, cyclohexenyl, cyclooctenyl, cylcooctadienyl and cyclooctatrienyl as well as multiple ring structures. Unless otherwise indicated alkenyl groups including cycloalkenyl groups are optionally substituted as defined below.

The term “alkynyl” refers to a monoradical of an unsaturated hydrocarbon having one or more triple bonds (C—C). Unless otherwise indicated preferred alkyl groups have 1 to 30 carbon atoms and more preferred are those that contain 1-22 carbon atoms. Alkynyl groups include ethynyl, propargyl, and the like. Short alkynyl groups are those having 2 to 6 carbon atoms, including all isomers thereof. Long alkynyl groups are those having 8-22 carbon atoms and preferably those having 12-22 carbon atoms as well as those having 12-20 carbon atoms and those having 16-18 carbon atoms. The term “cycloalkynyl” refers to cyclic alkynyl groups of from 3 to 30 carbon atoms having a single cyclic ring or multiple condensed rings in which at least one ring contains a triple bond (C—C). Unless otherwise indicated alkyl groups including cycloalkyl groups are optionally substituted as defined below.

The term “alicyclyl” generically refers to a monoradical that contains a carbon ring which may be a saturated ring (e.g., cyclohexyl) or unsaturated (e.g., cyclohexenyl) but is not aromatic (e.g., the term does not refer to aryl groups). Ring structures have three or more carbon atoms and typically have 3-10 carbon atoms. As indicated above for cycloalkane, cycloalkenes and cycloakynes, alicyclic radical can contain one ring or multiple rings (bicyclic, tricyclic etc.)

The term “heterocyclyl” generically refers to a monoradical that contains at least one ring of atoms, which may be a saturated, unsaturated or aromatic ring wherein one or more carbons of the ring are replaced with heteroatoms (a non-carbon atom) To satisfy valence the heteroatom may be bonded to H or a substituent groups. A ring may contain one or more different heteroatoms. Ring carbons may be replaced with —O—, —S—, —NR—, —N═, —PR—, or —POR among others, where R is an alkyl, aryl, heterocyclyl or heteroaryl group. Preferred heteroatoms at —O—, —S—, —NR— and —N═. Heterocyclyl groups include those containing 3 to 30 carbon atoms and those carrying 1-6 heteroatoms which may be the same or different.

The term “aryl” refers to a monoradical containing at least one aromatic ring. The radical is formally derived by removing a H from a ring carbon. Aryl groups contain one or more rings at least one of which is aromatic. Rings of aryl groups may be linked by a single bond or a linker group or may be fused. Exemplary aryl groups include phenyl, biphenyl and naphthyl groups. Aryl groups include those having from 6 to 30 carbon atoms and those containing 6-12 carbon atoms. Unless otherwise noted aryl groups are optionally substituted as described herein.

The term “arylalkyl” refers to a group that contains at least one alkyl group and at least one aryl group, the aryl group may be substituted on the alkyl group (e.g., benzyl, —CH₂—C₆H₅) or the alkyl group may be substituted on the aryl group (e.g., tolyl, —C₆—H₄—CH₃). Unless otherwise noted either the alkyl or the aryl portion of the arylalkyl group can be substituted as described herein.

The term “heteroaryl” refers to a group that contains at least one aromatic ring in which one or more of the ring carbons is replaced with a heteroatom (non-carbon atom). To satisfy valence the heteroatom may be bonded to H or a substituent groups. Ring carbons may be replaced with —O—, —S—, —NR—, —N═, —PR—, or —POR among others, where R is an alkyl, aryl, heterocyclyl or heteroaryl group. Heteroaryl groups may include one or more aryl groups (carbon aromatic rings) heteroaromatic and aryl rings of the heteroaryl group may be linked by a single bond or a linker group or may be fused. Heteroaryl groups include those having aromatic rings with 5 or 6 ring atoms of which 1-3 ring atoms are heteroatoms. Preferred heteroatoms are —O—, —S—, —NR— and —N═. Heteroaryl groups include those containing 6-12 carbon atoms. Unless otherwise noted heteroaryl groups are optionally substituted as described herein.

The term “heteroarylalkyl” is analogous to the term “arylalkyl” above. It refers to a group that contains at least one alkyl group and at least one heteroaryl group, the heteroaryl group may be substituted on the alkyl group or on the heteroaryl group. A heteroaryl group may also contain one or more aryl groups (one or more carbon aromatic rings). Unless otherwise noted either the alkyl or the aryl portion of the arylalkyl group can be substituted as described herein.

The term “alkenylene” is used herein to refer to a bivalent radical derived from an alkyl group, which functions as a linker between two other chemical groups, e.g., between two allicyclic, heterocyclic, aryl, or heteroaryl rings. The term alkanediyl can also be used. Bivalent radical linker groups in the compounds of this invention include alkenylene groups and bivalent radicals in which one or more —CH₂— of an alkenylene group are replaced with an —O—, —S—, —CO, —NR—CO—, —O—CO—, or —NR—CO—NR— group where each R, independent of other R, is H, alkyl or aryl group. Carbon atoms of the alkenylene and other bivalent linker groups are optionally substituted as described herein. In particular embodiments, the bivalent linker groups are substituted with one or more —OH or —NH₂ groups.

In compounds herein containing two or more rings (aryl, heteroaryl, alicyclyl, or heterocyclyl), rings can be directly linked to each other through a single bond or through a bivalent radical linker as described above.

The term “oxy” refers to —O— and is used in combination with descriptors for other organic radical to indicate —O-M groups where M is alkyl, alkenyl, alkynyl, aryl, arylalkyl, heterocyclyl, heteroaryl or heteroarylalkyl, as in alkoxy, alkenoxy, alkynoxy, aryloxy, arylalkoxy, heteroaryloxy, heterocyclyloxy.

The term “alkoxy” (or alkoxide) refers to a —O-alkyl group, where alkyl groups are as defined above. The term alkenoxy (alkenoxide) refers to a —O-alkenyl group where alkenyl groups are as defined above and wherein a double bond is preferably not positioned at the carbon bonded to the oxygen. The term alkynoxy (alkynoxide) refers to a —O-alkynyl group where alkynyl groups are as defined above and wherein a triple bond is not positioned at the carbon bonded to the oxygen. Unless otherwise noted, alkyl, alkenyl and alkynyl portions of the alkoxy, alkenoxy and alkynoxy groups are optionally substituted as described herein.

The terms “hydroxyl” or “hydroxide” refer to —OH.

The terms “aryloxy,” “heteroaryloxy” and “heterocyclyloxy” refer to the —O-M group where M is an aryl, heteroaryl or heterocyclyl radical, respectively.

The term “acyl” refers to the radical —CO—R′ where R′ is an alkyl, alkenyl, alkynyl, aryl, heterocyclyl, or heteroaryl radical as described above and CO is C═O (a carbonyl group). The term “formyl” refers to the —COH group. The term “acetyl” refers to —CO—CH₃.

The term “acyloxy” refers to the radical —CO—O—R′ where R′ is an alkyl, alkenyl, alkynyl, aryl, heterocyclyl, or heteroaryl radical as described above.

The term “carboxy” refers to the group —CO—OH or its anionic form —COO— (carboxylate.)

The term “oxycarbonyl” refers to the radical —O—CO—R′ where R′ is an alkyl, alkenyl, alkynyl, aryl, heterocyclyl, or heteroaryl radical as described above.

The term “ether group” (also alkoxyalkyl) is used herein to refer to an alkyl group in which one or more —CH₂— groups are replaced with —O—. Unless otherwise stated preferred alkoxyalkyl groups have from 3 to 30 carbon atoms and more preferably have 6 to 22 carbon atoms. Ether groups include groups of the formula: —[(CH₂)a-O-]b—CH₃ where a is 1-10 and b is 1-6. More specifically, a can be 2, 3 or 4 and b can be 1, 2 or 3.

The term “thioether group” or “thioalkoxyalkyl” refers to an alkyl group in which one or more —CH₂— groups are replaced with —S—. Unless otherwise specified, preferred thioalkoxyalkyl groups have from 3 to 30 carbon atoms and more preferably have 6 to 22 carbon atoms. Thioalkoxylalkyl groups include groups of the formula: —[(CH₂)a-S-]b-CH₃ where a is 1-10 and b is 1-6. Alkoxyalkyl and thioalkoxyalkyl groups can be branched by substitution of one or more carbons of the group with alkyl groups.

The term “sulfenyl” refers to the radical —S—R′ where R′ is an alkyl, alkenyl, alkynyl, aryl, heterocyclyl, or heteroaryl radical as described above. The term “sulfhydryl” refers to the —SH group.

The term “sulfonyl” refers to the radical —SO₂—R′ where R′ is an alkyl, alkenyl, alkynyl, aryl, heterocyclyl, or heteroaryl radical as described above.

The term “sulfonate” refers to the radical —SO₃—R″ where R″ is hydrogen, alkyl, alkenyl, alkynyl, aryl, heterocyclyl, or heteroaryl radical as described above.

An “alkyl sulfonate” group refers to a sulfonate group wherein R″ is alkyl. An “aryl sulfonate” group refers to an sulfonate group wherein at least one R″ is aryl. The group —SO₃H can be in the ionic form —SO₃—.

The term “amino” refers generically to a —N(R″)₂ group wherein R″ independently of other R″ is hydrogen, alkyl, alkenyl, alkynyl, aryl, heterocyclyl, or heteroaryl radical as described above. Two of R″ may be linked to form a ring. An “alkyl amino” group refers to an amino group wherein at least one R″ is alkyl. An “aryl amino” group refers to an amino group wherein at least one R″ is aryl.

The term “amido” refers generically to an —CO—N(R″)₂ group wherein R′ independently of other R′ is hydrogen, alkyl, alkenyl, alkynyl, aryl, heterocyclyl, or heteroaryl radical as described above. Two of R″ may be linked to form a ring. An “alkyl amido” group refers to an amido group wherein at least one R″ is alkyl. An “aryl amido” group refers to an amido group wherein at least one R″ is aryl.

The term “aminoacyl” group “refers generically to an —NR″—CO—R″ group wherein R″ independently of other R″ is hydrogen, alkyl, alkenyl, alkynyl, aryl, heterocyclyl, or heteroaryl radical as described above. Two of R″ may be linked to form a ring. An “alkyl aminoacyl” group refers to an aminoacyl group wherein at least one R″ is alkyl. An “aryl amido” group refers to an aminoacyl group wherein at least one R″ is aryl.

The term “carbamyl” refer to an —NR″—CO—OR″ group wherein R″ independently of other R″ is hydrogen, alkyl, alkenyl, alkynyl, aryl, heterocyclyl, or heteroaryl radical as described above. The term includes the —NHCO—OH (also —NHCO—O—) group and the —NHCO—OR″ group.

The term “imine” refer generically to an —N═CR₁₂ group or an —CR″═NR″ group wherein R″ independently of other R″ is hydrogen, alkyl, alkenyl, alkynyl, aryl, heterocyclyl, or heteroaryl radical as described above. Two of R″ may be linked to form a ring. An “alkyl imine” group refers to an imine group wherein at least one R″ is alkyl. An “aryl imine” group refers to an imine group wherein at least one R″ is aryl.

The term “urea’ refers herein to a urea group —NR″—CO—N(R″)₂ wherein R″ independently of other R″ is hydrogen, alkyl, alkenyl, alkynyl, aryl, heterocyclyl, or heteroaryl radical as described above. In specific embodiments, all of R″ are H. In other embodiments, the urea has the structure —NH—CO—NH—R′ wherein R′ is an alkyl, alkenyl, alkynyl, aryl, heterocyclyl, or heteroaryl radical as described above.

The term “phosphonate” refers to either a —PO(OR″)₂ group or an —O—PO(OR″) group where R″ independently of other R″ is hydrogen, alkyl, alkenyl, alkynyl, aryl, heterocyclyl, or heteroaryl radical as described above. Two of R″ may be linked to form a ring. An “alkyl phosphonate” group refers to a phosphonate group wherein at least one R″ is alkyl. An “aryl phosphonate” group refers to a phosphonate group wherein at least one R″ is aryl.

The term “phosphinate” refer to either a —PO(OR′) group or a —OPOR″ group where R″ independently of other R″ is hydrogen, alkyl, alkenyl, alkynyl, aryl, heterocyclyl, or heteroaryl radical as described above. Two of R″ may be linked to form a ring. An “alkyl phosphinate” group refers to a phosphinate group wherein at least one R″ is alkyl. An “aryl phosphinate” group refers to a phosphinate group wherein at least one R″ is aryl.

Silyl refer generally to the group —S(R)₃ where each R independently of other R is hydrogen, an optionally substituted hydrocarbyl group, including optionally substituted alkyl, alkenyl, alkynyl, aryl, or arylalkyl groups, an optionally substituted heterocyclyl group, heteroaryl group or heteroarylalkyl group. In specific embodiments, R is hydrogen, alkyl groups, particularly alkyl groups having 1-10 carbon atoms (including those having 1-6 carbon atoms) or aryl groups, particularly phenyl groups or biphenyl groups. R is most generally optionally substituted as described herein, but in specific embodiments, R can be substituted with one or more halogens (particularly fluorines).

The term “haloalkyl” refers to an alkyl as defined herein substituted by one or more halides (e.g., F—, Cl—, I—, Br—) as defined herein, which may be the same or different. A haloalkyl group may, for example, contain 1-10 halide substituents. Representative haloalkyl groups include, by way of example, trifluoromethyl, 3-fluorododecyl, 12,12,12-trifluorododecyl, 2-bromooctyl, 3-bromo-6-chloroheptyl, and the like. Haloalkyl groups include fluoroalkyl groups. A perhaloalkyl group refers to an alkyl group in which all H have been replaced with halogen atoms. A perfluoroalkyl group is an alkyl group in which all H have been replaced with fluorine. Exemplary perhaloalkyl groups include trifluoromethyl and pentafluoroethyl groups.

The term “hydroxylalkyl” refer to an alkyl group substituted by one or more hydroxyl groups. A hydroxyalkyl group may, for example, contain 1-10 hydroxy substituents. An exemplary hydroxyalkyl group is hydroxymethyl (—CH₂—OH).

Alkyl, alkenyl, alkynyl, aryl, heterocyclyl and heterocyclyl groups may be substituted or unsubstituted. These groups may be optionally substituted as described herein and may contain non-hydrogen substituents dependent upon the number of carbon or other atoms in the group and the degree of unsaturation of the group. Unless otherwise indicated substituted alkyl, alkenyl alkynyl aryl, heterocyclyl and heterocyclyl groups preferably contain 1-10, and more preferably 1-6, and more preferably 1, 2 or 3 non-hydrogen substituents.

Optional substitution refers to substitution with one or more of the following functional groups:

nitro, azido, cyano, isocyano, halogen (Cl, F, Br or I), hydroxyl, alkyl (including C₁-C₆ alkyl or C₁-C₃ alkyl), alkenyl, alkynyl, aryl, heteroaryl, arylalkyl, heteroarylalkyl, heterocyclyl, acyl, formyl, acetyl, haloalkyl, haloaryl, alkyloxy (including C₁-C₆ alkoxy or C₁-C₃ alkoxy), alkenoxy, alkynoxy, aryloxy, benzyloxy, phenyloxy (benzoyl), acyloxy, alkyl acyloxy, oxycarbonyl, alkyl oxycarbonyl —NH₂ (or —NH₃+), amino, alkylamino, arylamino, amido, alkyl amido, arylamido, —CO—NH₂, imino, alkyl imino, aryl imino, ether, thioether, —SH, sulfenyl, alkyl sulfenyl (including C₁-C₆ alkyl sulfenyl and C₁-C₃ alkyl sulfenyl), hydroxyalkyl, haloalkyl, fluoroalkyl, perfluoroalkyl, trifluoromethyl, sulfonate, sulfonyl, phosphonate, phosphinate or silyl, wherein alkyl, alkenyl, alkynyl, aryl, heteroaryl, arylalkyl, heteroarylalkyl, and heterocyclyl groups of the substituents are in turn optionally substituted with one or more of: nitro, azido, cyano, isocyano, halogen, —NH₂ (or —NH₃⁺), hydroxyl, —CO—NH₂, —COOH (or carboxylate) or —SH.

Exemplary aryl and heteroaryl groups are illustrated in Scheme X.

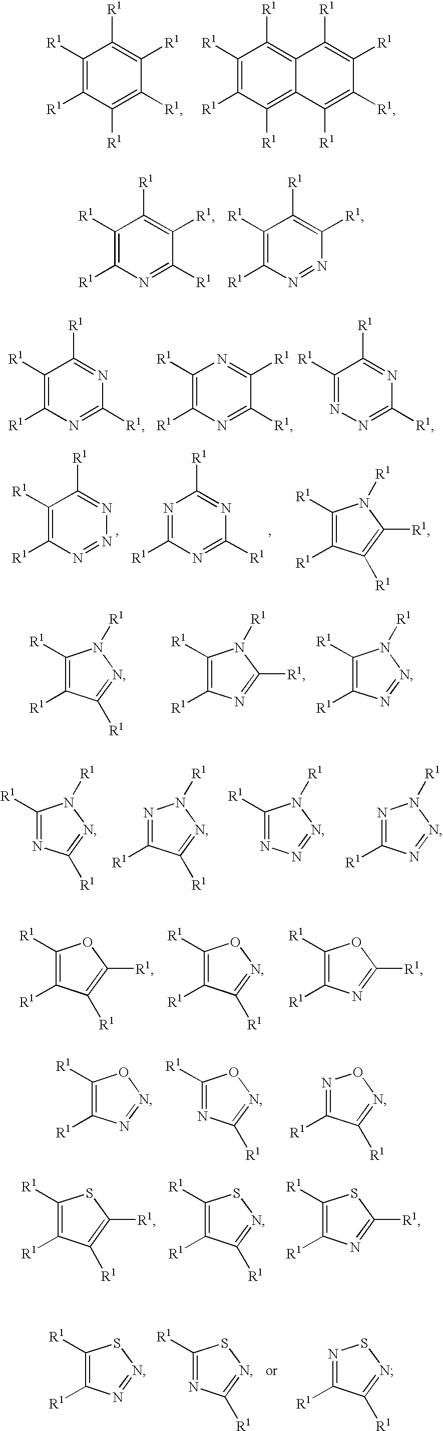

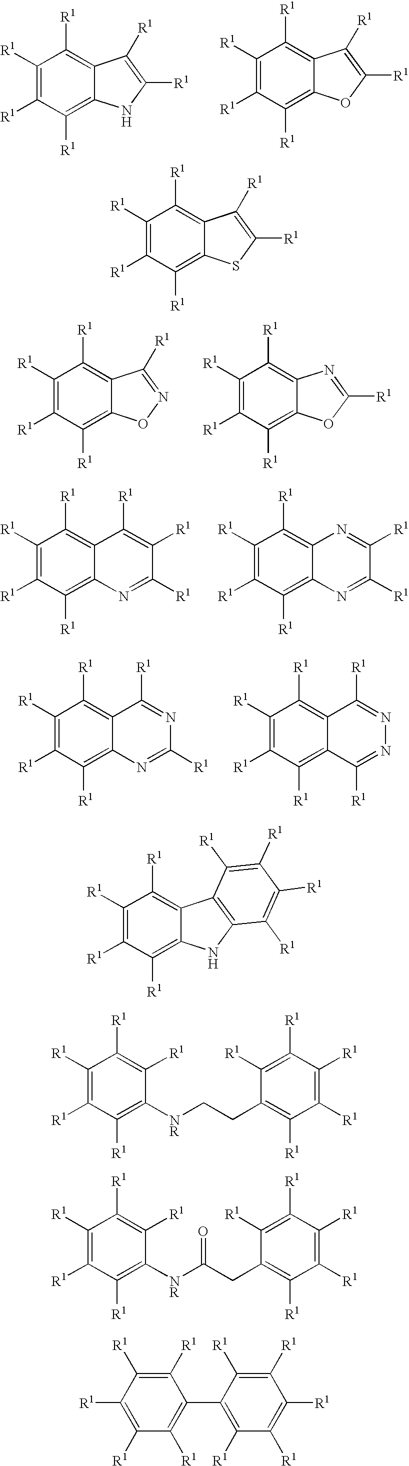

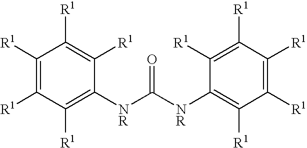

where each R¹, independent of other R¹, is hydrogen or a non-hydrogen substituent and one of R¹ is the cyclopropyl ring of formula I.

Compounds of this invention are those of Formula I:

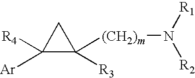

and pharmaceutically acceptable salts, esters and solvates (including hydrates) thereof, where:
m is 1 or 2;
R₁ and R₂ are the same or different and are independently selected from H, or C1-C6-alkyl groups;
R₃ and R₄ are the same or different and are independently selected from H, halide (e.g., —F, —Cl, —I or —Br), hydroxy, sulfhydral (—SH), nitro (—NO₂), azido (—N3), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy (—CO—OR), ether, carboxyl (—CO₂—), oxycarbonyl (—O—CO—R′), amino, alkylamino, arylamino, heteroarylamino, amido (—CO—N(R″)₂), aminoacyl (—NR″˜CO—R″), imino (—N═C(R)₂), carbamyl (—NR″CO—OR″), urea (—NR′—CO—N(R″)₂ alkyl sulfide, alkenyl sulfide, alkynyl sulfide, aryl sulfide, heteroaryl sulfide, thioether, sulfonate, sulfonyl, sulfinyl, sulfonamido,
silyl, phosphonate, or phosphinate; and
Ar is an aryl group or heteroaryl group which can contain one or more rings at least one of which is aromatic, the aryl and heteroaryl groups are optionally substituted with one or more non-hydrogen substituents, when the aryl or heteroaryl group contains two or more rings, the rings may be linked by a single bond or a linker group or the rings may be fused, the linker group L is selected from alkanediyls (—CH2-)p where p is 1 to 6 and where one or more —CH₂— moieties can be replaced with a double bond (olefin linkage), a triple bond(alkyne linkage), —O— (ether linkage), —S-(thioether linkage), CO (ketone linkage), NR (amine linkage), —CO—O— (ester linkage), —CO—NR (amide linkage), —NR—CO—O— (carbamyl linkage) or —NR—CO—NR— (a urea linkage) where R is H or an alkyl (e.g., C1-C6 alkyl) or aryl group (e.g., phenyl group).

In a specific embodiment, Ar is a group other than a 4-pyridyl, 2-fluorophenyl or 4-fluorophenyl group.

In specific embodiments, Ar is a substituted phenyl group. In specific embodiments, when n is 1 and R₃ is F, Ar is a group other than a para-substituted phenyl group. In specific embodiments, when n is 1 and R₃ is F, Ar is a group other than a para-substituted phenyl group in which the substituent is an electron donating group. In specific embodiments, when n is 1 and R₃ is F, Ar is a group other than a para-substituted phenyl group in which the substituent is an electron donating group. In specific embodiments, when n is 1 and R₃ is F, Ar is a group other than a para-substituted phenyl group in which the substituent is an electron withdrawing group. In specific embodiments, when n is 1 and R₃ is F, Ar is a group other than a para-fluoro-substituted phenyl group. In specific embodiments, when n is 1 and R₃ is F, Ar is a group other than a para-substituted phenyl group where the substituent is a halogen, an alkyl, an amide, a carboxylic acid, an amines, a hydroxyl, or an ether. In specific embodiments, when n is 1 and R₃ is F, Ar is a group other than a para-substituted phenyl group where the substituent is a carboxyl, an ester, a carboxamide, a cyano, a nitro, or a trifluoromethyl.

In other specific embodiments, when n is 1 and R₃ is F, Ar is a biphenyl or substituted biphenyl group. In specific embodiments, when n is I and R₃ is F, Ar comprises a heteroaryl group. In specific embodiments, when n is 1 and R₃ is F, Ar comprises a heteroaryl group linked to an aryl group or another heteroaryl group.

In specific embodiments, when n is 2, R₃ is a group other than an electron donating group. In specific embodiments, when n is 2 and Ar is a phenyl or substituted phenyl, R₃ is a group other than a halogen. In specific embodiments, when n is 2 and Ar is a phenyl or substituted phenyl, R₃ is a group other than a fluorine.

In other specific embodiments, when n is 2 and R₃ is F, Ar is a biphenyl or substituted biphenyl group. In specific embodiments, when n is 2 and R₃ is F, Ar comprises a heteroaryl group. In specific embodiments, when n is 2 and R₃ is F, Ar comprises a heteroaryl group linked to an aryl group or another heteroaryl group.

In specific embodiments R₄ is a group other than COOH. In specific embodiments, R₃ is a group other than F.

In other embodiments, Ar is one of the heteroaryl groups of Scheme X. In other embodiments, Ar is a heteroaryl group in which a ring C or N is optionally substituted as defined herein.

In specific embodiments, the compounds of the invention have formula II:

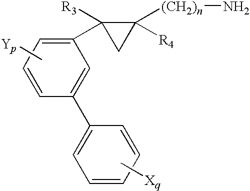

or formula III:

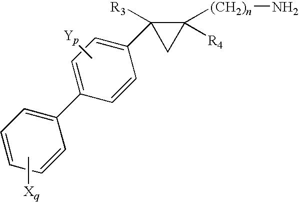

where n, R₃ and R₄ are as defined for formula I and p and q zero or integers 1-4, preferably 1, 2 or 3 and X and Y are optional substitutents as defined herein. In preferred embodiments, Y and X, independently of each other and of other X and Y, are halogens, hydroxyls, alkyl, haloalkyl (e.g., trifluoromethyl), cyano, isocyano, nitro, amine, amide, carboxylic acid, or ester. In preferred embodiments, p is 0 and q is 1. In other embodiments, p is 0, q is 1 and X is a para-substituent. More preferred X and y are halogen, fluoride, hydroxyl, trifluormethyl, cyano and amide. In specific embodiments Ar is a phenyl group linked to a heteroaryl group. In specific embodiments the heteroaryl group is a furyl group. In specific embodiments of formulas II and III, n is 1. In other specific embodiments, R₃ and R₄ are independently H or halogen.

In a specific embodiment, the compounds of the invention have the formula IV:

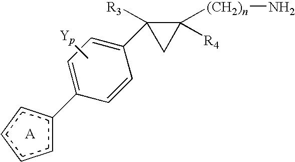

where n, R₃, R₄, p and Y are as defined for formulas I or II and III and the A ring is a heteroaryl ring. In specific embodiments, the A ring is a five-member ring, in other embodiments the A ring is a six-member ring. In another embodiment the A ring is optionally substituted on a ring carbon or a ring nitrogen. In specific embodiments the A ring is a furanyl ring. In other embodiment, the A ring is a pyrrole ring or a thiophene ring. In yet other embodiments, the A ring is one of the heteroaryl rings of Scheme X. In specific embodiments, p is zero or p is 1, 2 or 3. In specific embodiments, Y, dependently of other Y, stet halogens, hydroxyls, alkyl, haloalkyl (e.g., trifluoromethyl), cyano, isocyano, nitro, amine, amide, carboxylic acid, or ester groups In preferred embodiments, p is 0.

In specific embodiments of formulas II, III, and IV n is 1. In other specific embodiments of these formulas, R₃ and R₄ are independently H or halogen. In other specific embodiments of these formulas, R₃ and R₄ are both hydrogens.

In specific embodiments of formulas II, III and IV, the compound is a trans isomer with respect to the alkyl amine group and the aryl or heteroaryl group on the cyclopropane ring. In specific embodiments of Formulas II, III and IV, the compound is a (+) enantiomer

### Synthetic Methods

Compounds of this invention are prepared employing methods as described herein or are prepared by routine modification or adaptation of the methods herein, for example, by selection of starting materials, or variation of reagents, solvents and/or purification methods, in view of what is known in the art.

trans- and cis-1-Aminomethyl-2-phenylcyclopropane are prepared starting from styrene (Scheme 1). Cyclopropanation was carried out using ethyl diazoacetate in the presence of Cu(acac)₂ as a catalyst (Yoshida, S. et al. J. Med. Chem. 2004 47:1796-1806). In this reaction, the product ester is obtained as a 2:1 racemic mixture of the trans and cis isomers. After separation of the stereoisomers on silica gel, the resulting esters trans-3a and cis-3b isomer are converted to the corresponding amines 6a and 6b, respectively, employing a standard sequence of reactions involving amide formation, followed by borane reduction.

N-monomethylation of the amino group in tranylcypromine (n=0) and of the elongated cyclopropylmethylamine derivative (n=1) is carried out by reduction of the corresponding N-formyl derivatives. Formylation of the amine with acetic formic anhydride followed by in situ borane-dimethyl sulfide reduction affords the corresponding N-methylamines (Krishnamurthy, S. (1982) Tetrahedron Lett., 23, 3315-3318). The products obtained in this way are free of any contamination by the product of dialkylation. The N,N-dimethylamine derivative of tranylcypromine is prepared by reductive N-alkylation using an excess of 37% aqueous formaldehyde and NaBH₃CN according to a literature procedure (Scheme 2) (Balboni, G et al. (2003) Bioorg Med Chem, 11, 5435-5441; Arvidsson et al. (1988) J. Med. Chem., 31, 92-99.) The mono-isopropyl and mono-benzylamine analogs are prepared by similar reductive N-alkylation protocols (Kawamoto, H., et al. (1999) J. Med. Chem., 42, 5061-5063; Chandrasekhar, S., et al. (2000) Synlett, 1655-1657.)

Substituted phenyl and naphthyl derivatives are synthesized from the corresponding halogenated styrenes and 2-vinyinaphthalene employing the same method (Scheme 3) as used in the preparation of the 1-aminomethyl-2-phenylcyclopropanes 6a and 6b.

Substituted phenyl and substituted biphenyl compounds 26-30 are synthesized (Scheme 4) using the bromophenyl derivative 13 by the Suzuki coupling (Miyaura, N.; Suzuki, A. Chem. Rev. 1995 95:245.)

Pure enantiomers of trans-1-amino-methyl-2-phenylcyclopropane are prepared as illustrated in Scheme 5. The intermediate carboxylic acid formed in the cyclopropanation step is initially converted to its diastereomeric amides. This is done by coupling the racemic carboxylic acid with (R)-phenylglycinol. (Arvidsson, et al. (1988) J. Med. Chem., 31, 92-99). The resulting diastereomeric pair is then separated by use of silica gel column chromatography. The choice of (R)-phenylglycinol as the alcohol component is based on its successful use in the resolution of other racemic carboxylic acids and the ease of cleavage of the resulting diastereomers under acidic conditions (N,O-acyl transfer occurs). The carboxylic acids are then converted individually to (+)- and (−)-trans-1-aminomethyl-2-phenylcyclopropanes using the same method described above (Zhang, X., et al. (2000) J. Med. Chem., 43, 3923-3932, (2000); Overberger et al. (1971) Macromolecules, 9, 718-722.) In general, compounds of this invention can be prepared in optically pure form using classical chemical resolution methods by fractional crystallization of acid salts or by conversion to appropriate amides using optically pure amines as is illustrated in Scheme 5.

Compounds in which Ar is a substituted phenyl are prepared by the method of Scheme 2 employing an appropriate styrene derivative, commercially available or synthesized from the appropriate benzaldehyde derivative by use of the Wittig chemistry. This method is particularly useful for preparation of dihalogenated phenyl derivatives.

Another exemplary method for preparation of monosubstituted phenyl derivatives is illustrated in Scheme 6. The N-Boc protected aminomethylcyclopropane derivatives illustrated in Scheme 6 are very useful intermediates for the preparation of other analogues, for example, esters, amides, ethers, carbamates, and substituted amines. The bromo group can be converted to a carboxyl or amine. In turn the acid so formed can be converted to ester and amide derivatives, or reduced to alcohol to allow subsequent ether formation. Amines can also be converted to amides and urethanes by reaction with acid chlorides and aryl- and alkylisocyanates.

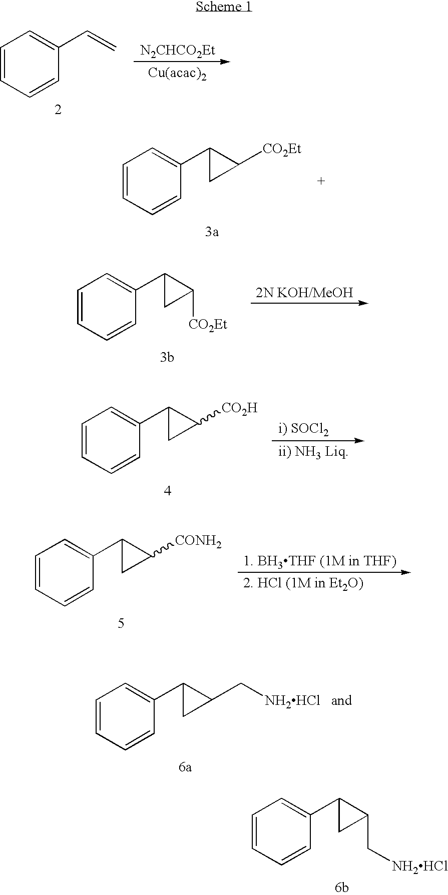

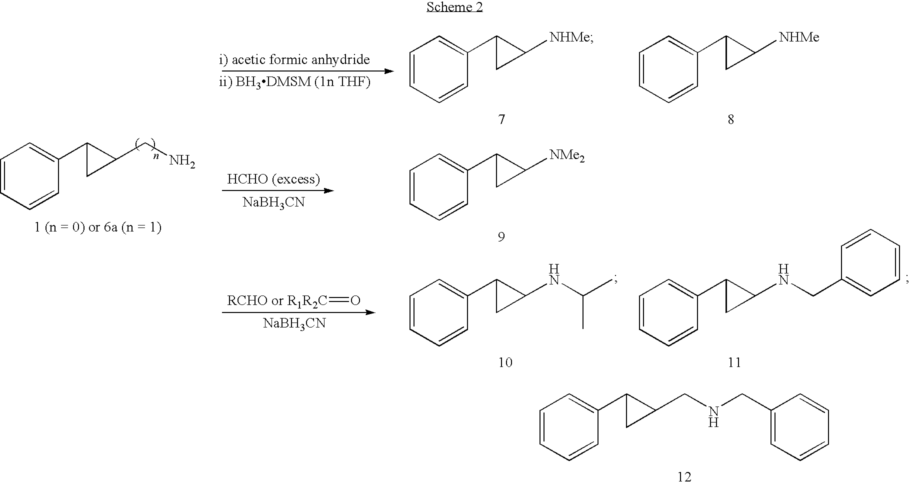

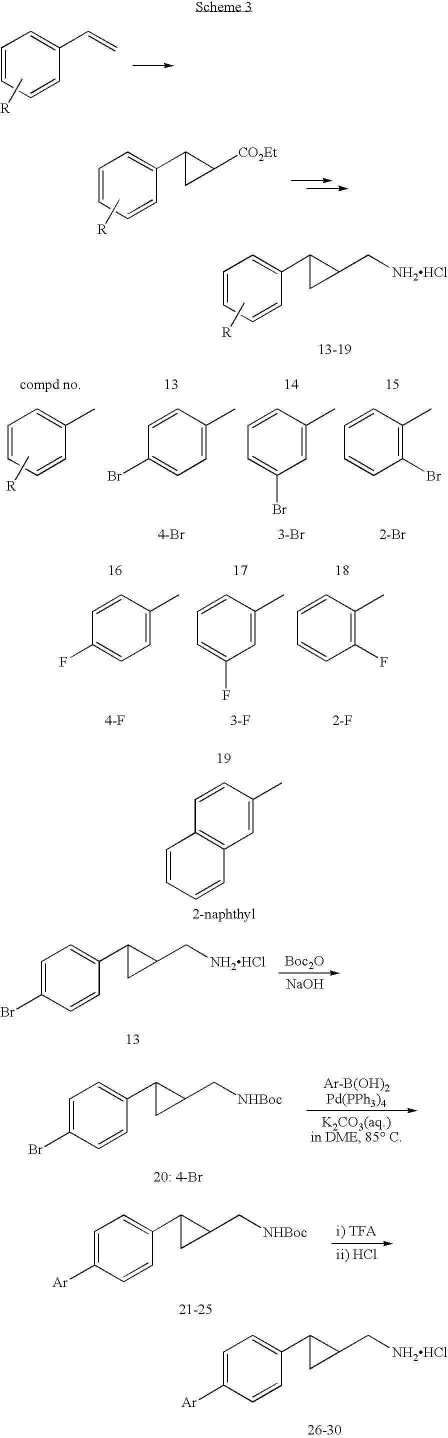

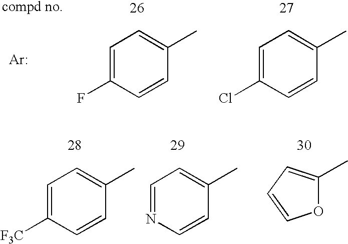

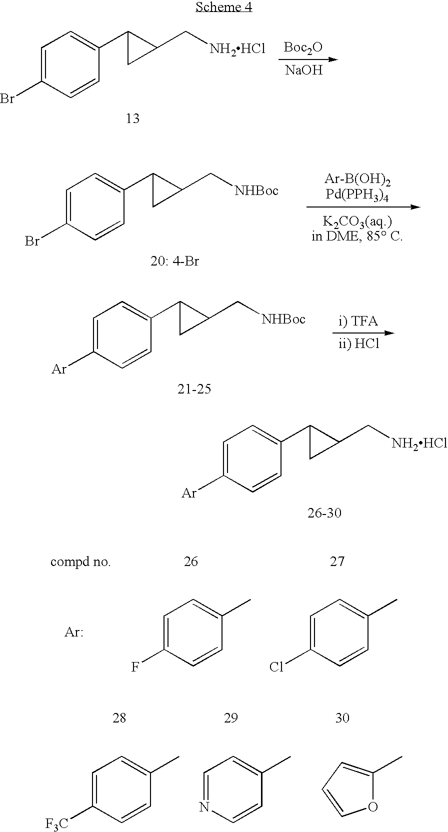

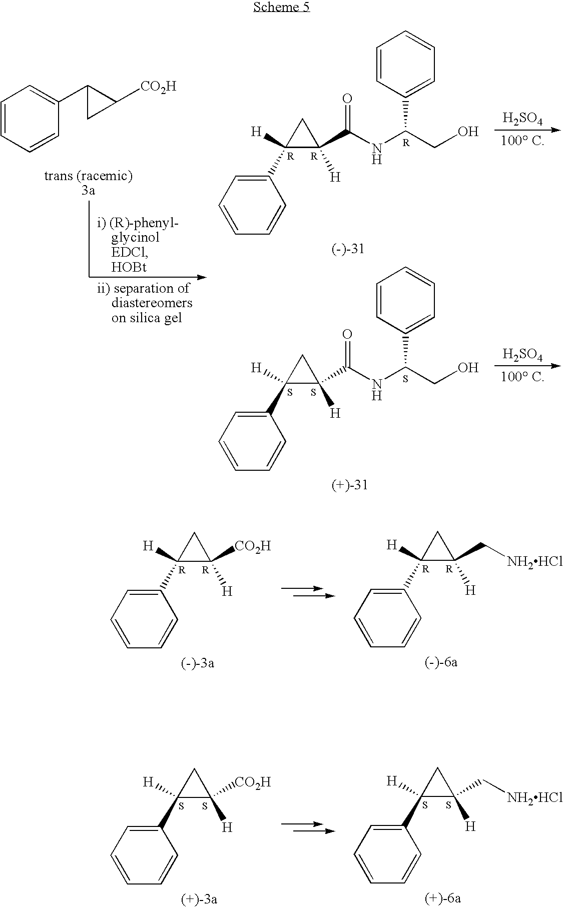

Cyclopropane ring-substitution can, for example, be accomplished by starting from an appropriately functionalized trisubstituted olefin as illustrated in Scheme 7. A Knoevenagel condensation between a substituted benzaldehyde and dimethylmalonate is carried out (Rappoport, Z., Gazit, (1986) J. Org. Chem., 51, 4107-4111) After cyclopropanation of the benzylidenemalonate (Lorenz, J. C., Long et al. (2004) J. Org. Chem., 69, 327-334), the sterically more accessible trans ester group is selectively hydrolyzed with methanolic KOH. The carboxyl group is then converted to the amine through a sequence of steps involving reduction to alcohol, conversion of the alcohol to a leaving group, followed by azide displacement and reduction. From this amine, various other derivatives are prepared by using the remaining ester group. The ester can be transesterified to form other esters, converted to the metabolically more stable amide derivatives using various amines, or reduced to alcohol to allow for ether synthesis. Additionally, the OH group of this alcohol can be removed using the Barton deoxygenation reaction, or this OH group activated to allow for organocuprate coupling reactions to introduce other alkyl groups.

Scheme 8 illustrates another method for preparation of substituted cyclopropane derivatives. Such analogs can be readily prepared starting from the appropriate α-substituted styrene derivative, such as the methyl or protected hydroxymethyl compounds. These intermediates are cyclopropanated as described herein and the ester converted to the amine. In the case of the hydroxymethyl compound illustrated in Scheme 8, additional derivatives are prepared, for example, by oxidation of the alcohol to an aldehyde followed by Wittig chemistry which allows for introduction of a variety of more rigid, unsaturated hydrocarbon chains, which can be converted to various alkenyl derivatives of either cis or trans olefin stereochemistry.

An additional example of synthesis of substituted cyclopropane derivatives is illustrated in Scheme 9. In this case, an appropriate substituted styrene is employed as the starting material. This method is particularly useful for halogen substitution.

Another exemplary method of preparing substituted cyclopropane derivatives is illustrated in Scheme 10. In this case, the cyclopropanes are prepared

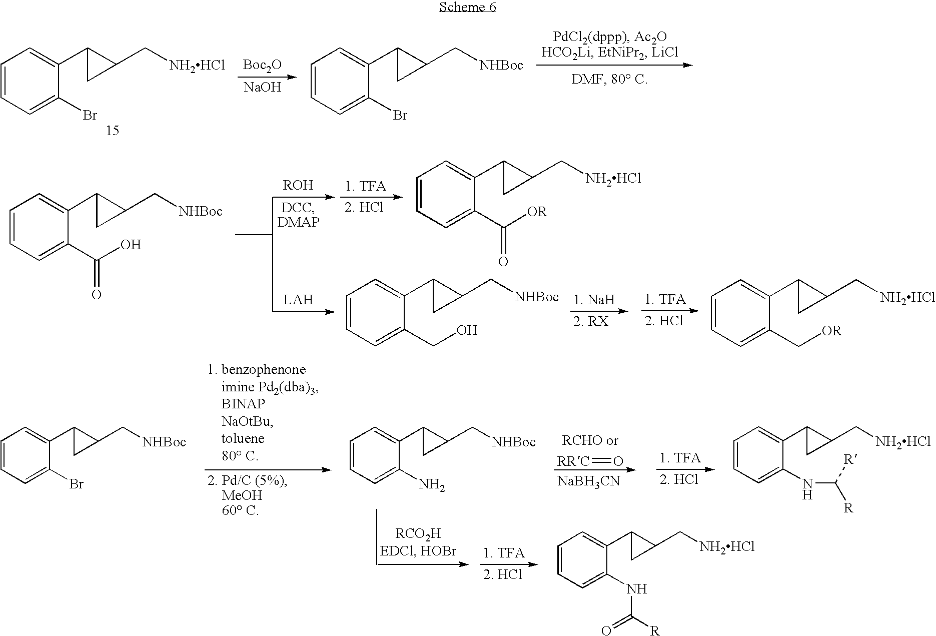

from 1,1-disubstituted alkenes. This method is particularly useful for preparation of cyclopropane with additional aryl group substitution, as illustrated.

Aryl ring substituents can also be added employing the Sonogashira coupling reaction as illustrated in Scheme 11 which is particularly useful for introducing acetylenic groups (DeVasher, R. B., Moore, L. R., Shaughnessy, K. H. Aqueous-phase, palladium-catalyzed cross-coupling of aryl bromides under mild conditions, using water-soluble, sterically demanding alkylphosphines. J. Org. Chem., 69, 7919-7927, (2004). Functionalization can be carried out at the ortho, meta, or para positions.

An alternative method for preparation of substituted arylcyclopropanes, illustrated in Scheme 12, employs coupling of reactive cyclopropyl-boron compounds under palladium catalysis with various arylbromides or iodides. Substituted phenyl halides are coupled with the reactive potassium cyclopropyl trifluoroborate under the Suzuki coupling conditions. The potassium cyclopropyl trifluoroborate is prepared as follows. First, the trans-alkenylboronic acid is prepared by the coupling of an N-protected propargyl amine with a bisalkylborane. After protection of the resulting boronic acid with pinacol, cyclopropanation of the alkenylboronic ester followed by in situ treatment with excess KHF2 gives the corresponding potassium cyclopropyl trifluoroborate. This intermediate is then subjected to the Pd-based coupling reaction with an aryl bromide or iodide. The method of Scheme 12 can be used to introduce a hydroxyl group at either the ortho, meta, or para positions of the aromatic ring.

Biphenyl and heteroaryl-phenyl derivatives are prepared by employing them as reaction partners in the Suzuki coupling reaction (Scheme 13). Many boronic acids are now available commercially.

Analogous methods are used to prepare cyclopropanes linked to heterocyclic moieties. The selected heteroaryl bromides are coupled with the cyclopropyl trifluoroborate as illustrated in Scheme 14.

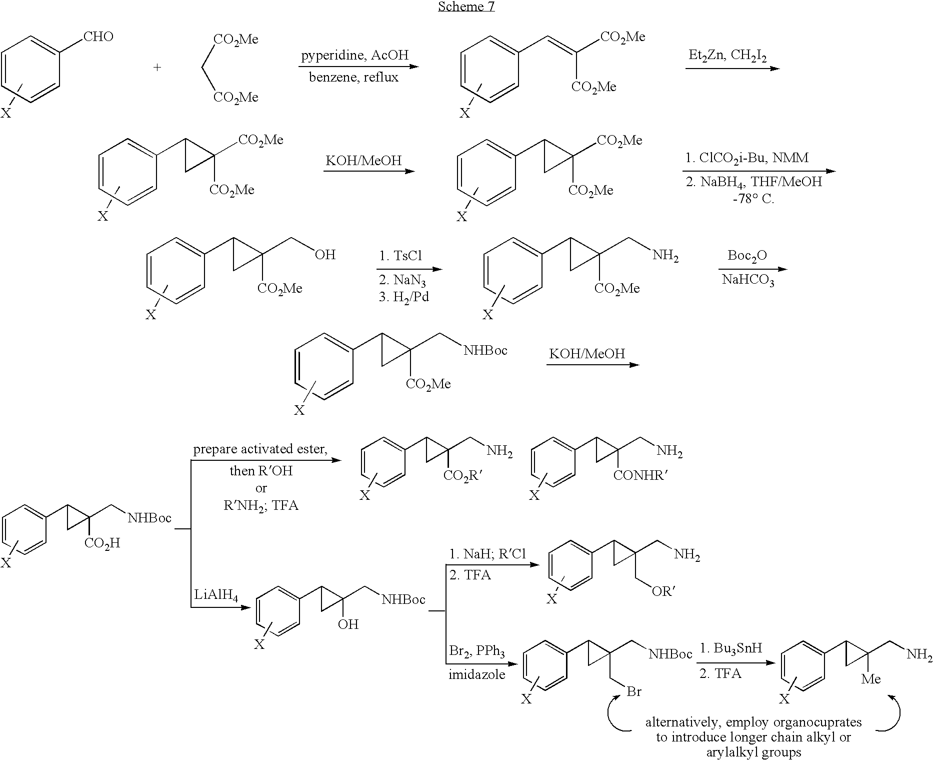

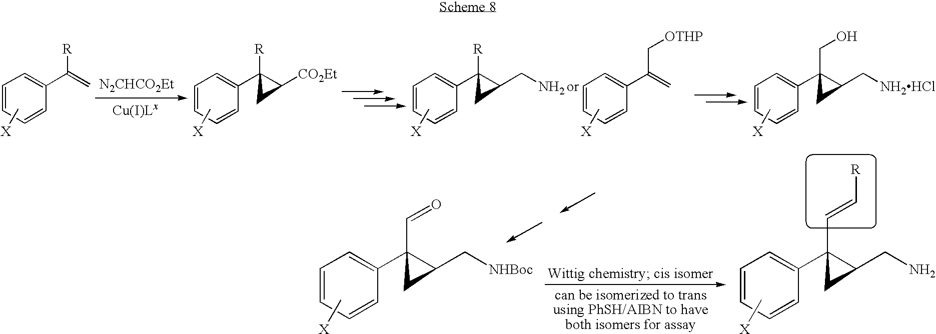

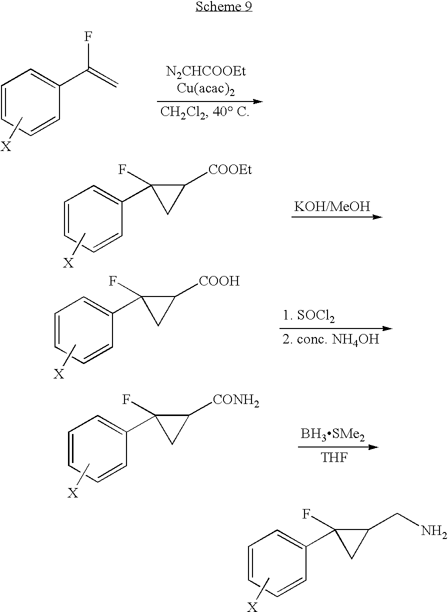

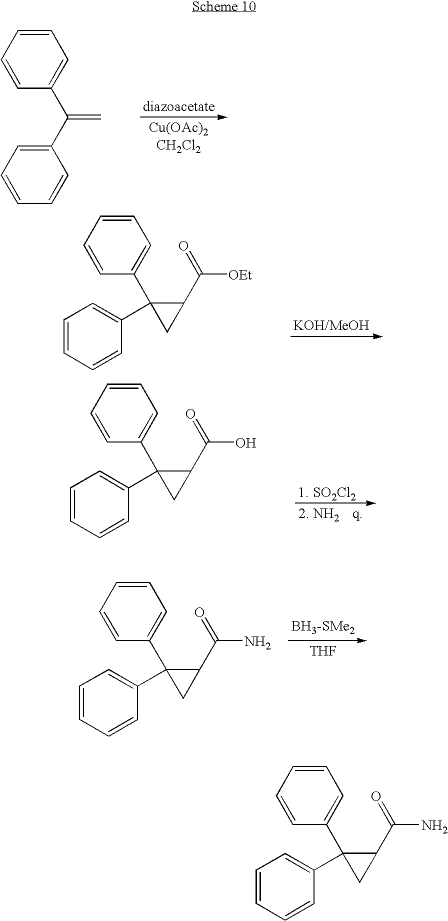

Additional synthetic methods that can be employed in the preparation of compounds of this invention can be found in published international application WO 2005/007614, published Jan. 27, 2005.

### Biological Activity

Compounds of the invention function generally as modulators (agonists, partial agonist, antagonists, partial antagonists as well as selective agonists) of 5-HT receptors of the 5-HT2 family. More specifically, compounds of the invention function as agonists of 5-HT2 receptors. Even more specifically, compounds of the invention function as agonists or selective agonists of the 5-HT2C receptors. Compounds of the invention are useful in prevention or treatment of diseases, conditions, disorders or treatment or amelioration of undesired symptoms associated with the 5-HT2 family of receptors. More specifically, the compounds of the invention can be used in prevention or treatment of diseases, conditions, disorders or treatment or amelioraton of undesired symptoms associated with the 5-HT2C receptor.

Diseases, conditions, disorders symptoms associated with the 5-HT2C receptor include among others, obesity, eating disorders, diabetes, cardiovascular disorders, sleep disorders (e.g., sleep apnea), disorders of the central nervous system, damage to the central nervous system; gastrointestinal disorders, depression, atypical depression, bipolar disorders, anxiety disorders, obsessive-compulsive disorders, social phobias or panic, sexual dysfunction, psychoses, schizophrenia, migraine, other conditions associated with cephalic pain or other pain, raised intracranial pressure, epilepsy, personality disorders, Alzheimer's disease, age-related behavioral disorders, behavioral disorders associated with dementia, organic mental disorders, mental disorders in childhood, aggressivity, age-related memory disorders, chronic fatigue syndrome, drug and alcohol addiction, bulimia, anorexia nervosa and premenstrual tension.

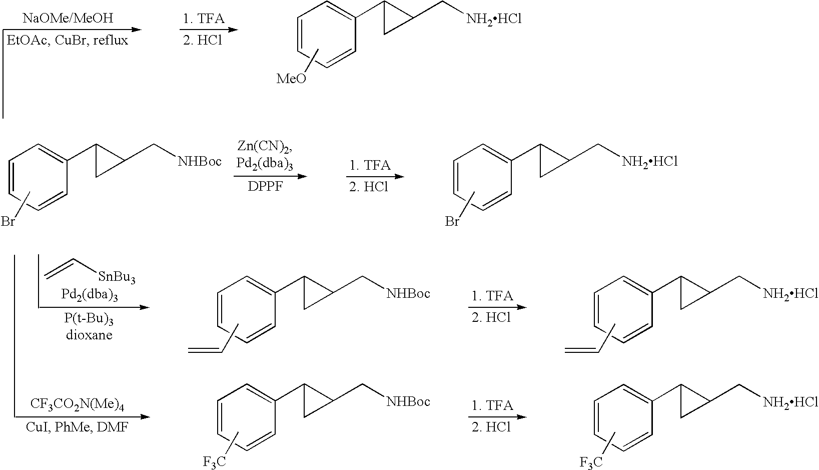

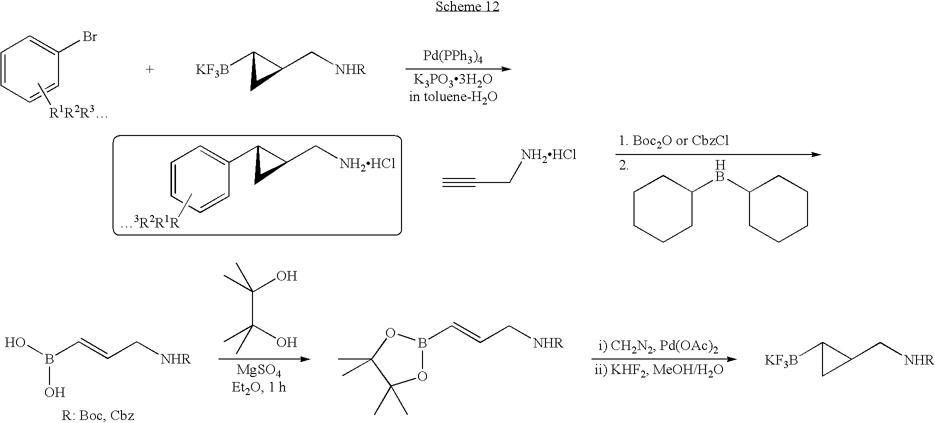

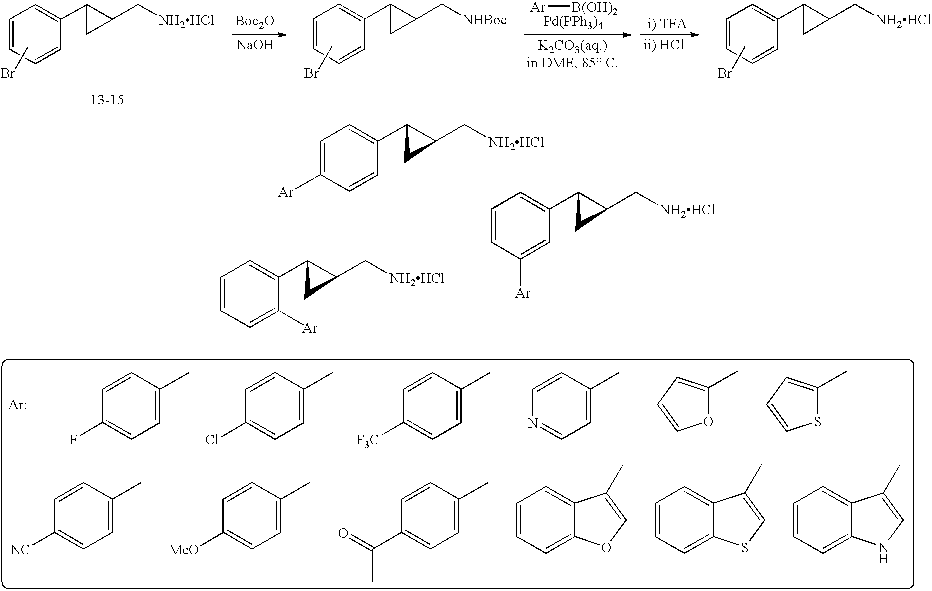

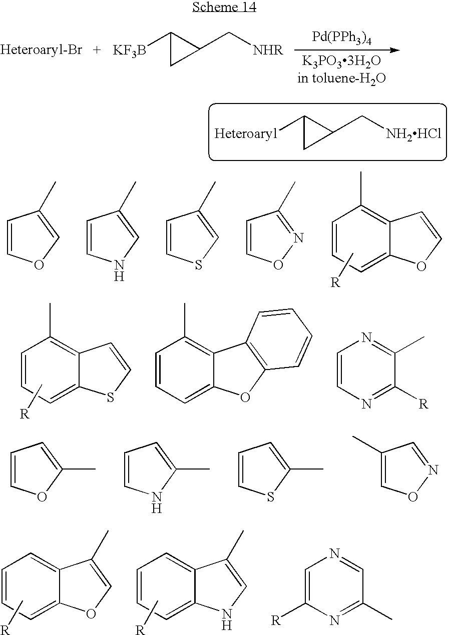

The functional activity of the compounds for the human 5-HT2A, human 5-HT2B, and human 5-HT2C receptors is determined by measurement of Ca++-flux as detailed in the Examples, in transiently transfected HEK-293 cells and the results are summarized in Table I. Structures of compounds listed in Table I are given in Scheme 15.

Table 1 also provides calculated log P values for the listed compounds. The log P value of a compound is the logarithm of its partition coefficient between n-octanol and water log(coctanol/cwater). This value is a measure of the hydrophilicity of a compound. Low hydrophilicities (high log P values) are associated with poor absorption or permeation. Compounds having log P values of 5 or less are more preferred for drug applications. Values listed in Table 1 were calculated employing a program “C log P” available from Daylight Chemical Information Systems, Inc., Aliso Viejo, Ca. In Table 1 N/A means not active.

trans-6a (TKU-II-17 (±)-trans) shows more potent agonist activity than tranylcypromine (1) at the 5-HT2C receptor. The increased distance between the amino group and the aryl ring as well as the greater flexibility of 6a may allow for a better interaction with the 5-HT2C receptors, in comparison to 1. IN contrast, the cis isomer cis-6b (TKU-II-19 (±)-cis) is poorly active at all three of the 5-HT2 receptors. There is a strong preference for trans stereochemistry about the cyclopropane ring in order to optimize 5-HT2C receptor activity. In general, the introduction of substituents on the amine nitrogen led to a reduction in activity in comparison to the parent compounds. These results indicate that an unsubstituted NH2 group is preferred for high potency at the 5-HT2C receptor. Among the bromophenyl derivatives 13-15, both the o-bromo and m-bromo analogs 14 and 15 showed improved agonist activity at the 5-HT2C receptor compared to the parent structure; moreover, ligand 15 also shows a much improved selectivity for the 5HT2C receptor relative to the 5-HT2B receptor. Interestingly, compounds 14 and 15 show partial agonist activity at the 5-HT2C receptor (Emax 58% and 75%, respectively). The o- and p-fluoro substituted ligands, 18 and 16, also show good potency at the 5HT2C receptor.

In the case of the analogs bearing an additional aryl or heteroaryl ring at the p-position of the phenyl ring, both the p-chlorobiphenyl analog 27 and the p-furanylphenyl analog 30 exhibited good selectivity over the 5-HT2A receptor. In addition, compound 30 also shows no activity at the 5HT2B receptor.

In vivo Activity in a Seizure Model: Compound 6a was tested in the 6 Hz seizure model in mice at a dose of 100 mg/kg administered i.p. The compound was found to cause minimal motor impairment at early time points (15-60 minutes after dosing). After four hours, three of the four animals tested were protected from seizure, suggesting that perhaps delayed absorption or a metabolite may be responsible for the observed protection. While signs of some early toxicity were noted in the i.p. mouse screens, the preliminary data indicate that compounds of this invention have potential use in the treatment of epilepsy. These results clearly demonstrate that cyclopropane compounds of this invention are able to penetrate the brain-blood barrier (BBB) and to exert a desirable biological effect.

TABLE 1
Functional Activity and Selectivity (EC50 [nM])

| | | 5-HT2A | 5-HT2B | 5-HT2C | | |
|---|---|---|---|---|---|---|
| | logP | human | human | human | 2A/2C | 2B/2C |
| Compound | ‘Calc.’ | EC50 [nM] | | | ratio | |
| Tranylcypromine (1) | 1.48 | | | 271 | | |
| TKU-II-17 (±)-trans (6a) | 1.72 | 49.7 | 18.6 | 9.6 | 5.18 | 1.94 |
| TKU-II-19 (±)-cis (6b) | 1.72 | 705 | 308 | 152 | 4.64 | 2.03 |
| TKU-II-113 (+)-trans | 1.72 | 44.3 | 35.3 | 4.5 | 9.84 | 7.84 |
| TKU-II-115 (−)-trans | 1.72 | 1685 | 1537 | 280 | 6.02 | 5.49 |
| TKU-II-27 | 2.16 | NA (not | NA | NA | | |
| | | active) | | | | |
| TKU-II-39 | 1.86 | 3714 | 979.5 | 717.6 | 5.18 | 1.36 |
| TKU-II-36 | 3.89 | 123328 | 1019000 | 7458 | 16.5 | 137 |
| TKU-II-42 (19) | 2.89 | 951.4 | 15444 | 6749 | 0.14 | 2.29 |
| TKU-II-30 | 3.65 | 9789000 | 10740000 | 10531 | 929.5 | 1020 |
| TKU-II-38 | 1.62 | 164665 | NA | 2501 | 65.8 | |
| TKU-II-33 | 2.46 | 1468000 | NA | 8838 | 166 | |
| | ClogP | 5-HT2A | 5-HT2B | 5-HT2C | | |
| Compound | ‘Daylight’ | human | human | human | 2A/2C | 2B/2C |
| TKU-II-44 (13) | 2.58 | 1061 | 495.2 | 179.9 | 5.90 | 2.75 |
| TKU-II-57 (26) | 3.75 | 1034000 | 623945 | 10231 | 101.1 | 61 |
| TKU-II-58 (28) | 4.49 | 3411 | 1269 | 226.0 | 15.1 | 5.62 |
| TKU-II-60 (29) | 2.11 | NA | NA | NA | | |
| | | | | (Not converge) | | |
| TKU-II-71 (±)-trans (15) | 2.58 | 63.5 | 19.2 | 6.5 | 9.8 | 2.9 |
| TKU-II-153-1 (+)-trans | 2.58 | 48.7 | 21.3 | 11.4 | 4.3 | 1.8 |
| TKU-II-153-2 (−)-trans | 2.58 | 2271 | 251 | 307 | 7.4 | 0.82 |
| TKU-II-81 (30) | 2.99 | 281310 | NA | 82 | 3431 | |
| TKU-II-78 (14) | 2.58 | 29.5 | 94 | 1.8 | 16.4 | 52.2 |
| TKU-II-82 (27) | 4.32 | 199137 | 3540 | 429 | 464 | 8.3 |
| TKU-II-90 (16) | 1.86 | 7.1 | 7.1 | 7.4 | 0.96 | 0.96 |
| TKU-II-99 (17) | 1.86 | 63.4 | 4681 | 63.2 | 1.0 | 74 |
| TKU-II-100 (18) | 1.86 | 0.62 | 0.2 | 0.66 | 0.94 | 0.3 |
| TKU-II-116 | 4.32 | 3996 | | 123.9 | 32.3 | |
| TKU-II-117 | 4.49 | 277288 | | 465.3 | 596 | |
| TKU-II-119 | 4.32 | 215.4 | 63.7 | 2.54 | 85 | 25 |
| | logP | 5-HT2A | 5-HT2B | 5-HT2C | | |
| Compound | ‘Daylight’ | human | human | human | 2A/2C | 2B/2C |
| TKU-II-120 | 4.49 | Not | Not | 10.1 | | |
| | | converge | converge | | | |
| TKU-II-191 | 3.75 | | | | | |
| TKU-II-192 | 3.04 | | | | | |
| TKU-II-194 | 4.32 | | | | | |
| TKU-II-195 | 4.32 | | | | | |
| TKU-II-177 | 2.00 | | | | | |
| TKU-II-197 | 2.23 | | | | | |
| TKU-II-212 | 0.74 | | | | | |
| TKU-II-210 | 2.66 | | | | | |
| TKU-II-211 | 1.33 | | | | | |
| TKU-II-216 | 4.38 | | | | | |
| TKU-II-217 | 4.08 | | | | | |
| TKU-II-233 | 2.79 | | | | | |
| TKU-II-235 | 3.19 | | | | | |
| TKU-II-242 | −0.16 | | | | | |
| TKU-II-241 | 3.75 | | | | | |

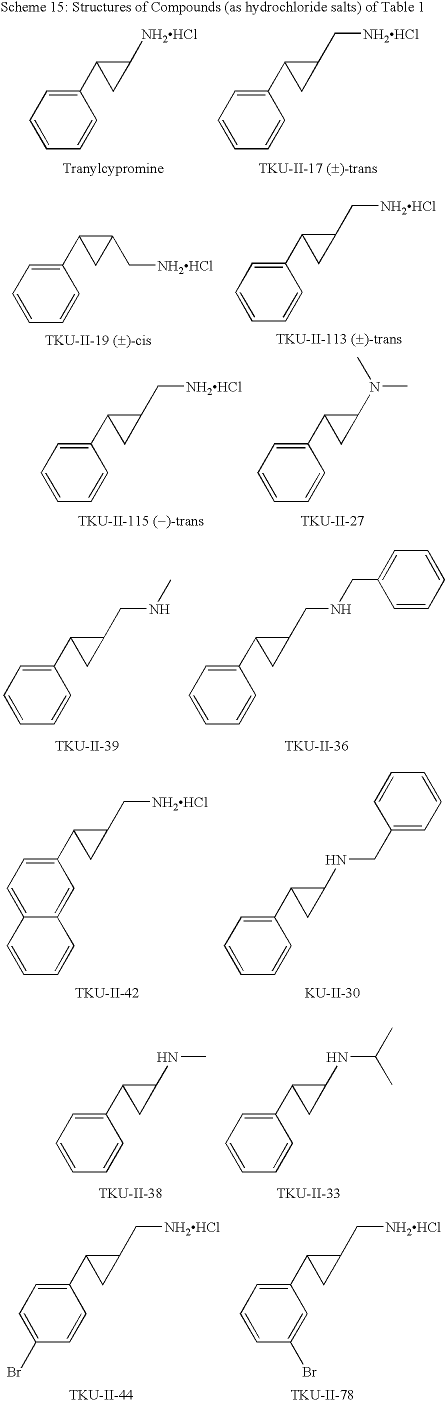

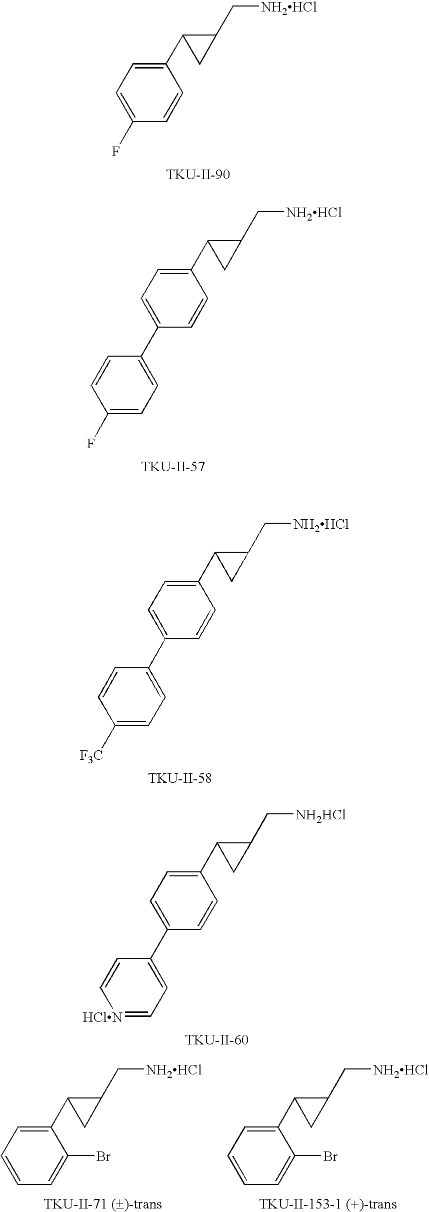

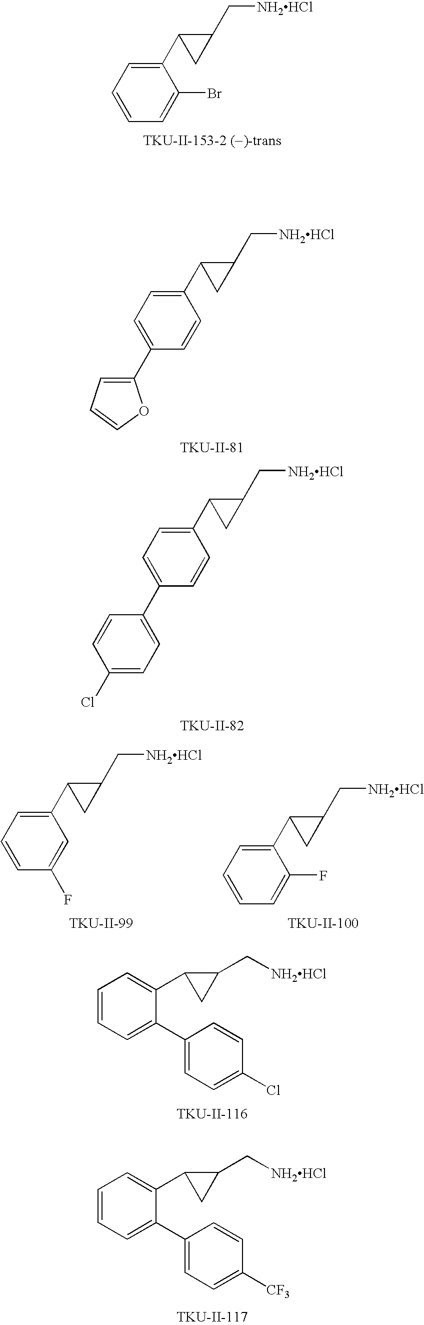

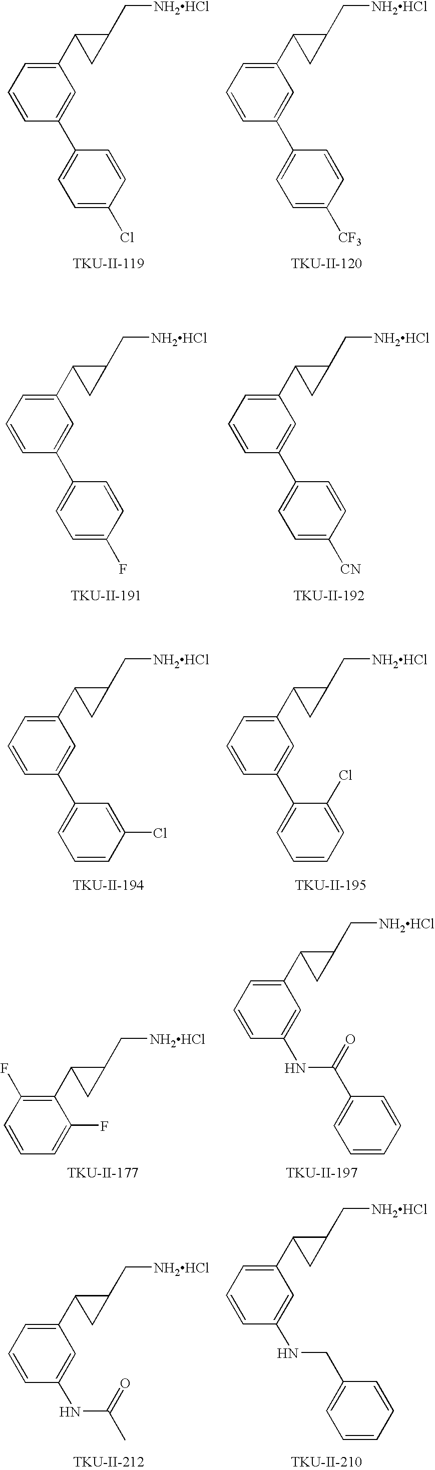

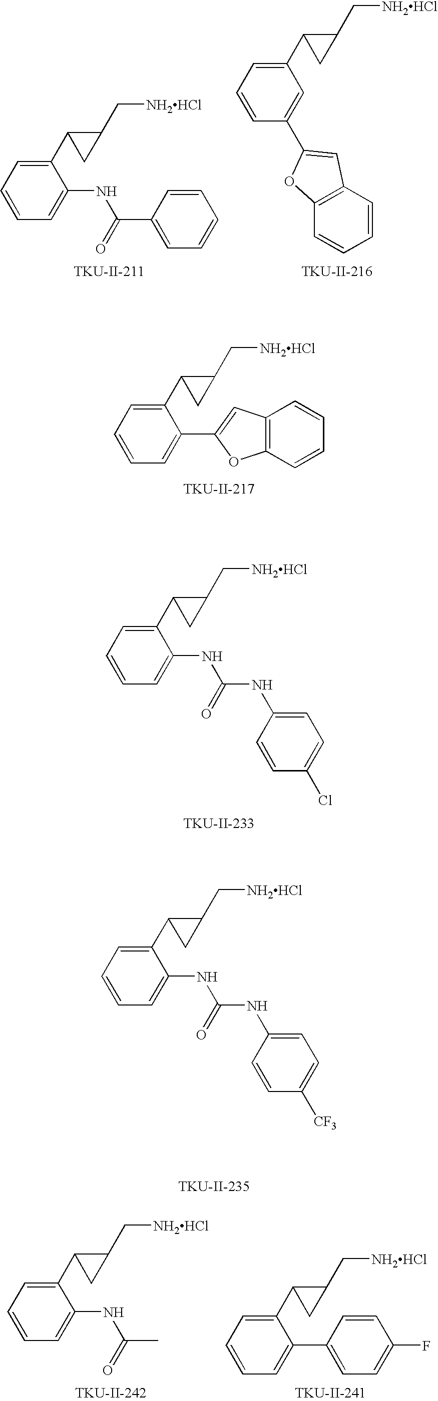

The compounds, salts, esters, and solvates thereof of this invention have utility as 5-HT2C receptors agonists and selective agonist and may also exhibit agonist activity for 5-HT2A receptors. Compounds of this invention are useful for activating a 5-HT2C receptor in vivo or in vitro wherein the receptor is contacted with an amount of the compound that is effective for activating the receptor under selected conditions. The amount of a given compound that is effective for such activation can be readily assessed in view of the teachings herein and what is known in the art.

The compounds, pharmaceutically acceptable salts, esters, solvates and prodrugs of this invention generally have therapeutic activity for treatment or prophylaxis of any condition, disorder, disease or undesired symptom that is recognized in the art to be associated with a 5-HT2C receptor.

5-HT2C receptor-associated disorders, conditions, diseases and symptoms include among others: obesity, eating disorders (e.g., hyperphagia, bulimia or anorexia nervosa), gastrointestinal disorders, malfunction of gastrointestinal motility, diabetes, sleep disorders, sleep apnea, hypertension, hypertension, hyperlipidemia, cardiovascular disease, central nervous system disorders, damage to the central nervous system associated with trauma, stroke, or spinal cord injury or complications, psychiatric disorders, obsessive-compulsive disorder, anxiety, panic disorder, schizophrenia, schizoaffective disorder, schizophreniform disorder, L-DOPA-induced psychosis, psychosis, dementia, memory deficit, intellectual deficit associated with Alzheimer's disease, bipolar disorders, adjustment disorders, depression, movement disorders, dystonia, chronic pain, Parkinson's Disease, or Alzheimer's Disease, sexual dysfunction in males or females, erectile dysfunction, epilepsy, headache and migraines. 5-HT2C receptor agonists are particularly useful for treatment of obesity and the comorbidities thereof including Type II diabetes, cardiovascular disease, hypertension, hyperlipidemia, stroke, osteoarthritis, sleep apnea, gall bladder disease, gout, some cancers, some infertility, and early mortality.

5-HT2C receptor agonist are also useful in the methods of decreasing food intake in an individual, of inducing satiety in an individual, of controlling weight gain of an individual and in generally providing benefit to individuals in the form of weight reduction.

The present invention provides methods of treating disorders, diseases conditions and symptoms in a mammal and particularly in a human, by administering to an individual in need of treatment or prophylaxis, a therapeutically effective amount of a compound of this invention to the mammal in need thereof. The result of treatment can be partially or completely alleviating, inhibiting, preventing, ameliorating and/or relieving the disorder, condition or one or more symptoms thereof. Administration includes any form of administration that is known in the art to be effective for a given type of disease or disorder, is intended to encompass administration in any appropriate dosage form and further is intended to encompass administration of a compound, pharmaceutically acceptable salt, solvate or ester thereof alone or in a pharmaceutically acceptable carrier thereof or administration of a prodrug derivative or analog of a compound of this invention which will form an equivalent amount of the active compound or substance within the body. An individual in need of treatment or prophylaxsis includes those who have been diagnosed to have a given disorder or condition and to those who are suspected, for example, as a consequence of the display of certain symptoms, of having such disorders or conditions.

The term “therapeutically effective amount,” as used herein, refers to the amount of a compound of Formula I that, when administered to an individual is effective to at least partially treat a disorder, disease or condition from which the individual is suffering, or to at least partially ameliorate a symptom of such disorder, disease or condition. As is understood in the art, the therapeutically effective amount of a given compound will depend at least in part upon, the mode of administration, any carrier or vehicle (e.g., solution, emulsion, etc.) employed, the specific disorder or condition, and the specific individual to whom the compound is to be administered (age, weight, condition, sex, etc.).

The dosage requirements need to achieve the “therapeutically effective amount” vary with the particular compositions employed, the route of administration, the severity of the symptoms presented and the particular subject being treated. Based on the results obtained in standard pharmacological test procedures, projected daily dosages of active compound can be determined as is understood in the art.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like.

In addition these salts may be prepared from addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polymine resins and the like. Compounds of formula I can also be present in the form of zwitterions.

The invention expressly includes pharmaceutically usable solvates of compounds according to Formula I. The compounds of Formula I can be solvated, e.g., hydrated. The solvation can occur in the course of the manufacturing process or can take place, e.g., as a consequence of hygroscopic properties of an initially anhydrous compound of Formula I (hydration).

“Pharmaceutically acceptable esters” refers ester derivatives of compounds of Formula I formed at certain functional groups which are capable of conversion back to the parent compounds in vivo. For example, the COOH groups of compounds can be esterified. Examples of such esters include physiologically acceptable and metabolically labile ester derivatives, such as methoxymethyl esters, methylthiomethyl esters and pivaloyloxymethyl esters. Additionally, any physiologically acceptable equivalents of the compounds of general formula I, similar to the metabolically labile esters, which are capable of producing the compounds of general formula I in vivo, are encompassed within this invention. Esters more specifically include methyl, ethyl, propyl, butyl and benzyl esters. Further examples of pharmaceutically useful esters are compounds of Formula I, wherein hydroxy groups can be esterified, for example by formation of formate, acetate, propionate, butyrate, isobutyrate, valerate, 2-methylbutyrate, isovalerate and N,N-dimethylaminoacetate esters.

In certain embodiments, the present invention is directed to prodrugs of compounds of Formula I. The term “prodrug,” as used herein, means a compound that is convertible in vivo by metabolic means (e.g. by hydrolysis) to a compound of Formula 1. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al. (ed.), “Design and Application of Prodrugs, Textbook of Drug Design and Development, Chapter 5, 113-191 (1991), Bundgaard, et al., Journal of Drug

Delivery Reviews, 8:1-38 (1992), Bundgaard, J. of Pharmaceutical Sciences, 77:285 et seq. (19SS); and Higuchi and Stella (eds.) Prodrugs as Novel Drug Delivery Systems, American Chemical Society (1975), each of which is hereby incorporated by reference in its entirety.

The compounds of this invention can be administered in oral dosage forms including tablets, capsules, pills, powders, granules, elixirs, tinctures, suspensions, syrups and emulsions. Oral dosage forms may include sustained release or timed release formulations. The compounds of this invention may also be administered intravenously, intraperitoneally, subcutaneously, or intramuscularly, all using dosage forms well known to those of ordinary skill in the pharmaceutical arts. Compounds of the invention can further be administered topically employing appropriate carriers.

Compounds of this invention can also be administered in intranasal form by topical use of suitable intranasal vehicles. For intranasal or intrabronchial inhalation or insulation, the compounds of this invention may be formulated into an aqueous or partially aqueous solution, which can then be utilized in the form of an aerosol.

The compounds of this invention can also be administered to the eye, preferrably as a topical opthalmic formulation. The compounds of this invention can also be combined with a preservative and an appropriate vehicle such as mineral oil or liquid lanolin to provide an opthalmic ointment. The compounds of this invention may be administered rectally or vaginally in the form of a conventional suppository. The compounds of this invention may also be administered transdermally through the use of a transdermal patch containing the active compound and a carrier that is inert to the active compound, is non toxic to the skin, and allows delivery of the agent for systemic absorption into the blood stream via the skin.

The compounds of the invention may be administered employing an occlusive device. A variety of occlusive devices can be used to release an ingredient into the blood stream such as a semipermeable membrane covering a reservoir containing the active ingredient with or without a carrier, or a matrix containing the active ingredient. Other occlusive devices are known in the literature.

The therapeutically active compounds of the invention can be administered alone, but generally will be administered with a pharmaceutical carrier selected upon the basis of the chosen route of administration and standard pharmaceutical practice.

Pharmaceutical compositions of this invention comprise one or more compounds, pharmaceutically acceptable salts, esters or solvates thereof or a prodrug thereof in combination with a pharmaceutically acceptable carrier, excipient, or diluent. Such compositions are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety.

Pharmaceutically acceptable carriers are those carriers that are compatible with the other ingredients in the formulation and are biologically acceptable. Carriers can be solid or liquid.

Solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water of appropriate purity, aqueous solutions (particularly containing additives as above, e.g. cellulose derivatives, sodium carboxymethyl cellulose solution), alcohols (including monohydric alcohols and polyhydric alcohols e.g. glycols) and their derivatives, and oils. For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant. Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The carrier can also be in the form of creams and ointments, pastes, and gels. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable.

Preferably, the pharmaceutical composition is in unit dosage form, e.g. as tablets or capsules. In such form, the composition is sub-divided in unit dose containing appropriate quantities of the active ingredient; the unit dosage forms can be packaged compositions, for example, packaged powders, vials, ampules, pre-filled syringes or sachets containing liquids. The unit dosage form can be, for example, a capsule or tablet itself, or it can be the appropriate number of any such compositions in package form.

The dosage can vary within wide limits and as is understood in the art will have to be adjusted to the individual requirements in each particular case as discussed above. By way of general guidance, the daily oral dosage can vary from about 0.01 mg to 1000 mg, 0.1 mg to 100 mg, or 10 mg to 500 mg per day of a compound of Formula I or of the corresponding amount of a pharmaceutically acceptable salt thereof. The daily dose may be administered as single dose or in divided doses and, in addition, the upper limit can also be exceeded when this is found to be indicated.

Certain compounds of this invention also have utility as starting materials for the preparation of compounds that are in turn useful in various therapeutic applications, for example, for the preparation of additional 5-HT2C receptor agonists or selective antagonists.

The scope of the invention as described and claimed encompasses the racemic forms of the compounds as well as the individual enantiomers and non-racemic mixtures thereof. The compounds of the invention may contain one or more asymmetric carbon atoms, so that the compounds can exist in different stereoisomeric forms. The compounds can be, for example, racemates or optically active forms. The optically active forms can be obtained by resolution of the racemates or by asymmetric synthesis. In a preferred embodiment of the invention, enantiomers of the invention exhibit specific rotation [α] that is + (positive). Preferably, the (+) enantiomers are substantially free of the corresponding (−) enantiomer. Thus, an enantiomer substantially free of the corresponding enantiomer refers to a compound which is isolated or separated via separation techniques or prepared free of the corresponding enantiomer. “Substantially free,” means that the compound is made up of a significantly greater proportion of one enantiomer. In preferred embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N.Y., 1962); Wilen, S. H. Tables of Resolving Agents and Optical Resolutions p. 268 (E. L. Eliel, Ed., Univ. of Notre Dame Press, Notre Dame, Ind. 1972).

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. A number of specific groups of variable definitions have been described herein. It is intended that all combinations and subcombinations of the specific groups of variable definitions are individually included in this disclosure. When a compound is described herein such that a particular isomer, enantiomer or diastereomer of the compound is not specified, for example, in a formula or in a chemical name, that description is intended to include each isomers and enantiomer of the compound described individual or in any combination.

Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Isotopic variants may also be useful in diagnostic assays and in therapeutics. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

Any one or more of the compounds specifically disclosed in this specification can be excluded from any of the embodiments of the invention. Any one or more disorder, conditions or disease, specifically disclosed in this specification can be excluded from any of the embodiments of the invention.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, can be excluded from the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, carriers, dosage forms, methods of administration, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference to the extent that there is no inconsistency with the disclosure of this specification. Some references provided herein are incorporated by reference to provide details concerning sources of starting materials, additional starting materials, additional reagents, additional methods of synthesis of the compounds herein, additional methods of analysis and assessment of the biological functions of the compounds herein, additional biological materials, methods for assessing biological function of the compounds herein and additional therapeutic and prophylactic uses of the 5-HT2C receptor agonists of this invention.

### THE EXAMPLES

### Biological Methods

The functional activity of compounds for the human 5-HT2A, human 5-HT2B, and human 5-HT2C receptors as agonists, partial agonists and antagonists is determined by measurement of Ca++-flux. The results are summarized in Table 1 where EC₅₀ for different receptors are given (nM) and the ratio of activities 2A/2C and 2B/2C are also given. As noted above, other methods for assessing agonist/antagonist function of compounds for 5-HT2 family receptors are known in the art.

Standard methods are employed for 5-HT assays (See: Roth, B. L et al. Neuropharmacology, 23, 1223-1225, (1984); Roth, B. L., et al. J Neurochem, 49, 1833-1838, (1987); Roth, B. L., et al. J Pharmacol Exp Ther, 268, 1403-1410, (1994); Gray, J. A, et al. Biochemistry, 42, 10853-10862, (2003); Roth, B. L, et al. Nat Rev Drug Discov, 3, 353-359, (2004); and Elphick, G. F., et al. Science, 306, 1380-1383, (2004)).

For these studies stably expressing lines of h5-HT2A, h5-HT2B, and h5-HT2C receptors are prepared in HEK-293 cells. For the h5-HT2A and h5-HT2C-INI receptors, stably expressing HEK-293 cell lines were constructed as previously described using a pIRES-NEO vector system (See: Rauser et al, J Pharmacol Exp Ther 299: 83-89, 2001).

For the h5-HT2B receptors, cDNAs are subcloned into pBABEPuro and high titer recombinant retroviral stocks made using standard procedures under appropriate containment conditions. HEK-293 cells are infected and then selected with 5 microg/ml puromycin, and the surviving recombinant clones expanded and selected for high levels of h5-h5-HT2B expression via radioligand binding using [3H]LSD (Setola, V, et al. Mol Pharmacol, 63, 1223-1229, (2003)), respectively.

pBabepuro-FLAG-h5-HT2BR was created by PCR amplification using human 5-HT_{2B}R DNA as template (Rothman et al., 2000). This PCR product contained h5-HT_{2B}R flanked 5′ by sequence for a BamHI site, a cleavable membrane insertion signal peptide (MKTIIALSYIFCLVFA derived from influenza hemagglutinin (Guan et al., 1992)) followed immediately by a FLAG epitope (DYKDDDDK) in frame with the h5-HT_{2B}R start site and following the stop codon flanked 3′ by sequence for a SalI site. This product was digested with BamHI and SalI and inserted into the same sites of pBabepuro (Morgenstern and Land, 1990). The resulting construct was verified to be correct by automated sequencing (Cleveland Genomics; Cleveland, Ohio). Amphotropic retrovirus was produced by co-transfecting a 6 cm plate of Human Embryonic Kidney (HEK) 293TS cells (provided by C. M. Counter, Duke University; Durham, N.C.) with 1 μg of desired construct (e.g., pBabepuro-FLAG-h5-HT_{2B}R) and 1 μg of the amphotropic packaging plasmid pCL-10A1 (Imgenex; San Diego, Calif.) using FuGene6 (Roche; Indianapolis, Ind.) transfection reagent. Virus-containing medium was collected between 24-60 hrs post-transfection, filtered with a sterile 0.45 μm filter and supplemented with a final concentration of 4 μg/ml polybrene (Sigma-Aldrich) and incubated with HEK293 cells (ATCC: CRL-1573; Manassas, Va.) for 6-12 hrs to infect. 36-48 hrs post-infection HEK293 cells were selected, and subsequently grown, in DMEM supplemented with 10% Fetal bovine serum (FBS), 1 mM sodium pyruvate and 2 μg/ml puromycin. The first confluent plate under selection was designated as population doubling (pd) 0. All assays performed utilized FLAG-h5-HT_{2B} polyclonal HEK293 cell line between 5 and 20 pds. All cells were grown at 37° C. in 5% CO₂ atmosphere. Rat 5-HT2A, and 5-HT_{2C} receptor 3T3 mouse fibroblast cell lines have been previously described (Shapiro et al., 2003) and were cultured in DMEM supplemented with 10% FBS, 1 mM sodium pyruvate and 600 μg/ml G418.

### Intracellular Calcium Mobilization Assay

Stable cell lines were plated with ˜40,000 cells/well into uncoated (3T3 cells) or poly-L-lysine coated (HEK293 cells) 96-well plates in DMEM supplemented with 5% dialyzed FBS. 16-24 hrs post-plating, the medium was aspirated and replaced with 30 μl/well of Calcium Assay Kit Component A Dye (Molecular Devices; Sunnyvale, Calif.) dissolved in 30 ml/bottle of assay buffer (2.5 mM probenecid, 20 mM Hepes, and 1× Hanks' Balanced Salt Solution (Invitrogen; Carlsbad, Calif.) (138 mM NaCl, 5.3 mM KCl, 1.3 mM CaCl₂, 0.49 mM MgCl₂, 0.41 mM MgSO₄, 0.44 mM KH₂PO₄, 0.34 mM Na₂HPO₄) pH 7.4. Plates were incubated in the dye for 1 hr at 37° C. Dilutions were made in assay buffer as 2× stocks and added to 96-well drug plates. Fluorometric imaging was performed using a FlexStation II plate reader (Molecular Devices) reading the plate at 1.5 second intervals for 1 min. After establishing a fluorescent baseline (excitation at 485 nM and emission at 525 nM, using a 515 nM cutoff), the FlexStation II automatically transferred 30 μl of 2× drug stocks from the drug plate to the cell plate at the 20 second time point with reading for another 40 seconds. Peak relative fluorescence units (RFU) were subtracted from baseline RFUs using SoftMax Pro (Molecular Devices) and values were imported into GraphPad Prism version 4.03 (GraphPad Software; San Diego, Calif.) for analysis. Ca++-flux studies are very convenient for initial screening of large numbers of compounds for agonist activity.

Compounds are resuspended in DMSO at a stock concentration of 2-15 mM and, for a typical agonist screen, are diluted in assay buffer to a final 2× concentration of 20 μM into drugs plates. Compounds are added in duplicate to cell plates using the FlexStation II as described to identify agonists, defined as having >10% of maximal 5-HT response. After this initial ˜15 min read, a concentration of 5-HT near the EC₅₀ value for each receptor (15 nM or 3 nM final for 5-HT2A or 5-HT2B and 5-HT_{2C} receptors, respectively) is then similarly added to identify antagonists, defined as reducing response to <50%. Generally, screens are performed twice with each determination being made in duplicate. Typical Z′-factor (Zhang et al., 1999) values for agonist screens ranged from 0.61-0.79 (5-HT_{2A}R), 0.42-0.81 (5-HT_{2B}R) and 0.38-0.72 (5-HT_{2C}R); these Z′-factor values are adequate for screening studies in which N=2 determinations are made and the screens replicated for a total of N=4 determinations.

Compounds identified by primary screens to be agonists are subsequently used in concentration-response experiments to verify the initial observations and obtain agonist potencies. Concentration-response studies are performed in triplicate or quadruplicate at each concentration and repeated three times. Baseline-subtracted RFU values imported into GraphPad Prism are analyzed by non-linear regression to obtain pEC₅₀ values, and normalized to maximal 5-HT response to obtain relative efficacies.

Functional studies using 5-HT2 family receptors can also be performed using PI hydrolysis as previously detailed (see Setola, V., et al. Mol Pharmacol, 63, 1223-1229, (2003), for example). In brief, cells are plated in poly-L-lysine-coated 24-well plates using DMEM containing dialyzed serum and, 24 hr prior to assay, switched to serum-free, inositol-free DMEM containing 1 microCi/ml [3H]inositol. The next day, the radioactive medium is removed and replaced with a modified Krebs-Bicarbonate buffer containing 15 mM LiC1 (to ‘trap’ inositol phosphates; (3,17)) and after a 30-min exposure to graded concentrations of agonists the reaction terminated and inositol phosphates extracted with formic acid, purified by column chromatography, and quantified by liquid scintillation spectroscopy as previously detailed (Roth, B. L., et al. Neuropharmacology, 23, 1223-1225, (1984); Roth, B. L., et al. J Pharmacol Exp Ther, 238, 480-485, (1986); Gray, J. A., et al. Mol Pharmacol, 60, 1020-1030 (2001); Gray, J. A., et al. Mol Pharmacol, 63, 961-972, (2003); Xia, Z., et al. J Biol Chem, 278, 21901-21908, (2003); Gray, J. A., et al. Biochemistry, 42, 10853-10862, (2003); Setola, V., et al. Mol Pharmacol, 63, 1223-1229, (2003)). PI hydrolysis studies can be used for precise quantification of agonist potencies and efficacies.

5-HT2C radioligand binding can be measured using [³H]mesulergine as previously detailed (Choudhary, M. S, et al. Mol Pharmacol, 42, 627-633, (1992); Rothman, R. B., et al. Circulation, 102, 2836-2841., (2000)). For radioligand binding studies, cells are seeded in 100-mm dishes in Dulbecco's Modified Eagle Medium (DMEM) containing 10% fetal bovine serum, antibiotics and the appropriate concentration of selection reagent (e.g., 5 microg/ml puromycin or 600 microg/ml G418). The next day, cells are switched to medium containing dialyzed serum (to remove serotonin) and then switched to serum-free medium for 15 hr prior to harvesting. Cells are harvested using a cell scraper, pelleted, washed twice in phosphate-buffered saline and then lysed in binding buffer (50 mM Tris-C1, 0.5 mM EDTA, 10 mM MgC12, pH 7.4). Cell membranes are then harvested by high-speed centrifugation (30,000×g for 20 min) and then frozen as tight pellets at −80 C until assay. Radioligand binding assays are performed in a total volume of 0.5 ml using the appropriate radioligand with Ki values calculated from 11-point competition binding isotherms using GraphPad Prizm (V4.0) as previously detailed (Setola, V., et al. Mol Pharmacol, 63, 1223-1229, (2003)). Typically, specific binding represents >95% of total binding.

HEK293 cells stably expressing FLAG-h5-HT₂B receptors were cultured as described above and media changed into serum-free DMEM one day prior to collection. Cells were scraped and centrifuged at 1,000×g for 10 min, media was then aspirated, and cells were resuspended in ice cold standard binding buffer (SBB: 50 mM Tris-HCl pH 7.4, 10 mM MgCl₂ and 0.1 mM EDTA). Following centrifugation at 14,000×g for 20 minutes at 4° C., the supernatant was aspirated and cell membrane fraction was stored at −80° C. for future use. 5HT2BR pellets were thawed and washed by resuspending in ice cold SBB. After removing wash buffer by centrifugation at 14,000×g for 15 minutes at 4° C., pellets were resuspended by dounce homogenization in room temperature SSB. Membranes were then incubated for 1.5 hrs at room temperature with 12 concentrations, ranging from 0.18 to 30 nM, of [N-Methyl-3H] Lysergic Acid Diethylamide, [³H]-LSD (74.2 Ci/mmol, PerkinElmer; Wellesley, Mass.) in the presence of vehicle or 10 microM SB206553 (Sigma-Aldrich) to determine total and non-specific binding, respectively. Reactions were terminated by rapid filtration onto cold 0.3% PEI presoaked filters and washed three times in 4° C. 50 mM Tris-HCl pH 6.9. Filtered material was mixed with 4 mL of EcoscintA scintillation fluid (National Diagnostic; Atlanta, Ga.) in scintillation vials and counted on a Beckman LS6500 scintillation counter (Beckman Coulter; Fullerton, Calif.). Total and nonspecific binding samples were performed in duplicate and saturation binding was repeated twice. Protein concentration was determined by Bradford protein assay (Bio-Rad; Hercules, Calif.) using BSA dilutions to generate the standard curve. K_{d} and Bₘₐₓ were determined using GraphPad Prism version 4.03 to fit total and nonspecific binding with ligand depletion.

### Synthetic Methods

### Example 1

### Preparation of trans-(±)-(2-Phenyl-cyclopropyl)-methylamine Hydrochloride

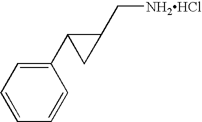

### i) trans-(±)- and cis-(±)-2-Phenyl-cyclopropanecarboxylic Acid Ethyl Ester

Under dry conditions, Cu(acac)₂ (78 mg, 0.3 mmol) was dissolved in anhydrous CH2Cl2 (20 mL). After the solution was stirred for 5 min, a few drops of phenylhydrazine were added and stirring was continued. To this solution styrene (1.15 mL, 10 mmol) was added. The mixture was stirred at 40° C. for 5 min, and a solution of ethyl diazoacetate (1.56 mL, 15 mmol) in CH₂Cl₂ (20 mL) was added via syringe pump over 5 h. After stirring for one more hour and addition of CH₂Cl₂ (50 mL), the mixture was washed successively with satd. aq. NaHCO₃ (×2) and H₂O (×2). The organic portion was dried over Na₂SO₄ and all volatiles were removed under vacuum. The isomers were separated by silica gel chromatography using a mixture of hexane/Et₂O (20:1) as an eluent to afford the title compounds as colorless oils ((±)-trans: 1.19 g and (±)-cis: 490 mg). (±)-trans: ¹H NMR (CDCl₃): δ (ppm) 7.30 (m, 2H), 7.24 (m, 1H), 7.13 (d, 2H, J=7.1 Hz), 4.20 (q, 2H, J=7.1 Hz), 2.54 (m, 1H), 1.93 (m, 1H), 1.63 (m, 1H), 1.37-1.29 (m, 4H). ¹³C NMR (CDCl₃): δ (ppm) 173.3, 140.0, 128.4 (2H), 126.4, 126.1 (2H), 60.6, 26.1, 24.1, 17.0, 14.2. (±)-cis: ¹H NMR (CDCl₃): δ (ppm) 7.29-7.21 (m, 5H), 3.90 (q, 2H, J=7.1 Hz), 2.60 (m, 1H), 2.10 (m, 1H), 1.74 (m, 1H), 1.35 (m, 1H), 0.99 (t, 3H, J=7.1 Hz). ¹³C NMR (CDCl₃): δ (ppm) 170.1, 136.5, 129.2 (2), 127.8 (2), 126.6, 60.1, 25.4, 21.7, 14.0, 11.0.

### ii) trans-(±)-2-Phenyl-cyclopropanecarboxylic Acid

A solution of trans-(±)-2-phenyl-cyclopropanecarboxylic acid ethyl ester (128 mg, 0.726 mmol) in MeOH (1 mL) was added to KOH (406 mg, 7.26 mmol) in MeOH (3 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (80 mg).

### iii) trans-(±)-2-Phenyl-cyclopropanecarboxylic Acid Amide

To a solution of trans-(±)-2-phenyl-cyclopropanecarboxylic acid (2.0 g, 12.3 mmol) in toluene (40 mL) were added dropwise several drops of dimethylformamide and thionyl chloride (13.5 mL, 185 mmol). After stirring at 80° C. for 3 h, the reaction mixture was concentrated under vacuum. The resulting residue was dissolved in toluene (10 mL) again, and the solution was added to liquid ammonia at −78° C. After stirring at −78° C. for 30 min and then at room temperature for 30 min, CH₂Cl₂ (25 ml) was added to the mixture at −78° C. and the resulting mixture was stirred at room temperature overnight. After addition of EtOAc, the mixture was washed with satd. aqueous NH4Cl (×2), dried over Na2SO4 and all volatiles were removed under vacuum. The title compound was isolated as pearl yellow powders and further purified by recrystallization from hexane/EtOAc (1.72 g). 1H NMR (DMSO-d6): δ (ppm) 7.60 (br. s, 1H), 7.29-7.10 (m, 5H), 6.91 (br. s, 1H), 2.21 (m, 1H), 1.82 (m, 1H), 1.32 (m, 1H), 1.18 (m, 1H). ¹³C NMR (DMSO-d6): δ (ppm) 173.8, 142.1, 129.2 (2), 126.8, 126.6 (2), 26.4, 24.8, 16.1.

### iv) trans-(±)-(2-Phenyl-cyclopropyl)-methylamine Hydrochloride

To a solution of trans-(±)-2-phenyl-cyclopropanecarboxylic acid amide (1.6 g, 9.93 mmol) in anhydrous THF (40 mL) was added dropwise 1 M borane/THF solution (39.7 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO4 and concentrated until the volume was reduced to about 20 mL. To the solution, was added 1 M HCl in Et₂O (20 mL, 20 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (1.54 g). ¹H NMR (methanol-d4): δ (ppm) 7.29-7.24 (m, 2H), 7.18-7.13 (m, 3H), 3.01 (d, 2H, J=7.4 Hz), 2.03 (m, 1H), 1.41 (m, 1H), 1.14-1.05 (m, 2H). ¹³C NMR (methanol-d4): δ (ppm) 141.6, 128.4 (2), 126.0, 125.9 (2), 43.9, 22.2, 19.8, 14.0. MS (ESI) 148.1 [MH+]. HRMS (ESI) calculated for C₁₀H₁₄N+ [MH+] 148.1126; found, 148.1127.

### Example 2

### Preparation of cis-(±)-(2-Phenyl-cyclopropyl)-methylamine Hydrochloride

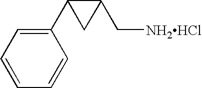

### i) cis-(±)-2-Phenyl-cyclopropanecarboxylic Acid

A solution of cis-(±)-2-phenyl-cyclopropanecarboxylic acid ethyl ester (330 mg, 1.87 mmol) in MeOH (1 mL) was added to KOH (314 mg, 5.61 mmol) in MeOH (2 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (233 mg).

### ii) cis-(±)-2-Phenyl-cyclopropanecarboxylic Acid Amide

To a solution of cis-(±)-2-phenyl-cyclopropanecarboxylic acid (230 mg, 1.42 mmol) in toluene (4 mL) were added dropwise several drops of dimethylformamide and thionyl chloride (1.55 mL, 21.3 mmol). After stirring at 80° C. for 3 h, the reaction mixture was concentrated under vacuum. The resulting residue was dissolved in toluene (2 mL) again, and the solution was added to liquid ammonia (ca. 5 mL) at −78° C. After stirring at −78° C. for 30 min and then at room temperature for 30 min, CH₂Cl₂ (25 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (128 mg).

### iii) cis-(±)-(2-Phenyl-cyclopropyl)-methylamine Hydrochloride

To a solution of cis-(±)-2-phenyl-cyclopropanecarboxylic acid amide (50 mg, 0.310 mmol) in anhydrous THF (2 mL) 1 M borane/THF solution (1.09 mL) at 0° C. was added dropwise. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about 2 mL. To the solution, was added 1 M HCl in Et₂O (1 mL, 1 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (26.8 mg). ¹H NMR (methanol-d₄): δ (ppm) 7.35-7.24 (m, 5H), 2.99-2.94 (m, 1H), 2.20 (m, 1H), 1.49 (m, 1H), 1.25-1.18 (m, 1H), 1.08 (m, 1H). ¹³C NMR (methanol-d₄): δ (ppm) 137.3, 129.1 (2), 128.5 (2), 126.8, 40.5, 21.1, 15.5, 8.3. HRMS (ESI) calculated for C₁₀H₁₄N⁺ [MH⁺] 148.1126; found, 148.1123.

### Example 3

### Preparation of trans-(±)-Methyl-(2-phenyl-cyclopropyl)-amine 7

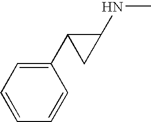

Acetic formic anhydride was generated by dropwise addition of formic acid (0.36 mL, 9.6 mmol) to acetic anhydride (0.73 mL, 7.8 mmol) maintained on ice followed at 50° C. for 2 h. The mixture was cooled to room temperature, and THF (5 mL) was added. This mixture (0.6 mL) containing acetic formic anhydride (0.3 mmol) was added to a solution of trans-(±)-2-phenyl-cyclopropylamine hydrochloride (50 mg, 0.3 mmol) in THF (1 mL) at −15° C. followed by addition of N-methylmorpholine (45 uL, 0.3 mmol). The resulting mixture was stirred at −15° C. for 30 min and at room temperature for 1 h, filtered out insoluble materials, and concentrated in vacuo. The crude residue (65 mg) was dissolved in THF (1.2 mL), and to the solution was added 1.0 M solution of borane dimethylsulfide complex in THF (0.75 mL). After the mixture was stirred at 65° C. overnight, the reaction was quenched by 10% aqueous HCl. The mixture was concentrated under reduced pressure to remove THF, and the residual aqueous solution was washed with Et₂O (×1), neutralized with 10% aqueous NaOH, and then extracted with Et₂O (×3). The combined organic layers were dried over Na₂SO₄, filtered, and concentrated. The residue was purified by preparative TLC using a mixture of EtOAc/Et₃N/MeOH (8:1:1) as a developing solvent to afford the title compound as a colorless oil (21 mg). ¹H NMR (CDCl₃): δ (ppm) 7.30-7.25 (m, 2H), 7.20-7.16 (m, 1H), 7.09-7.06 (m, 2H), 2.54 (br. s, 3H), 2.34 (m, 1H), 1.92 (m, 1H), 1.76 (br. s, 1H), 1.12-1.06 (m, 1H), 1.02-0.98 (m, 1H). ¹³C NMR (CDCl₃): δ (ppm) 142.8, 128.6 (2), 126.3 (2), 125.9, 43.7, 36.2, 25.3, 17.5. HRMS (ESI) calculated for C₁₀H₁₄N⁺ [MH⁺] 148.1126; found, 148.1124.

### Example 4

### trans-(±)-Methyl-(2-phenyl-cyclopropylmethyl)-amine 8

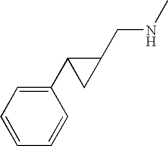

Acetic formic anhydride was generated by dropwise addition of formic acid (0.18 mL, 4.8 mmol) to acetic anhydride (0.365 mL, 3.9 mmol) maintained on ice followed at 50° C. for 2 h. The mixture was cooled to room temperature, and THF (4.5 mL) was added. This mixture (0.53 mL) containing acetic formic anhydride (0.408 mmol) was added to a solution of trans-(±)-2-phenyl-cyclopropylmethylamine hydrochloride (30 mg, 0.163 mmol) in THF (1 mL) at −15° C. followed by addition of N-methylmorpholine (17.9 uL, 0.163 mmol). The resulting mixture was stirred at −15° C. for 30 min and at room temperature for 1 h, filtered out insoluble materials, and concentrated in vacuo.

The crude residue (42 mg) was dissolved in THF (1.2 mL), and to the solution was added 1.0 M solution of borane dimethylsulfide complex in THF (0.41 mL). After the mixture was stirred at 65° C. overnight, the reaction was quenched by 10% aqueous HCl. The mixture was concentrated under reduced pressure to remove THF, and the residual aqueous solution was washed with Et₂O (×1), neutralized with 10% aqueous NaOH, and then extracted with Et₂O (×3). The combined organic layers were dried over Na₂SO₄, filtered, and concentrated. The residue was purified by preparative TLC using a mixture of EtOAc/Et₃N/MeOH (8:1:1) as a developing solvent to afford the title compound as a colorless oil (14.0 mg). ¹H NMR (CDCl₃): δ (ppm) 7.29-7.24 (m, 2H), 7.18-7.15 (m, 1H), 7.10-7.07 (m, 2H), 2.66 (br. d, 2H), 2.49 (br. s, 3H), 2.37 (br. s, 1H), 1.77 (m, 1H), 1.38-1.32 (m, 1H), 0.99-0.85 (m, 2H). ¹³C NMR (CDCl₃): δ (ppm) 143.3, 128.7, 126.2, 125.9, 56.5, 36.5, 23.3, 22.5, 15.1. HRMS (ESI) calculated for C₁₁H₁₆N⁺ [MH⁺] 162.1283; found, 162.1285.

### Example 5

### trans-(±)-Dimethyl-(2-phenyl-cyclopropyl)-amine 9

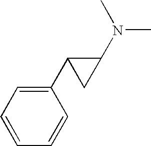

To a stirred solution of trans-(±)-2-Phenylcyclopropylamine hydrochloride (34 mg, 0.2 mmol) in acetonitrile/H₂O (1:1, v/v) (5 mL) were added N-methylmorpholine (45 uL, 0.4 mmol), 37% aqueous formaldehyde (0.16 mL, 2.0 mmol), and sodium cyanoborohydride (40 mg, 0.6 mmol). Glacial acetic acid (40 uL) was added to the mixture over 10 min and the reaction was stirred at room temperature for 30 min. To the reaction mixture was added crushed ice, and the acetonitrile was removed under vacuum. The aqueous residue was extracted with CH₂Cl₂. The combined organic layers were dried over Na₂SO₄, filtered, and concentrated. The residue was purified by preparative TLC using a mixture of EtOAc/Et₃N (95:5) as a developing solvent to afford the title compound as a colorless oil (19.7 mg). ¹H NMR (CDCl₃): δ (ppm) 7.34-7.26 (m, 2H), 7.18 (t, 1H, J=7.3 Hz), 7.09 (d, 2H, J=7.2 Hz), 2.74 (d, 2H, J=3.6 Hz), 2.42 (s, 6H), 2.00 (m, 1H), 1.83 (m, 1H), 1.13 (m, 1H), 0.99 (m, 1H). ¹³C NMR (CDCl₃): δ (ppm) 142.5, 128.6 (2), 126.5 (2), 126.0, 50.6, 45.4 (2), 25.7, 17.6. HRMS (ESI) calculated for C₁₁H₁₆N⁺ [MH⁺] 162.1283; found, 162.1279.

### Example 6

### Preparation of trans-(±)-Isopropyl-(2-phenyl-cyclopropyl)-amine 10

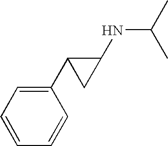

To a stirred solution of trans-(±)-2-Phenylcyclopropylamine hydrochloride (50 mg, 0.295 mmol) in MeOH (1 mL) were added acetone (21.7 uL, 0.295 mmol) and sodium cyanoborohydride (22 mg, 0.354 mmol). The mixture was stirred at 0° C. for 1 h followed at room temperature overnight. To the reaction mixture was added crushed ice, and the acetonitril was removed under vacuum. The aqueous residue was extracted with CH₂Cl₂. The combined organic layers were dried over Na₂SO₄, filtered, and concentrated. The residue was purified by preparative TLC using a mixture of EtOAc/Et₃N (95:5) as a developing solvent to afford the title compound as a colorless oil (16.7 mg). ¹H NMR (CDCl₃): δ (ppm) 7.30-7.25 (m, 2H), 7.19-7.14 (m, 1H), 7.06 (d, 2H, J=7.3 Hz), 3.01 (m, 1H), 2.31 (m, 1H), 1.90 (m, 1H), 1.87 (br. s, 1H), 1.12 (d, 6H, J=6.3 Hz), 1.07 (m, 2H). ¹³C NMR (CDCl₃): δ (ppm) 142.8, 128.6 (2), 126.1 (2), 125.8, 49.7, 40.7, 25.9, 23.7, 23.6, 17.2. HRMS (ESI) calculated for C₁₂H₁₈N⁺ [MH⁺] 176.1439; found, 176.1437.

### Example 7

### trans-(±)-Benzyl-(2-phenyl-cyclopropyl)-amine 11

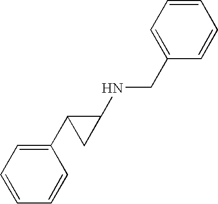

To a stirred solution of trans-(±)-2-Phenylcyclopropylamine hydrochloride (50 mg, 0.295 mmol) in MeOH (1 mL) were added benzaldehyde (27 uL, 0.266 mmol) and sodium cyanoborohydride (22 mg, 0.354 mmol). The mixture was stirred at 0° C. for 1 h followed at room temperature overnight. To the reaction mixture was added crushed ice, and the acetonitril was removed under vacuum. The aqueous residue was extracted with CH₂Cl₂. The combined organic layers were dried over Na₂SO₄, filtered, and concentrated. The residue was purified by preparative TLC using a mixture of EtOAc/Et₃N (95:5) as a developing solvent to afford the title compound as a colorless oil (15.4 mg). ¹H NMR (CDCl₃): δ (ppm) 7.40-7.25 (m, 9H), 7.18 (m, 1H), 7.03 (d, 2H, J=7.3 Hz), 3.92 (s, 2H), 2.42 (m, 1H), 2.25 (m, br. s, 1H), 1.97 (m, 1H), 1.14 (m, 1H), 1.01 (m, 1H). ¹³C NMR (CDCl₃): δ (ppm) 142.7, 140.7, 128.8 (2), 128.7 (2), 128.6 (2), 127.4, 126.3 (2), 125.9, 54.0, 41.6, 25.8, 17.5. HRMS (ESI) calculated for C₁₆H₁₈N+[MH⁺] 224.1439; found, 224.1440.

### Example 8

### trans-(±)-N-Benzyl-(2-phenyl-cyclopropylmethyl)-amine

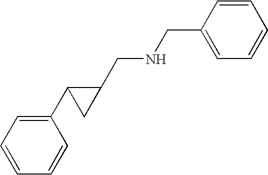

To a stirred solution of trans-(±)-2-Phenylcyclopropylmethylamine hydrochloride (30 mg, 0.163 mmol) in MeOH (1 mL) were added benzaldehyde (14.9 uL, 0.147 mmol) and sodium cyanoborohydride (12.3 mg, 0.196 mmol). The mixture was stirred at 0° C. for 1 h followed at room temperature overnight. To the reaction mixture was added crushed ice, and the acetonitril was removed under vacuum. The aqueous residue was extracted with CH₂Cl₂. The combined organic layers were dried over Na₂SO₄, filtered, and concentrated. The residue was purified by preparative TLC using a mixture of EtOAc/Et₃N (95:5) as a developing solvent to afford the title compound as a colorless oil (20.8 mg). ¹H NMR (CDCl₃): δ (ppm) 7.36-7.25 (m, 7H), 7.19-7.16 (m, 1H), 7.10-7.07 (m, 2H), 3.87 (s, 2H), 2.71 (m, 1H), 1.73 (m, 1H), 1.69 (br. s, 1H), 0.95 (m, 1H), 0.85 (m, 1H). ¹³C NMR (CDCl₃): δ (ppm) 143.4, 140.8, 128.8 (2), 128.7 (2), 128.5 (2), 127.4, 126.1 (2), 125.9, 54.1, 54.0, 23.8, 22.5, 15.3. HRMS (ESI) calculated for C₁₇H₂₀N⁺ [MH⁺] 238.1596; found, 238.1589.

### Example 9

### trans-(±)-(2-Naphthalen-2-yl-cyclopropyl)-methylamine hydrochloride

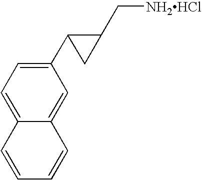

### i) 2-Naphthalen-2-yl-cyclopropanecarboxylic Acid Ethyl Ester [(±)-trans and (±)-cis]

Under dry conditions, Cu(acac)₂ (24 mg, 0.09 mmol) was dissolved in anhydrous CH₂Cl₂ (4 mL). After the solution was stirred for 5 min, a few drops of phenylhydrazine were added and stirring was continued. To this solution was added 2-vinylnaphtharene (463 mg, 3 mmol). The mixture was stirred at 40° C. for 5 min, and a solution of ethyl diazoacetate (0.467 mL, 4.5 mmol) in CH₂Cl₂ (8 mL) was added via syringe pump over 5 h. After stirring for 2 more hours and addition of CH₂Cl₂ (50 mL), the mixture was washed successively with satd. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 325 mg). (±)-trans: ¹H NMR (CDCl₃): δ (ppm) 7.80-7.78 (m, 3H), 7.60 (br. s, 1H), 7.47 (m, 2H), 7.24-7.22 (m, 1H), 4.22 (br. d, 2H, J=6.9 Hz), 2.72 (br. s, 1H), 2.04 (m, 1H), 1.71 (m, 1H), 1.46 (m, 1H), 1.33 (br. t, 3H, J=6.8 Hz). ¹³C NMR (CDCl₃): δ (ppm) 173.8, 137.9, 133.8, 132.7, 128.6, 128.0, 127.8, 126.7, 125.9, 125.2, 125.0, 61.2, 26.8, 24.6, 17.5, 14.7.

### ii) trans-(±)-2-Naphthalen-2-yl-cyclopropanecarboxylic Acid

A solution of trans-(±)-2-naphthalen-2-yl-cyclopropanecarboxylic acid ethyl ester (310 mg, 1.29 mmol) in MeOH (2 mL) was added to KOH (724 mg, 12.9 mmol) in MeOH (4.5 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (260 mg).

### iii) trans-(±)-2-Naphthalen-2-yl-cyclopropanecarboxylic Acid Amide

To a solution of trans-(±)-2-(2-naphthyl)cyclopropanecarboxylic acid (155 mg, 0.73 mmol) in toluene (3 mL) were added dropwise a few drops of dimethylformamide and thionyl chloride (0.8 mL, 11.0 mmol). After stirring at 80° C. for 3 h, the reaction mixture was concentrated under vacuum. The resulting residue was dissolved in toluene (2 mL) again, and the solution was added to liquid ammonia (ca. 5 mL) at −78° C. After stirring at −78° C. for 30 min and then at room temperature for 30 min, CH₂Cl₂ (25 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (135 mg). ¹H NMR (DMSO-d₆): δ (ppm) 7.87-7.81 (m, 3H), 7.67-7.63 (m, 2H), 7.50-7.41 (m, 2H), 7.27 (d, 1H, J=8.4 Hz), 6.94 (br. s, 1H), 2.39 (m, 1H), 1.96 (m, 1H), 1.44-1.30 (m, 2H). ¹³C NMR (DMSO-d₆): δ (ppm) 172.9, 138.8, 133.0, 131.6, 127.9, 127.5, 127.2, 126.2, 125.2, 124.6, 124.0, 25.6, 24.2, 15.3.

### iv) trans-(±)-(2-Naphthalen-2-yl-cyclopropyl)-methylamine hydrochloride

To a solution of trans-(±)-2-(2-naphthyl)cyclopropanecarboxylic amide (50 mg, 0.237 mmol) in anhydrous THF (2 mL), was added dropwise 1 M borane/THF solution (0.95 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about mL. To the solution, was added 1 M HCl in Et₂O (0.5 mL, 0.5 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (40 mg). ¹H NMR (methanol-d₄): δ (ppm) 7.81-7.77 (m, 3H), 7.63 (s, 1H), 7.48-7.38 (m, 2H), 7.28 (m, 1H), 3.06 (d, 2H, J=7.4 Hz), 2.20 (m, 1H), 1.55 (m, 1H), 1.24 (m, 1H), 1.15 (m, 1H). ¹³C NMR (methanol-d₄): δ (ppm) 139.1, 134.0, 132.7, 128.1, 127.6, 127.4, 126.2, 125.3, 124.7, 124.3, 43.9, 22.4, 19.9, 14.1. HRMS (ESI) calculated for C₁₄H₁₆N⁺ [MH⁺] 198.1283; found, 198.1290.

### Example 10

### trans-(±)-[2-(4-Bromo-phenyl)-cyclopropyl]-methylamine Hydrochloride

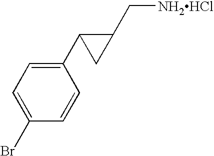

### i) 2-(4-Bromo-phenyl)-cyclopropanecarboxylic Acid Ethyl Ester ((±)-trans and (±)-cis)

Under dry conditions, Cu(acac)₂ (156 mg, 0.6 mmol) was dissolved in anhydrous CH₂Cl₂ (40 mL). After the solution was stirred for 5 min, a few drops of phenylhydrazine were added and stirring was continued. To this solution was added 4-Bromo-styrene (2.62 mL, 20 mmol). The mixture was stirred at 40° C. for 5 min, and a solution of ethyl diazoacetate (3.12 mL, 30 mmol) in CH₂Cl₂ (40 mL) was added over 5 h. After stirring for one more hour and addition of CH₂Cl₂ (100 mL), the mixture was washed successively with sat. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 955 mg). (±)-trans: ¹H NMR (CDCl₃): δ (ppm) 7.41 (d, 2H, J=8.4 Hz), 6.99 (d, 2H, J=8.4 Hz), 4.19 (q, 2H, J=7.1 Hz), 2.49 (m, 1H), 1.89 (m, 1H), 1.63 (m, 1H), 1.32-1.26 (m, 4H). ¹³C NMR (CDCl₃): δ (ppm) 173.3, 139.4, 131.7 (2), 128.1 (2), 120.3, 61.0, 25.4, 24.4, 17.2, 14.5.

### ii) trans-(±)-2-(4-Bromo-phenyl)-cyclopropanecarboxylic Acid

A solution of trans-(±)-ethyl 2-(4-Bromo-phenyl)cyclopropanecarboxylate (950 mg, 3.53 mmol) in MeOH (7.5 mL) was added to KOH (1.98 g, 35.3 mmol) in MeOH (10 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (847 mg).

### iii) trans-(±)-2-(4-Bromo-phenyl)-cyclopropanecarboxylic Acid Amide

To a solution of trans-(±)-2-(4-Bromo-phenyl)cyclopropanecarboxylic acid (400 mg, 1.66 mmol) in toluene (6 mL) were added dropwise a few drops of dimethylformamide and thionyl chloride (1.82 mL, 24.9 mmol). After stirring at 80° C. for 3 h, the reaction mixture was concentrated under vacuum. The resulting residue was dissolved in toluene (2 mL) again, and the solution was added to liquid ammonia (ca. 20 mL) at −78° C. After stirring at −78° C. for 30 min and then at room temperature for 30 min, CH₂Cl₂ (25 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (282 mg).

### iv) trans-(±)-[2-(4-Bromo-phenyl)-cyclopropyl]-methylamine hydrochloride

To a solution of trans-(±)-2-(4-Bromo-phenyl)cyclopropanecarboxylic amide (200 mg, 0.833 mmol) in anhydrous THF (3 mL), was added dropwise 1 M borane/THF solution (3.33 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about mL. To the solution, was added 1 M HCl in Et₂O (3 mL, 3 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (199 mg). MS (ESI) 225.9 [MH⁺]. HRMS (ESI) calculated for C₁₀H₁₃NBr⁺ [MH⁺] 226.0231; found, 226.0233.

### Example 11

### trans-(±)-[2-(3-Bromo-phenyl)-cyclopropyl]-methylamine Hydrochloride

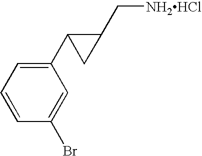

### i) 2-(3-Bromo-phenyl)-cyclopropanecarboxylic Acid Ethyl Ester ((±)-trans and (±)-cis)

Under dry conditions, Cu(acac)₂ (156 mg, 0.6 mmol) was dissolved in anhydrous CH₂Cl₂ (40 mL). After the solution was stirred for 5 min, a few drops of phenylhydrazine were added and stirring was continued. To this solution was added 3-Bromo-styrene (2.62 mL, 20 mmol). The mixture was stirred at 40° C. for 5 min, and a solution of ethyl diazoacetate (3.12 mL, 30 mmol) in CH₂Cl₂ (40 mL) was added over 5 h. After stirring for one more hour and addition of CH₂Cl₂ (100 mL), the mixture was washed successively with sat. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 2.75 g). ¹H NMR (CDCl₃): δ (ppm) 7.35 (d, 1H, J=7.8 Hz), 7.25 (s, 1H), 7.16 (t, 1H, J=7.8 Hz), 7.05 (d, 1H, J=7.7 Hz), 4.23-4.16 (m, 2H), 2.49 (m, 1H), 1.90 (m, 1H), 1.62 (m, 1H), 1.30 (m, 4H). ¹³C NMR (CDCl₃): δ (ppm) 173.4, 142.9, 130.3, 130.0, 129.7, 125.4, 123.0, 61.3, 26.0, 24.6, 17.4, 14.7.

### ii) trans-(±)-2-(3-Bromo-phenyl)-cyclopropanecarboxylic Acid

A solution of trans-(±)-ethyl 2-(3-Bromo-phenyl)cyclopropanecarboxylate (2.72 g, 10.11 mmol) in MeOH (25 mL) was added to KOH (6.67 g, 101 mmol) in MeOH (25 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (2.48 g).

### iii) trans-(±)-2-(3-Bromo-phenyl)-cyclopropanecarboxylic Acid Amide

To a solution of trans-(±)-2-(3-Bromo-phenyl)cyclopropanecarboxylic acid (2.38 g, 9.87 mmol) in toluene (30 mL) were added dropwise a few drops of dimethylformamide and thionyl chloride (10.8 mL, 148 mmol). After stirring at 80° C. for 3 h, the reaction mixture was concentrated under vacuum. The resulting residue was dissolved in toluene (10 mL) again, and the solution was added to liquid ammonia (ca. 20 mL) at −78° C. After stirring at −78° C. for 30 min and then at room temperature for 30 min, CH₂Cl₂ (25 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (1.86 g). ¹H NMR (DMSO-d₆): δ (ppm) 7.59 (br. s, 1H), 7.36 (d, 1H, J=7.9 Hz), 7.32 (s, 1H), 7.22 (t, 1H, J=7.8 Hz), 7.14 (d, 1H, J=7.7 Hz), 6.94 (br. s, 1H), 2.23 (m, 1H), 1.87 (m, 1H), 1.33 (m, 1H), 1.23 (m, 1H). ¹³C NMR (DMSO-d₆): δ (ppm) 172.7, 144.3, 130.4, 128.8, 128.4, 125.1, 121.8, 25.7, 23.4, 15.4.

### iv) trans-(±)-[2-(3-Bromo-phenyl)-cyclopropyl]-methylamine Hydrochloride

To a solution of trans-(±)-2-(3-Bromo-phenyl)cyclopropanecarboxylic amide (1.75 g, 7.29 mmol) in anhydrous THF (29 mL), was added dropwise 1 M borane/THF solution (29.2 mL) at 0 C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about 1 mL. To the solution, was added 1 M HCl in Et₂O (14.6 mL, 14.6 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (1.32 g). MS (ESI) 226.0 [MH⁺]. HRMS (ESI) calculated for C₁₀H₁₃NBr⁺ [MH⁺] 226.0231; found, 226.0235.

### Example 12

### trans-(±)-[2-(2-Bromo-phenyl)-cyclopropyl]-methylamine Hydrochloride

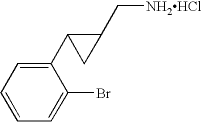

### i) 2-(2-Bromo-phenyl)-cyclopropanecarboxylic Acid Ethyl Ester ((±)-trans and (±)-cis)

Under dry conditions, Cu(acac)₂ (156 mg, 0.6 mmol) was dissolved in anhydrous CH₂Cl₂ (40 mL). After the solution was stirred for 5 min, a few drops of phenylhydrazine were added and stirring was continued. To this solution was added 2-Bromo-styrene (2.59 mL, 20 mmol). The mixture was stirred at 40° C. for 5 min, and a solution of ethyl diazoacetate (3.12 mL, 30 mmol) in CH₂Cl₂ (40 mL) was added over 5 h. After stirring for one more hour and addition of CH₂Cl₂ (100 mL), the mixture was washed successively with satd. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 2.73 g). ¹H NMR (CDCl₃): δ (ppm) 7.58 (d, 1H, J=7.9 Hz), 7.25 (t, 1H, J=7.5 Hz), 7.11 (t, 1H, J=7.6 Hz), 7.04 (d, 1H, J=7.0 Hz), 4.26-4.20 (m, 2H), 2.76-2.69 (m, 1H), 1.81 (m, 1H), 1.65 (m, 1H), 1.36-1.29 (m, 4H).

### ii) trans-(±)-2-(2-Bromo-phenyl)-cyclopropanecarboxylic Acid

A solution of trans-(±)-ethyl 2-(2-Bromo-phenyl)cyclopropanecarboxylate (2.69 g, 10 mmol) in MeOH (25 mL) was added to KOH (6.6 g, 101 mmol) in MeOH (25 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (2.28 g).

### iii) trans-(±)-2-(2-Bromo-phenyl)-cyclopropanecarboxylic Acid Amide

To a solution of trans-(±)-2-(2-Bromo-phenyl)cyclopropanecarboxylic acid (2.25 g, 9.33 mmol) in toluene (30 mL) were added dropwise a few drops of dimethylformamide and thionyl chloride (10.2 mL, 140 mmol). After stirring at 80° C. for 3 h, the reaction mixture was concentrated under vacuum. The resulting residue was dissolved in toluene (7 mL) again, and the solution was added to liquid ammonia (ca. 20 mL) at −78° C. After stirring at −78° C. for 30 min and then at room temperature for 30 min, CH₂Cl₂ (25 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (1.93 g).

### iv) trans-(±)-[2-(2-Bromo-phenyl)-cyclopropyl]-methylamine Hydrochloride

To a solution of trans-(±)-2-(2-Bromo-phenyl)-cyclopropanecarboxylic acid amide (1.93 g, 8.04 mmol) in anhydrous THF (32 mL), was added dropwise 1 M borane/THF solution (32.2 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about 1 mL. To the solution, was added 1 M HCl in Et₂O (16 mL, 16 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (1.73 g). ¹H NMR (methanol-d₄): δ (ppm) 7.58 (d, 1H, J=8.1 Hz), 7.30 (m, 1H), 7.14 (m, 2H), 3.33 (m, 1H), 2.90 (m, 1H), 2.19 (m, 1H), 1.40 (m, 1H), 1.14 (m, 2H). ¹³C NMR (methanol-d₄): δ (ppm) 140.3, 132.5, 128.2, 127.9, 17.8, 125.8, 43.8, 23.3, 18.7, 12.9. MS (ESI) 226.0 [MH⁺]. HRMS (ESI) calculated for C₁₀H₁₃NBr⁺ [MH⁺] 226.0231; found, 226.0228.

### Example 13

### trans-(±)-[2-(4-Fluoro-phenyl)-cyclopropyl]-methylamine Hydrochloride

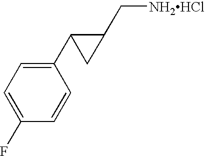

### i) 2-(4-Fluoro-phenyl)-cyclopropanecarboxylic Acid Ethyl ester ((±)-trans and (±)-cis)

Under dry conditions, Cu(acac)₂ (39.2 mg, 0.15 mmol) was dissolved in anhydrous CH₂Cl₂ (5 mL). After the solution was stirred for 5 min, a few drops of phenylhydrazine were added and stirring was continued. To this solution was added 4-fluoro-styrene (0.6 mL, 5 mmol). The mixture was stirred at 40° C. for 5 min, and a solution of ethyl diazoacetate (0.78 mL, 15 mmol) in CH₂Cl₂ (10 mL) was added via syringe pump over 5 h. After stirring for one more hour and addition of CH₂Cl₂ (50 mL), the mixture was washed successively with sat. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 494 mg and (±)-cis: 395 mg). (±)-trans isomer: ¹H NMR (CDCl₃): δ (ppm) 7.08 (m, 2H), 6.98 (m, 2H), 4.19 (q, 2H, J=7.1 Hz), 2.52 (m, 1H), 1.86 (m, 1H), 1.60 (m, 1H), 1.30 (t, 3H, J=7.1 Hz), 1.27 (m, 1H). ¹³C NMR (CDCl₃): δ (ppm) 173.5, 161.8 (¹J(C, F)=245 Hz), 135.9 (⁴J(C, F)=3.1 Hz), 128.0 (2) (³J(C, F)=7.9 Hz), 115.4 (2) (²J(C, F)=21.5 Hz), 60.9, 25.6, 24.2, 17.1, 14.4.

### ii) trans-(±)-2-(4-Fluoro-phenyl)-cyclopropanecarboxylic Acid

A solution of trans-(±)-ethyl 2-(4-fluoro-phenyl)cyclopropanecarboxylate (460 mg, 2.21 mmol) in MeOH (3 mL) was added to KOH (1.24 g, 22.1 mmol) in MeOH (8 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (313 mg).

### iii) trans-(±)-2-(4-Fluoro-phenyl)-cyclopropanecarboxylic Acid Amide

To a solution of trans-(±)-2-(4-fluoro-phenyl)cyclopropanecarboxylic acid (194 mg, 1.08 mmol) in toluene (5 mL) were added dropwise a few drops of dimethylformamide and thionyl chloride (1.18 mL, 16.2 mmol). After stirring at 80° C. for 3 h, the reaction mixture was concentrated under vacuum. The resulting residue was dissolved in toluene (3 mL) again, and the solution was added to liquid ammonia (ca. 10 mL) at −78° C. After stirring at −78° C. for 30 min and then at room temperature for 30 min, CH₂Cl₂ (15 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (150 mg). ¹H NMR (DMSO-d₆): δ (ppm) 7.58 (br. s, 1H), 7.18-7.14 (m, 2H), 7.11-7.07 (m, 2H), 6.91 (br. s, 1H), 2.23 (m, 1H), 1.79 (m, 1H), 1.31 (m, 1H), 1.16 (m, 1H). ¹³C NMR (DMSO-d₆): δ (ppm) 172.9, 160.7 (¹J(C, F)=241 Hz), 137.3 (⁴J(C, F)=2.9 Hz), 127.6 (2) (³J(C, F)=7.9 Hz), 115.0 (2) (²J(C, F)=21.3 Hz), 25.4, 23.2, 15.2.

### iv) trans-(±)-[2-(4-Fluoro-phenyl)-cyclopropyl]-methylamine Hydrochloride

To a solution of trans-(±)-2-(4-fluoro-phenyl)cyclopropanecarboxylic amide (150 mg, 0.84 mmol) in anhydrous THF (5 mL), was added dropwise 1 M borane/THF solution (3.35 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about mL. To the solution, was added 1 M HCl in Et₂O (2.5 mL, 2.5 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (100 mg). ¹H NMR (methanol-d₄): δ (ppm) 7.16 (m, 2H), 7.00 (m, 2H), 3.01 (d, 2H, J=7.4 Hz), 2.04 (m, 1H), 1.39 (m, 1H), 1.08 (m, 2H). ¹³C NMR (methanol-d₄): δ (ppm) 161.8 (¹J(C, F)=243 Hz), 137.6, 127.8 (2) (³J(C, F)=7.9 Hz), 115.0 (2) (²J(C, F)=21.7 Hz), 43.8, 21.5, 19.7, 13.9. HRMS (ESI) calculated for C₁₀H₁₃NF⁺ [MH⁺] 166.1032; found, 166.1034.

### Example 14

### trans-(±)-[2-(3-Fluoro-phenyl)-cyclopropyl]-methylamine Hydrochloride

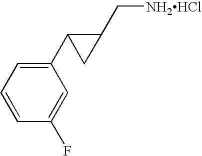

### i) 2-(3-Fluoro-phenyl)-cyclopropanecarboxylic Acid Ethyl Ester ((±)-trans and (±)-cis)

Under dry conditions, Cu(acac)₂ (39.2 mg, 0.15 mmol) was dissolved in anhydrous CH₂Cl₂ (5 mL). After the solution was stirred for 5 min, a few drops of phenylhydrazine were added and stirring was continued. To this solution was added 3-fluoro-styrene (0.6 mL, 5 mmol). The mixture was stirred at 40° C. for 5 min, and a solution of ethyl diazoacetate (0.78 mL, 15 mmol) in CH₂Cl₂ (10 mL) was added via syringe pump over 5 h. After stirring for one more hour and addition of CH₂Cl₂ (50 mL), the mixture was washed successively with sat. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 447 mg). (±)-trans isomer: ¹H NMR (CDCl₃): δ (ppm) 7.25 (m, 1H), 6.93-6.88 (m, 2H), 6.79 (d, 1H, ³J (H, F)=10.0 Hz), 4.19 (q, 2H, J=7.1 Hz), 2.52 (m, 1H), 1.91 (m, 1H), 1.63 (m, 1H), 1.33-1.28 (m, 4H). ¹³C NMR (CDCl₃): δ (ppm) 173.3, 163.2 (¹J(C, F) 246 Hz), 143.0 (³J(C, F)=7.7 Hz), 130.1 (³J (C, F)=8.5 Hz), 122.1 (⁴J(C, F)=2.8 Hz), 113.5 (²J(C, F)=21.1 Hz), 113.2 (²J(C, F)=21.9 Hz), 61.0, 25.9 (⁴J(C, F)=2.1 Hz), 24.5, 17.3, 14.4.

### ii) trans-(±)-2-(3-Fluoro-phenyl)-cyclopropanecarboxylic Acid

A solution of trans-(±)-ethyl 2-(3-fluoro-phenyl)cyclopropanecarboxylate (424 mg, 2.21 mmol) in MeOH (2 mL) was added to KOH (1.34 g, 20.4 mmol) in MeOH (8 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (356 mg).

### iii) trans-(±)-2-(3-Fluoro-phenyl)-cyclopropanecarboxylic Acid Amide

To a solution of trans-(±)-2-(3-fluoro-phenyl)cyclopropanecarboxylic acid (300 mg, 1.67 mmol) in toluene (10 mL) were added dropwise a few drops of dimethylformamide and thionyl chloride (1.83 mL, 25.1 mmol). After stirring at 80° C. for 3 h, the reaction mixture was concentrated under vacuum. The resulting residue was dissolved in toluene (3 mL) again, and the solution was added to liquid ammonia (ca. 10 mL) at −78° C. After stirring at −78° C. for 30 min and then at room temperature for 30 min, CH₂Cl₂ (15 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as pearl yellow powders and further purified by recrystallization from hexane/EtOAc (231 mg). ¹H NMR (DMSO-d₆): δ (ppm) 7.59 (br. s, 1H), 7.30 (m, 1H), 7.00-6.93 (m, 4H), 2.25 (m, 1H), 1.86 (m, 1H), 1.34 (m, 1H), 1.23 (m, 1H). ¹³C NMR (DMSO-d₆): δ (ppm) 172.7, 162.3 (¹J(C, F)=243 Hz), 144.5 (³J (C, F)=7.8 Hz), 130.1 (³J(C, F)=8.7 Hz), 122.1 (⁴J(C, F)=2.5 Hz), 112.6 (²J(C, F)=20.9 Hz), 112.4 (²J(C, F)=21.8 Hz), 25.8, 23.6, 15.5.

### iv) trans-(±)-[2-(3-Fluoro-phenyl)-cyclopropyl]-methylamine Hydrochloride

To a solution of trans-(±)-2-(3-fluoro-phenyl)cyclopropanecarboxylic amide (200 mg, 1.12 mmol) in anhydrous THF (5 mL), was added dropwise 1 M borane/THF solution (4.46 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about 1 mL. To the solution, was added 1 M HCl in Et₂O (2.24 mL, 2.24 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (157 mg). ¹H NMR (methanol-d₄): δ (ppm) 7.27 (m, 1H), 6.98 (d, 1H, J=7.7 Hz), 6.90 (m, 2H), 3.01 (m, 2H), 2.06 (m, 1H), 1.44 (m, 1H), 1.12 (m, 2H). ¹³C NMR (methanol-d₄): δ (ppm) 163.5 (¹J(C, F)=243 Hz), 144.8 (³J(C, F)=6.0 Hz), 130.1 (³J(C, F)=8.8 Hz), 122.0, 112.7 (2) (²J(C, F)=22.0 Hz), 43.7, 22.0, 20.3, 14.4. HRMS (ESI) calculated for C₁₀H₁₃NF⁺ [MH⁺] 166.1032; found, 166.1032.

### Example 15

### trans-(±)-[2-(2-Fluoro-phenyl)-cyclopropyl]-methylamine Hydrochloride

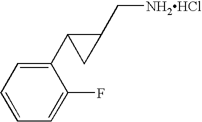

### i) 2-(2-Fluoro-phenyl)-cyclopropanecarboxylic Acid Ethyl Ester ((±)-trans and (±)-cis)

Under dry conditions, Cu(acac)₂ (39.2 mg, 0.15 mmol) was dissolved in anhydrous CH₂Cl₂ (5 mL). After the solution was stirred for 5 min, a few drops of phenylhydrazine were added and stirring was continued. To this solution was added 2-fluoro-styrene (0.6 mL, 5 mmol). The mixture was stirred at 40° C. for 5 min, and a solution of ethyl diazoacetate (0.78 mL, 15 mmol) in CH₂Cl₂ (10 mL) was added via syringe pump over 4 h. After stirring for one more hour and addition of CH₂Cl₂ (50 mL), the mixture was washed successively with sat. NaHCO₃ (×2) and H₂O (×2). The organic portion was dried over Na₂SO₄ and all volatiles were removed under vacuum. The diastereomers were separated by silica gel chromatography using a mixture of hexane/Et₂O (20:1) as an eluent to afford the title compounds as colorless oils ((±)-trans: 474 mg). (±)-trans isomer: ¹H NMR (CDCl₃): δ (ppm) 7.20 (m, 1H), 7.08-6.96 (m, 3H), 4.20 (q, 2H, J=7.1 Hz), 2.69 (m, 1H), 1.95 (m, 1H), 1.62 (m, 1H), 1.36 (m, 1H), 1.31 (t, 3H, J=7.1 Hz). ¹³C NMR (CDCl₃): δ (ppm) 173.2, 161.7 (¹J(C, F)=246 Hz), 127.8 (³J (C, F)=8.1 Hz), 127.0 (2J (C, F)=14.0 Hz), 126.9 (³J(C, F)=3.9 Hz), 124.0 (⁴J(C, F)=3.6 Hz), 115.3 (²J(C, F)=21.9 Hz), 61.0, 25.9 (⁴J(C, F)=1.2 Hz), 19.8 (³J(C, F)=4.7 Hz), 15.6, 14.2.

### ii) trans-(±)-2-(2-Fluoro-phenyl)-cyclopropanecarboxylic Acid

A solution of trans-(±)-ethyl 2-(2-fluoro-phenyl)cyclopropanecarboxylate (454 mg, 2.18 mmol) in MeOH (3 mL) was added to KOH (1.44 g, 21.8 mmol) in MeOH (8 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (349 mg).

### iii) trans-(±)-2-(2-Fluoro-phenyl)-cyclopropanecarboxylic Acid Amide

To a solution of trans-(±)-2-(2-fluoro-phenyl)cyclopropanecarboxylic acid (310 mg, 1.72 mmol) in toluene (10 mL) were added dropwise a few drops of dimethylformamide and thionyl chloride (1.88 mL, 25.8 mmol). After stirring at 80° C. for 3 h, the reaction mixture was concentrated under vacuum. The resulting residue was dissolved in toluene (4 mL) again, and the solution was added to liquid ammonia (ca. 10 mL) at −78° C. After stirring at −78° C. for 30 min and then at room temperature for 30 min, CH₂Cl₂ (15 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The title compound was isolated as a pearl yellow powder and further purified by recrystallization from hexane/EtOAc (269 mg). ¹H NMR (DMSO-d₆): δ (ppm) 7.62 (br. s, 1H), 7.23 (m, 1H), 7.17-7.05 (m, 3H), 6.95 (br. s, 1H), 2.33 (m, 1H), 1.86 (m, 1H), 1.34-1.24 (m, 2H). ¹³C NMR (DMSO-d₆): δ (ppm) 172.8, 161.0 (¹J(C, F)=243 Hz), 127.8 (³J(C, F)=4.6 Hz), 127.7 (²J (C, F)=12.9 Hz), 126.6 (³J(C, F)=4.1 Hz), 124.4 (⁴J(C, F)=3.5 Hz), 115.0 (²J(C, F) 21.7 Hz), 24.0, 17.2 (³J(C, F)=3.5 Hz), 13.7.

### iv) trans-(±)-[2-(2-Fluoro-phenyl)-cyclopropyl]-methylamine Hydrochloride

To a solution of trans-(±)-2-(2-fluoro-phenyl)cyclopropanecarboxylic amide (226 mg, 1.26 mmol) in anhydrous THF (5 mL), was added dropwise 1 M borane/THF solution (5.04 mL) at 0° C. The mixture was heated under reflux at 70° C. overnight, then was quenched by careful addition of 10% aqueous HCl. After stirring at room temperature for 1 h, the THF was removed by evaporation and the residual aqueous solution was washed with Et₂O (×2), neutralized with 10% NaOH, and then extracted with Et₂O (×4). The combined organic layers were dried over Na₂SO₄ and concentrated until the volume was reduced to about 1 mL. To the solution, was added 1 M HCl in Et₂O (2.5 mL, 2.5 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (159 mg). ¹H NMR (methanol-d₄): δ (ppm) 7.21 (m, 1H), 7.13-7.02 (m, 3H), 3.07-3.03 (m, 2H), 2.16 (m, 1H), 1.49 (m, 1H), 1.17-1.09 (m, 2H). ¹³C NMR (methanol-d₄): δ (ppm) 162.1 (¹J(C, F)=243.9 Hz), 128.3 (²J(C, F) 14.2 Hz), 127.81 (³J(C, F)=8.3 Hz), 127.2 (³J(C, F)=3.9 Hz), 124.4 (⁴J(C, F) 3.5 Hz), 115.0 (²J(C, F)=22.1 Hz), 43.8, 18.3, 15.7, 12.7. HRMS (ESI) calculated for C₁₀H₁₃NF⁺ [MH⁺] 166.1032; found, 166.1029.

### Example 16

### trans-(±)-[2-(4′-Fluoro-biphenyl-4-yl)-cyclopropyl]-methylamine Hydrochloride

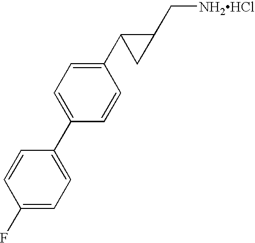

### i) trans-(±)-[2-(4-Bromo-phenyl)-cyclopropylmethyl]-carbamic Acid Tert-Butyl Ester

To a solution of trans-(±)-2-(4-Bromo-phenyl)-cyclopropylmethylamine hydrochloride (350 mg, 1.33 mmol) and Boc₂O (378 mg, 1.73 mmol) in Et₂O (16 mL) was added 10% aqueous NaOH (3.2 mL, 7.98 mmol) at 0° C. The mixture was stirred at 0° C. for 30 min and then at room temperature for 7 h. To the resulting mixture were added crushed ice and Et₂O. The organic layer was further washed with water (×1), dried over Na₂SO₄ and concentrated under vacuum. The residue was purified by silica gel chromatography using a mixture of hexane/EtOAc (10:1) as an eluent to afford the title compounds as colorless oils (356 mg). ¹H NMR (CDCl₃): δ (ppm) 7.38 (d, 2H, J=8.3 Hz), 6.93 (d, 2H, J=8.3 Hz), 4.69 (br. s, 1H), 3.18-3.11 (m, 2H), 1.79-1.76 (m, 2H), 1.47 (s, 9H), 1.28 (m, 2H), 0.94-0.90 (m, 2H). ¹³C NMR (CDCl₃): δ (ppm) 156.0, 142.0, 131.7 (2), 128.0 (2), 127.0, 79.9, 45.0, 28.8 (3), 23.7, 21.9, 14.8.

### ii) trans-(±)-[2-(4′-Fluoro-biphenyl-4-yl)-cyclopropylmethyl]-carbamic Acid Tert-Butyl Ester

trans-(±)-[2-(4-bromo-phenyl)-cyclopropylmethyl]-carbamic acid tert-butyl ester (34 mg, 0.104 mmol), Pd(PPh₃)₄ (12.0 mg, 0.01 mmol) and 2 M aqueous K₂CO₃ (0.13 mL, 0.26 mmol) were dissolved in dimethoxy ethane (DME) (3 mL), and the mixture was degassed for 5 min and stirred for 10 min at room temperature. To the mixture was added 4-fluorophenylboronic acid (36.5 mg, 0.261 mmol), degassed again for 1 min, and then stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (22 mg). ¹H NMR (CDCl₃): δ (ppm) 7.55-7.51 (m, 2H), 7.45 (d, 2H, J=8.1 Hz), 7.16-7.10 (m, 4H), 4.77 (br. s, 1H), 3.25-3.14 (m, 2H), 1.86 (m, 1H), 1.49 (s, 9H), 1.36 (m, 1H), 1.01-0.95 (m, 2H). ¹³C NMR (CDCl₃): δ (ppm) 164.4, 156.5, 142.2, 138.1, 128.9 (2), 127.4 (2), 126.7 (2), 116.6, 116.1, 79.9, 45.2, 28.9 (3), 23.7, 22.1, 14.9.

### iii) trans-(±)-[2-(4′-Fluoro-biphenyl-4-yl)-cyclopropyl]-methylamine Hydrochloride

To a solution of trans-(±)-[2-(4′-Fluoro-biphenyl-4-yl)-cyclopropylmethyl]-carbamic acid tert-butyl ester (20 mg, 0.058 mmol) in CH₂Cl₂ (1 mL) was added TFA (0.1 mL) at 0° C. After standing at 0° C. for 30 min and at room temperature for 1 h, the mixture was concentrated. The residue was dissolved in Et₂O (0.1 mL), and 1 M HCl in Et₂O (0.176 mL, 0.176 mmol) was added to the solution. The mixture was stand at 0° C. for 30 min, and concentrated. The resulting white solid was purified by recrystallization from ethanol/Et₂O to afford the title compound as a white solid (14.0 mg). ¹H NMR (methanol-d₄): δ 7.70 (m, 2H), 7.51 (d, 2H, J=8.1 Hz), 7.22 (d, 2H, J=8.1 Hz), 7.16 (m, 2H), 3.03 (d, 2H, J=7.4 Hz), 2.06 (m, 1H), 1.49 (m, 1H), 1.13 (m, 2H) (ppm). ¹³C NMR (methanol-d₄): [symbol] (ppm) 157.0, 140.9, 138.2, 137.5, 128.6, 128.5, 126.9 (2), 126.5 (2), 115.6, 115.3, 43.9, 21.9, 20.0, 14.1. HRMS (ESI) calculated for C₁₆H₁₇NF⁺ [MH⁺] 242.1345; found, 242.1344.

### Example 17

### trans-(±)-[2-(4′-Trifluoromethyl-biphenyl-4-yl)-cyclopropyl]-methylamine Hydrochloride

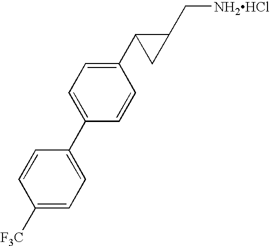

### i) trans-(±)-[2-(4′-Trifluoromethyl-biphenyl-4-yl)-cyclopropylmethyl]-carbamic Acid Tert-Butyl Ester

trans-(±)-[2-(4-bromo-phenyl)-cyclopropylmethyl]-carbamic acid tert-butyl ester (30 mg, 0.092 mmol), Pd(PPh₃)₄ (10.4 mg, 0.009 mmol) and 2 M aqueous K₂CO₃ (0.115 mL, 0.23 mmol) were dissolved in dimethoxy ethane (DME) (4 mL), and the mixture was degassed for 3 min and stirred for 10 min at room temperature. To the mixture was added 4-trifluoromethylphenylboronic acid (43.7 mg, 0.23 mmol), degassed again for 1 min, and then stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (22.2 mg). ¹H NMR (CDCl₃): δ (ppm) 7.69 (s, 4H), 7.52 (d, 2H, J=8.0 Hz), 7.17 (d, 2H, J=8.1 Hz), 4.76 (br. s, 1H), 3.25-3.15 (m, 2H), 1.88 (m, 1H), 1.49 (s, 9H), 1.37 (m, 1H), 1.00 (m, 2H). ¹³C NMR (CDCl₃): δ (ppm) 156.4, 144.8 143.3, 137.5, 130.0, 127.6 (2), 127.5 (2), 126.8 (2), 126.1, 126.0, 115.9, 79.7, 45.1, 28.8 (3), 23.8, 22.1, 15.0.

### ii) trans-(±)-[2-(4′-Trifluoromethyl-biphenyl-4-yl)-cyclopropyl]-methylamine Hydrochloride

To a solution of trans-(±)-[2-(4′-trifluoromethyl-biphenyl-4-yl)-cyclopropylmethyl]-carbamic acid tert-butyl ester (20 mg, 0.051 mmol) in CH₂Cl₂ (1 mL) was added TFA (0.1 mL) at 0° C. After standing at 0° C. for 30 min and at room temperature for 1 h, the mixture was concentrated. The residue was dissolved in Et₂O (0.1 mL), and 1 M HCl in Et₂O (0.153 mL, 0.153 mmol) was added, stand at 0° C. for 30 min, and concentrated. The resulting white solid was purified by recrystallization from ethanol/Et₂O to afford the title compound as a white solid (16.2 mg). ¹H NMR (methanol-d₄): δ (ppm) 7.37 (s, 5H), 5.14 (s, 2H), 4.00 (br. s, 2H), 2.27 (m, 1H). ¹³C NMR (DMSO-d₆): δ (ppm) 144.9, 142.1, 137.5, 128.8, 127.5 (2), 127.2 (2), 126.7 (2), 125.8, 125.7, 43.8, 21.9, 20.2, 14.3. MS (ESI) 292.1 [MH⁺]. HRMS (ESI) calculated for C₁₇H₁₇NF₃⁺ [MH⁺] 292.1313; found, 292.1318.

### Example 18

### trans-(±)-[2-(4-Pyridin-4-yl-phenyl)-cyclopropyl]-methylamine Hydrochloride

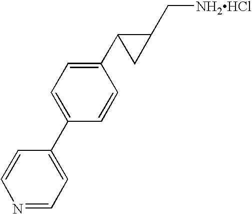

### i) trans-(±)-[2-(4-Pyridin-4-yl-phenyl)-cyclopropylmethyl]-carbamic Acid Tert-Butyl Ester

trans-(±)-[2-(4-bromo-phenyl)-cyclopropylmethyl]-carbamic acid tert-butyl ester (30 mg, 0.092 mmol), Pd(PPh₃)₄ (10.4 mg, 0.009 mmol) and 2 M aqueous K₂CO₃ (0.115 mL, 0.23 mmol) were dissolved in dimethoxy ethane (DME) (4 mL), and the mixture was degassed for 5 min and stirred for 10 min at room temperature. To the mixture was added 4-pyridylboronic acid (28.3 mg, 0.23 mmol), the mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (26.9 mg). ¹H NMR (CDCl₃): δ (ppm) 8.64 (d, 2H, J=5.5 Hz), 7.55 (d, 2H, J=8.0 Hz), 7.49 (d, 2H, J=5.4 Hz), 7.17 (d, 2H, J=8.0 Hz), 4.77 (br. s, 1H), 3.23-3.17 (m, 2H), 1.89 (m, 1H), 1.47 (s, 9H), 1.37 (m, 1H), 1.00 (m, 2H). ¹³C NMR (CDCl₃): δ (ppm) 156.3, 150.6 (2), 148.4, 144.4, 135.8, 127.3 (2), 126.9 (2), 121.7 (2), 79.7, 45.0, 28.8 (3), 24.0, 22.1, 15.1.

### ii) trans-(±)-[2-(4-Pyridin-4-yl-phenyl)-cyclopropyl]-methylamine Hydrochloride

To a solution of trans-(±)-[2-(4-Pyridin-4-yl-phenyl)-cyclopropylmethyl]-carbamic acid tert-butyl ester (12 mg, 0.037 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.148 mL, 0.148 mmol) was added, stand at 0° C. for 30 min, and concentrated. The resulting white solid was purified by recrystallization from ethanol/Et₂O to afford the title compound as a white solid (11.0 mg). ¹H NMR (methanol-d₄): δ 8.84 (d, 2H, J=6.6 Hz), 8.40 (d, 2H, J=6.6 Hz), 7.96 (d, 2H, J=8.3 Hz), 7.43 (d, 2H, J=8.3 Hz), 3.07 (d, 2H, J=7.3 Hz), 2.18 (m, 1H), 1.58 (m, 1H), 1.25 (m, 2H) (ppm). ¹³C NMR (DMSO-d₆): δ (ppm) 158.0, 147.3, 141.8, 132.2, 128.3 (2), 127.4 (2), 124.0 (2), 43.6, 22.2, 20.9, 15.0. HRMS (ESI) calculated for C₁₅H₁₇N₂⁺ [MH⁺] 225.1392; found, 225.1390.

### Example 19

### trans-(±)-[2-(4-Furan-2-yl-phenyl)-cyclopropyl]-methylamine Hydrochloride

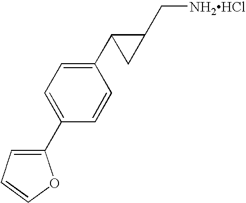

### i) trans-(±)-[2-(4-Furan-2-yl-phenyl)-cyclopropylmethyl]-carbamic Acid Tert-Butyl Ester

trans-(±)-[2-(4-bromo-phenyl)-cyclopropylmethyl]-carbamic acid tert-butyl ester (30 mg, 0.092 mmol), Pd(PPh₃)₄ (10.4 mg, 0.009 mmol) and 2 M aqueous K₂CO₃ (0.115 mL, 0.23 mmol) were dissolved in dimethylformamide (DMF) (4 mL), and the mixture was degassed for 3 min and stirred for 10 min at room temperature. To the mixture was added 2-furanboronic acid (25.7 mg, 0.23 mmol), degassed again for 1 min, and then stirred at 100° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (2:1) as a developing solvent to afford the title compound as a colorless oil (28.5 mg). ¹H NMR (DMSO-d₆): δ (ppm) 7.60 (br. s, 1H), 7.29-7.10 (m, 5H), 6.91 (br. s, 1H), 2.21 (m, 1H), 1.82 (m, 1H), 1.32 (m, 1H), 1.18 (m, 1H). ¹³C NMR (DMSO-d₆): δ (ppm) 156.3, 154.4, 142.2, 142.1, 128.9, 126.4 (2), 124.2 (2), 112.0, 104.7, 79.7, 45.1, 28.8 (3), 23.6, 22.2, 14.8.

### ii) trans-(±)-[2-(4-Furan-2-yl-phenyl)-cyclopropyl]-methylamine Hydrochloride

To trans-(±)-[2-(4-Furan-2-yl-phenyl)-cyclopropylmethyl]-carbamic acid tert-butyl ester (28 mg, 0.09 mmol) was added 4 N HCl in dioxane (0.5 mL, 2 mmol). After standing at 0° C. for 30 min and at room temperature for 2 h, the mixture was concentrated. The resulting white solid was purified by recrystallization from ethanol/Et₂O to afford the title compound as a white solid (18.4 mg). MS (ESI) 214.1 [MH+]. HRMS (ESI) calculated for C₁₄H₁₆NO⁺ [MH⁺] 214.1232; found, 214.1228.

### Example 20

### trans-(±)-[2-(4′-Chloro-biphenyl-4-yl)-cyclopropyl]-methylamine Hydrochloride

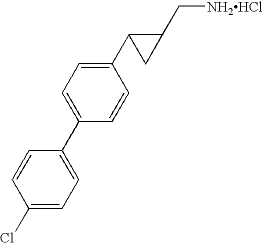

### i) trans-(±)-[2-(4′-Chloro-biphenyl-4-yl)-cyclopropylmethyl]-carbamic Acid Tert-Butyl Ester

trans-(±)-[2-(4-bromo-phenyl)-cyclopropylmethyl]-carbamic acid tert-butyl ester (30 mg, 0.092 mmol), Pd(PPh₃)₄ (10.4 mg, 0.009 mmol) and 2 M aqueous K₂CO₃ (0.115 mL, 0.23 mmol) were dissolved in dimethoxy ethane (DME) (4 mL), and the mixture was degassed for 5 min and stirred for 10 min at room temperature. To the mixture was added 4-chloro-phenylboronic acid (28.3 mg, 0.23 mmol). The mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (23.7 mg). ¹H NMR (CDCl₃): [symbol] (ppm) 7.52-7.39 (m, 6H), 7.14 (d, 2H, J=8.2 Hz), 4.75 (br. s, 1H), 3.25-3.12 (m, 2H), 1.87 (m, 1H), 1.48 (s, 9H), 1.36 (m, 1H), 0.99 (m, 2H). ¹³C NMR (CDCl₃): δ (ppm) 156.3, 142.6, 139.8, 137.8, 133.5, 129.3 (2), 128.5 (2), 127.3 (2), 126.7 (2), 79.8, 45.1, 28.8 (3), 23.7, 22.0, 14.9.

### ii) trans-(±)-[2-(4′-Chloro-biphenyl-4-yl)-cyclopropyl]-methylamine Hydrochloride

To trans-(±)-[2-(4′-Chloro-biphenyl-4-yl)-cyclopropylmethyl]-carbamic acid tert-butyl ester (20 mg, 0.056 mmol) was added 4 N HCl in dioxane (0.3 mL, 1.2 mmol). After standing at 0° C. for 30 min and at room temperature for 2 h, the mixture was concentrated. The resulting white solid was purified by recrystallization from ethanol/Et₂O to afford the title compound as a white solid (13.1 mg). ¹H NMR (methanol-d₄): [symbol] (ppm) 7.56 (m, 4H), 7.43 (d, 2H, J=8.5 Hz), 7.23 (d, 2H, J=8.1 Hz), 3.03 (d, 2H, J=7.4 Hz), 2.07 (m, 1H), 1.46 (m, 1H), 1.14 (m, 2H). ¹³C NMR (methanol-d₄): δ (ppm) 128.9 (3), 128.2 (3), 126.9 (3), 126.6 (3), 43.8, 21.9, 20.0, 14.2. MS (ESI) 258.0 [MH+].

### Example 21

### trans-(±)-[2-(4′-Chloro-biphenyl-3-yl)-cyclopropyl]-methylamine Hydrochloride

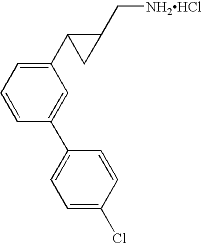

### i) trans-(±)-[2-(3-Bromo-phenyl)-cyclopropylmethyl]-carbamic Acid Tert-Butyl Ester

To a solution of trans-(±)-2-(3-Bromo-phenyl)-cyclopropylmethylamine hydrochloride (1.29 g, 4.91 mmol) and Boc₂O (1.39 g, 6.39 mmol) in Et₂O (59 mL) was added 10% aqueous NaOH (11.8 mL, 29.5 mmol) at 0° C. The mixture was stirred at 0° C. for 30 min and then at room temperature for 7 h. To the resulting mixture were added crushed ice and Et₂O. The organic layer was further washed with water (×1), dried over Na₂SO₄ and concentrated under vacuum. The residue was purified by silica gel chromatography using a mixture of hexane/EtOAc (10:1) as an eluent to afford the title compounds as colorless oils (1.67 g).

### ii) trans-(±)-[2-(4′-Chloro-biphenyl-3-yl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 4-chloro-phenylboronic acid (36.0 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (27 mg). ¹H NMR (CDCl₃): δ (ppm) 7.51 (d, 2H, J=8.6 Hz), 7.41 (d, 2H, J=8.6 Hz), 7.36-7.34 (m, 2H), 7.25 (s, 1H), 7.05 (m, 1H), 4.74 (br. s, 1H), 3.23-3.12 (m, 2H), 1.89 (m, 1H), 1.48 (s, 9H), 1.41 (m, 1H), 1.02-0.95 (m, 2H). ¹³C NMR (CDCl₃): δ (ppm) 156.4, 143.6, 140.5, 140.1, 133.8, 129.3 (3), 128.8 (2), 125.3, 125.1, 124.8, 45.1, 28.8 (3), 23.6, 22.4, 14.8.

### iii) trans-(±)-[2-(4′-Chloro-biphenyl-3-yl)-cyclopropyl]-methylamine Hydrochloride

To a solution of trans-(±)-[2-(4′-Chloro-biphenyl-3-yl)-cyclopropylmethyl]-carbamic acid tert-butyl ester (26 mg) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.4 mL) was added to the solution. The mixture was stand at 0° C. for 30 min, and concentrated. The resulting white solid was purified by recrystallization from ethanol/Et₂O to afford the title compound as a white solid (17 mg). ¹H NMR (methanol-d₄): [symbol] (ppm) 7.58 (d, 2H, J=8.4 Hz), 7.43 (d, 2H, J=8.1 Hz), 7.40-7.33 (m, 3H), 7.12 (d, 2H, J=7.5 Hz), 3.02 (m, 2H), 2.09 (m, 2H), 1.46 (m, 1H), 1.18 (m, 1H), 1.10 (m, 1H). ¹³C NMR (methanol-d₄): δ (ppm) 143.6, 141.5, 141.2, 134.6, 130.3, 130.1 (2), 129.7 (2), 126.3, 126.0, 125.9, 45.0, 23.4, 21.1, 15.2.

### Example 22

### trans-(±)-[2-(4′-Trifluoromethyl-biphenyl-3-yl)-cyclopropyl]-methylamine Hydrochloride

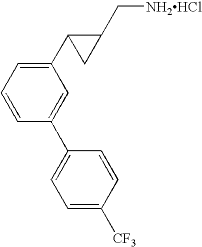

### i) trans-(±)-[2-(4′-Trifluoromethyl-biphenyl-3-yl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 4-trifluoromethyl-phenylboronic acid (43.7 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (24 mg). ¹H NMR (CDCl₃): [symbol] (ppm) 7.69 (m, 4H), 7.39-7.35 (m, 2H), 7.30 (m, 1H), 7.10 (d, 1H, J=6.5 Hz), 4.75 (br. s, 1H), 3.23-3.14 (m, 2H), 1.91 (m, 1H), 1.48 (s, 9H), 1.40 (m, 1H), 1.01 (m, 1H). ¹³C NMR (CDCl₃): δ (ppm) 156.3, 145.2, 143.8, 140.3, 129.5, 129.4, 127.9 (2), 126.5, 126.1, 126.0, 125.9, 125.4, 125.1, 79.7, 45.1, 28.8 (3), 23.7, 22.4, 14.8.

### ii) trans-(±)-[2-(4′-Trifluoromethyl-biphenyl-3-yl)-cyclopropyl]-methylamine Hydrochloride

To a solution of trans-(±)-[2-(4′-trifluoromethyl-biphenyl-3-yl)-cyclopropylmethyl]-carbamic acid tert-butyl ester (22 mg) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.3 mL) was added to the solution. The mixture was stand at 0° C. for 30 min, and concentrated. The resulting white solid was purified by recrystallization from ethanol/Et₂O to afford the title compound as a white solid (15 mg). ¹H NMR (methanol-d₄): [symbol] (ppm) 7.81 (d, 2H, J=8.2 Hz), 7.75 (d, 2H, J=8.4 Hz), 7.51-7.47 (m, 2H), 7.41 (t, 1H, J=7.7 Hz), 7.19 (d, 1H, J=7.7 Hz), 3.07-3.00 (m, 2H), 2.16-2.11 (m, 1H), 1.52-1.48 (m, 1H), 1.24-1.20 (m, 1H), 1.16-1.11 (m, 1H). ¹³C NMR (methanol-d₄): δ (ppm) 144.4, 141.8, 139.2, 128.4, 126.8 (2), 125.0, 124.9, 124.9, 124.4, 124.3, 43.1, 21.5, 19.2, 13.3.

### Example 23

### trans-(±)-[2-(4′-Fluoro-biphenyl-3-yl)-cyclopropyl]-methylamine Hydrochloride

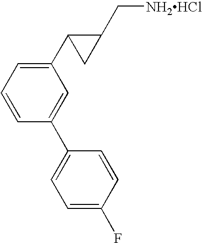

### i) trans-(±)-[2-(4′-Fluoro-biphenyl-3-yl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 4-fluoro-phenylboronic acid (32.2 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 0.1 N HCl/EtOAc (15 mL/15 mL). After partition, the organic layer was washed with water, filtered, and concentrated. The residue was purified by preparative TLC using a mixture of hexane/EtOAc (4:1) as a developing solvent to afford the title compound as a colorless oil (36 mg). ¹H NMR (CDCl₃): δ (ppm) 7.56-7.52 (m, 2H), 7.34 (br. d, 2H, J=4.5 Hz), 7.25 (br. s, 1H), 7.14 (t, 2H, J=8.7 Hz), 7.05-7.03 (m, 1H), 4.78 (br. s, 1H), 3.24-3.15 (m, 2H), 1.89 (m, 1H), 1.48 (s, 9H), 1.39 (m, 1H), 0.99 (m, 2H). ¹³C NMR (CDCl₃): δ (ppm) 162.9 (¹J(C, F)=246.4 Hz), 156.4, 143.6, 140.8, 137.7 (⁴J(C, F)=3.2 Hz), 129.2 (2) (³J(C, F)=5.2 Hz), 129.0, 125.1, 125.0, 124.9, 116.0 (²J(C, F)=21.4 Hz), 79.8, 45.1, 28.8 (3), 23.6, 22.4, 14.8.

### ii) trans-(±)-[2-(4′-Fluoro-biphenyl-3-yl)-cyclopropyl]-methylamine Hydrochloride

To a solution of trans-(±)-[2-(4′-fluoro-biphenyl-3-yl)-cyclopropylmethyl]-carbamic acid tert-butyl ester (35 mg, 0.103 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.308 mL, 0.308 mmol) was added to the solution. The mixture was stand at 0° C. for 30 min, and concentrated. The resulting white solid was purified by recrystallization from ethanol/Et₂O to afford the title compound as a white solid (18 mg). ¹H NMR (methanol-d₄): (CDCl₃): δ (ppm) 7.63-7.60 (m, 1H), 7.40-7.33 (m, 3H), 7.17 (t, 1H, J=8.8 Hz), 7.11 (d, 1H, J=7.5 Hz), 3.09-3.00 (m, 2H), 2.14-2.09 (m, 1H), 1.52-1.47 (m, 1H), 1.21-1.16 (m, 1H), 1.14-1.09 (m, 1H). ¹³C NMR (methanol-d₄): δ (ppm) 164.0 (¹J(C, F)=245 Hz), 143.5, 141.7, 138.8 (⁴J(C, F)=3.2 Hz), 130.2, 130.0 (2) (³J(C, F)=8.1 Hz), 126.0, 125.9, 125.8, 116.6 (2) (²J(C, F)=21.7 Hz), 45.0, 23.4, 21.1, 15.2.

### Example 24

### trans-(±)-3′-(2-Aminomethyl-cyclopropyl)-biphenyl-4-carbonitrile Hydrochloride

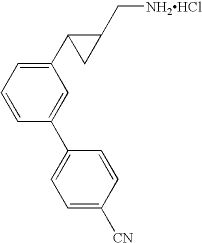

### i) [2-(4′-Cyano-biphenyl-3-yl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 4-cyano-phenylboronic acid (33.8 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 5% HCl/EtOAc (15 mL/15 mL). After partition, the organic layer was washed with water, filtered, and concentrated. The residue was purified by preparative TLC using a mixture of hexane/EtOAc (4:1) as a developing solvent to afford the title compound as a colorless oil (30 mg). ¹H NMR (CDCl₃): δ (ppm) 7.72 (d, 2H, J=8.4 Hz), 7.67 (d, 2H, J=8.3 Hz), 7.37 (d, 2H, J=4.9 Hz), 7.28 (s, 1H), 7.10 (m, 1H), 4.77 (br. s, 1H), 3.21 (m, 2H), 1.91 (m, 1H), 1.46 (s, 9H), 1.40 (m, 1H), 1.01-0.96 (m, 2H). ¹³C NMR (CDCl₃): δ (ppm) 156.3, 146.1, 144.0, 139.7, 132.9, 129.5, 128.2 (2), 126.3, 125.4, 125.0, 119.3, 111.3, 79.7, 45.0, 28.8 (3H), 23.8, 22.3, 14.9.

### ii) trans-(±)-3′-(2-Aminomethyl-cyclopropyl)-biphenyl-4-carbonitrile Hydrochloride

To a solution of trans-(±)-[2-(4′-Cyano-biphenyl-3-yl)-cyclopropylmethyl]-carbamic acid tert-butyl ester (29 mg, 0.083 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.250 mL, 0.250 mmol) was added to the solution. The mixture was stand at 0° C. for 30 min, and concentrated. The resulting white solid was purified by recrystallization from ethanol/Et₂O to afford the title compound as a white solid (20 mg). ¹H NMR (methanol-d₄): [symbol] (ppm) 7.81 (s, 4H), 7.49 (m, 1H), 7.48 (s, 1H), 7.41 (t, 1H, J=7.6 Hz), 7.21 (d, 1H, J=7.6 Hz), 3.10-3.01 (m, 2H), 2.17-2.12 (m, 1H), 1.55-1.47 (m, 1H), 1.24-1.19 (m, 1H), 1.16-1.11 (m, 1H). ¹³C NMR (methanol-d₄): δ (ppm) 147.2, 144.0, 140.7, 133.9 (2), 130.5, 129.1 (2), 127.3, 126.3, 126.2, 119.9, 112.0, 45.0, 23.4, 21.2, 15.3.

### Example 25

### trans-(±)-[2-(3′-Chloro-biphenyl-3-yl)-cyclopropyl]-methylamine Hydrochloride

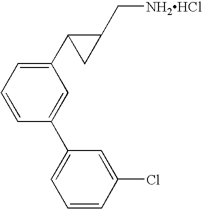

### i) trans-(±)-[2-(3′-Chloro-biphenyl-3-yl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 3-chloro-phenylboronic acid (36.0 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (35 mg). ¹H NMR (CDCl₃): δ (ppm) 7.57 (s, 1H), 7.48-7.45 (m, 1H), 7.41-7.32 (m, 4H), 7.26 (s, 1H), 7.08 (m, 1H), 4.82 (br. s, 1H), 3.25-3.19 (m, 2H), 1.90 (m, 1H), 1.50 (s, 9H), 1.40 (m, 1H), 1.05-0.96 (m, 2H). ¹³C NMR (CDCl₃): δ (ppm) 156.6, 143.5, 140.3, 135.0, 130.4, 129.3, 127.7 (2), 125.8, 125.6, 125.2, 125.0, 120.6, 80.0, 45.1, 28.9 (3), 23.6, 22.4, 14.8.

### ii) trans-(±)-[2-(3′-Chloro-biphenyl-3-yl)-cyclopropyl]-methylamine Hydrochloride

To a solution of trans-(±)-[2-(3′-chloro-biphenyl-3-yl)-cyclopropylmethyl]-carbamic acid tert-butyl ester (34 mg, 0.098 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.293 mL, 0.293 mmol) was added to the solution. The mixture was stand at 0° C. for 30 min, and concentrated. The resulting white solid was purified by recrystallization from ethanol/Et₂O to afford the title compound as a white solid (22 mg). ¹H NMR (methanol-d₄): [symbol] (ppm) 7.61 (s, 1H), 7.54 (d, 1H, J=7.7 Hz), 7.45-7.34 (m, 5H), 7.15 (d, 1H, J=7.4 Hz), 3.07-3.01 (m, 2H), 2.12 (m, 1H), 1.49 (m, 1H), 1.20 (m, 1H), 1.12 (m, 1H). ¹³C NMR (methanol-d₄): δ (ppm) 144.6, 143.7, 141.3, 135.9, 131.5, 130.3, 128.5, 128.1, 126.6, 126.6, 126.2, 126.0, 45.0, 23.4, 21.1, 15.2.

### Example 26

### trans-(±)-[2-(2′-Chloro-biphenyl-3-yl)-cyclopropyl]-methylamine Hydrochloride

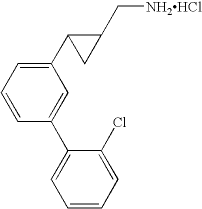

### i) trans-(±)-[2-(2′-Chloro-biphenyl-3-yl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 2-chloro-phenylboronic acid (36.0 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 0.1 N HCl/EtOAc (15 mL/15 mL). After partition, the organic layer was washed with water, filtered, and concentrated. The residue was purified by preparative TLC using a mixture of hexane/EtOAc (4:1) as a developing solvent to afford the title compound as a colorless oil (37 mg). ¹H NMR (CDCl₃): δ (ppm) 7.49 (m, 1H), 7.36-7.25 (m, 5H), 7.13-7.09 (m, 2H), 4.77 (br. s, 1H), 3.24-3.20 (m, 1H), 3.18-3.13 (m, 1H), 1.88 (m, 1H), 1.48 (s, 9H), 1.37 (m, 1H), 1.01-0.94 (m, 2H). ¹³C NMR (CDCl₃): δ (ppm) 155.9, 142.4, 140.5, 139.4, 132.5, 131.3, 129.9, 128.5, 128.0, 126.9, 126.8, 126.7, 125.1, 79.3, 44.7, 28.4 (3), 23.2, 21.9, 14.4.

### ii) trans-(±)-[2-(2′-Chloro-biphenyl-3-yl)-cyclopropyl]-methylamine Hydrochloride

To a solution of trans-(±)-[2-(2′-chloro-biphenyl-3-yl)-cyclopropylmethyl]-carbamic acid tert-butyl ester (36 mg, 0.103 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.310 mL, 0.310 mmol) was added to the solution. The mixture was stand at 0° C. for 30 min, and concentrated. The resulting white solid was purified by recrystallization from ethanol/Et₂O to afford the title compound as a white solid (25 mg). ¹H NMR (methanol-d₄): δ (ppm) 7.49 (d, 1H, J=7.8 Hz), 7.37-7.32 (m, 4H), 7.22 (d, 1H, J=7.8 Hz), 7.19-7.17 (m, 2H), 3.03 (d, 2H, J=7.4 Hz), 2.10 (m, 1H), 1.48 (m, 1H), 1.19-1.09 (m, 2H). ¹³C NMR (methanol-d₄): δ (ppm) 142.8, 142.0, 141.1, 133.5, 132.6, 131.1, 130.0, 129.4, 128.3, 128.2, 128.2, 126.4, 45.0, 23.3, 21.1, 15.3.

### Example 27

### trans-(±)-[2-(3-Benzofuran-2-yl-phenyl)-cyclopropyl]-methylamine Hydrochloride

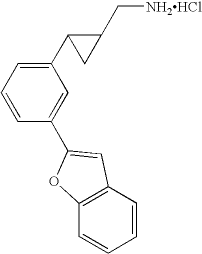

### i) [2-(3-Benzofuran-2-yl-phenyl)-cyclopropylmethyl]-carbamic Acid Tert-Butyl Ester

trans-(±)-[2-(3-bromo-phenyl)-cyclopropylmethyl]-carbamic acid tert-butyl ester (30 mg, 0.092 mmol), Pd(PPh₃)₄ (10.4 mg, 0.009 mmol), and 2-benzofuranboronic acid (37.3 mg, 0.23 mmol) were dissolved in DMF (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 100° C. overnight. The resulting mixture was cooled to ambient temperature and poured into a mixture of 0.1 N HCl/EtOAc (15 mL/15 mL). After partition, the organic layer was washed with water, filtered, and concentrated. The residue was purified by preparative TLC using a mixture of hexane/EtOAc (3:1) as a developing solvent to afford the title compound as a colorless oil (34 mg). ¹H NMR (CDCl₃): δ (ppm) 7.67 (d, 1H, J=7.8 Hz), 7.61 (m, 2H), 7.56 (d, 1H, J=7.9 Hz), 7.37-7.24 (m, 3H), 7.05 (d, 1H, J=7.8 Hz), 7.03 (s, 1H), 4.78 (br. s, 1H), 3.26-3.16 (m, 2H), 1.91 (m, 1H), 1.49 (s, 9H), 1.41 (m, 1H), 1.07-0.96 (m, 2H). ¹³C NMR (CDCl₃): δ (ppm) 155.9, 154.8, 143.2, 130.5, 129.2, 128.8, 128.3, 126.0, 125.8, 124.2, 122.9, 122.4, 120.9, 111.1, 101.3, 79.3, 44.7, 28.4 (3), 23.2, 21.9, 14.3.

### ii) trans-(±)-[2-(3-Benzofuran-2-yl-phenyl)-cyclopropyl]-methylamine Hydrochloride

To a solution of trans-(±)-[2-(3-Benzofuran-2-yl-phenyl)-cyclopropylmethyl]-carbamic acid tert-butyl ester (30 mg, 0.083 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.248 mL, 0.248 mmol) was added to the solution. The mixture was stand at 0° C. for 30 min, and concentrated. The resulting white solid was purified by recrystallization from ethanol/Et₂O to afford the title compound as a white solid (21 mg).

### Example 28

### trans-(±)-[2-(4′-Chloro-biphenyl-2-yl)-cyclopropyl]-methylamine Hydrochloride

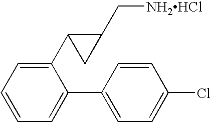

### i) trans-(±)-[2-(2-Bromo-phenyl)-cyclopropylmethyl]-carbamic Acid Tert-Butyl Ester

To a solution of trans-(±)-2-(2-bromo-phenyl)-cyclopropylmethylamine hydrochloride (1.63 g, 6.21 mmol) and Boc₂O (1.35 g, 8.07 mmol) in Et₂O (75 mL) was added 10% aqueous NaOH (14.9 mL, 37.2 mmol) at 0 C. The mixture was stirred at 0° C. for 30 min and then at room temperature for 7 h. To the resulting mixture were added crushed ice and Et₂O. The organic layer was further washed with water (×1), dried over Na₂SO₄ and concentrated under vacuum. The residue was purified by, silica gel chromatography using a mixture of hexane/EtOAc (10:1) as an eluent to afford the title compounds as colorless oils (1.71 g). ¹H NMR (CDCl₃): δ (ppm) 7.44 (d, 2H, J=8.5 Hz), 7.40 (d, 2H, J=8.5 Hz), 7.30-7.19 (m, 3H), 7.00 (d, 1H, J=7.2 Hz), 4.45 (br. s, 1H), 3.05-2.95 (m, 2H), 1.76 (m, 1H), 1.46 (s, 9H), 1.26 (m, 1H), 0.94 (m, 1H), 0.77 (m, 1H). ¹³C NMR (CDCl₃): δ (ppm) 156.2, 141.6, 140.5, 139.6, 133.4, 131.2 (2H), 130.0, 128.8 (2H), 128.2, 126.1, 125.3, 79.6, 44.9, 28.8 (3H), 23.3, 20.6, 14.3.

### ii) trans-(±)-[2-(4′-Chloro-biphenyl-2-yl)-cyclopropylmethyl]-carbamic Acid Tert-Butyl Ester

trans-(±)-[2-(2-bromo-phenyl)-cyclopropylmethyl]-carbamic acid tert-butyl ester (30 mg, 0.092 mmol), Pd(PPh₃)₄ (10.4 mg, 0.009 mmol), and 4-chloro-phenylboronic acid (36.0 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (35.8 mg).

### iii) trans-(±)-[2-(4′-Chloro-biphenyl-2-yl)-cyclopropyl]-methylamine Hydrochloride

To a solution of trans-(±)-[2-(4′-chloro-biphenyl-2-yl)-cyclopropylmethyl]-carbamic acid tert-butyl ester (35 mg, 0.098 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.196 mL, 0.196 mmol) was added to the solution. The mixture was stand at 0° C. for 30 min, and concentrated. The resulting white solid was purified by recrystallization from ethanol/Et₂O to afford the title compound as a white solid (23 mg). ¹H NMR (CDCl₃): δ (ppm) 7.48 (d, 2H, J=6.5 Hz), 7.39 (d, 2H, J=6.5 Hz), 7.34-7.26 (m, 2H), 7.22 (m, 1H), 7.15 (d, 1H, J=7.5 Hz), 2.98-2.93 (m, 1H), 2.58-2.52 (m, 1H), 1.96 (m, 1H), 1.31 (m, 1H), 1.03-0.98 (m, 1H), 0.96-0.91 (m, 1H). ¹³C NMR (CDCl₃): δ (ppm) 143.1, 141.9, 139.5, 134.3, 132.3 (2), 130.8, 129.6 (2), 129.2, 127.6, 127.3, 44.8, 22.2, 20.3, 15.2.

### Example 29

### trans-(±)-[2-(4′-Trifluoromethyl-biphenyl-2-yl)-cyclopropyl]-methylamine Hydrochloride

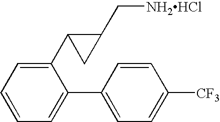

### i) trans-(±)-[2-(4′-Trifluoromethyl-biphenyl-2-yl)-cyclopropylmethyl]-carbamic Acid Tert-Butyl Ester

trans-(±)-[2-(2-bromo-phenyl)-cyclopropylmethyl]-carbamic acid tert-butyl ester (30 mg, 0.123 mmol), Pd(PPh₃)₄ (13.9 mg, 0.012 mmol), and 4-trifluoromethyl-phenylboronic acid (58.2 mg, 0.307 mmol) were dissolved in dimethoxy ethane (DME) (4 mL), and the mixture was degassed for 1 min and stirred for 10 min at room temperature. To the mixture was added 2 M aqueous K₂CO₃ (0.153 mL, 0.307 mmol). The mixture was degassed again for 1 min, and stirred at 85° C. overnight. The resulting mixture was cooled to ambient temperature and poured into a mixture of 5% HCl/EtOAc (30 mL/30 mL). After partition, the organic layer was washed with water, filtered, and concentrated. The residue was purified by preparative TLC using a mixture of hexane/EtOAc (4:1) as a developing solvent to afford the title compound as a colorless oil (48 mg). ¹H NMR (CDCl₃): δ (ppm) 7.73 (d, 2H, J=10.0 Hz), 7.55 (d, 2H, J=7.9 Hz), 7.36-7.21 (m, 3H), 7.01 (d, 1H, J=7.4 Hz), 4.51 (br. s, 1H), 3.03 (m, 2H), 1.74 (m, 1H), 1.45 (s, 9H), 1.28 (m, 1H), 0.94 (m, 1H), 0.79 (m, 1H). ¹³C NMR (CDCl₃): δ (ppm) 156.2, 145.8, 141.4, 130.3 (2), 130.0, 128.6, 126.2, 125.6, 125.5, 125.2, 122.9, 79.7, 44.8, 28.8 (3), 23.4, 20.5, 14.8.

### ii) trans-(±)-[2-(4′-Trifluoromethyl-biphenyl-2-yl)-cyclopropyl]-methylamine Hydrochloride

To a solution of trans-(±)-[2-(4′-trifluoromethyl-biphenyl-2-yl)-cyclopropylmethyl]-carbamic acid tert-butyl ester (45 mg, 0.115 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.230 mL, 0.230 mmol) was added to the solution. The mixture was stand at 0° C. for 30 min, and concentrated. The resulting white solid was purified by recrystallization from ethanol/Et₂O to afford the title compound as a white solid (33 mg). ¹H NMR (CDCl₃): δ (ppm) 7.78 (d, 2H, J=7.9 Hz), 7.61 (d, 2H, J=7.9 Hz), 7.39-7.25 (m, 3H), 7.18 (d, 1H, J=7.6 Hz), 2.92 (dd, 1H, J=5.9, 13.1 Hz), 2.57 (dd, 1H, J=9.0, 13.1 Hz), 1.96 (m, 1H), 1.35 (m, 1H), 1.02-0.98 (m, 1H), 0.96-0.92 (m, 1H). ¹³C NMR (CDCl₃): δ (ppm) 142.8, 139.4, 131.3 (2), 130.7, 129.5, 127.6, 127.2, 126.2, 44.6, 22.1, 20.2, 15.3.

### Example 30

### trans-(±)-[2-(4′-Fluoro-biphenyl-2-yl)-cyclopropyl]-methylamine Hydrochloride

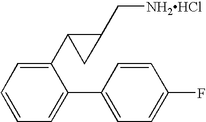

### i) trans-(±)-[2-(4′-Fluoro-biphenyl-2-yl)-cyclopropylmethyl]-carbamic Acid Tert-Butyl Ester

trans-(±)-[2-(2-bromo-phenyl)-cyclopropylmethyl]-carbamic acid tert-butyl ester (30 mg, 0.092 mmol), Pd(PPh₃)₄ (10.4 mg, 0.009 mmol), and 4-fluoro-phenylboronic acid (36.0 mg, 0.23 mmol) were dissolved in dimethoxy ethane (DME) (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 85° C. overnight. The resulting mixture was cooled to ambient temperature and poured into a mixture of 0.1 N HCl/EtOAc (15 mL/15 mL). After partition, the organic layer was washed with water, filtered, and concentrated. The residue was purified by preparative TLC using a mixture of hexane/EtOAc (4:1) as a developing solvent to afford the title compound as a colorless oil (33 mg).

### ii) trans-(±)-[2-(4′-Fluoro-biphenyl-2-yl)-cyclopropyl]-methylamine Hydrochloride

To a solution of trans-(±)-[2-(4′-fluoro-biphenyl-4-yl)-cyclopropylmethyl]-carbamic acid tert-butyl ester (30 mg) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.3 mL) was added to the solution. The mixture was stand at 0° C. for 30 min, and concentrated. The resulting white solid was purified by recrystallization from ethanol/Et₂O to afford the title compound as a white solid (16 mg).

### Example 31

### trans-(±)-N-[2-(2-Benzofuran-2-yl-phenyl)-cyclopropyl]-methylamine Hydrochloride

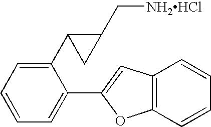

### i) [2-(2-Benzofuran-2-yl-phenyl)-cyclopropylmethyl]-carbamic Acid Tert-Butyl Ester

trans-(±)-[2-(2-bromo-phenyl)-cyclopropylmethyl]-carbamic acid tert-butyl ester (30 mg, 0.092 mmol), Pd(PPh₃)₄ (10.4 mg, 0.009 mmol), and 2-benzofuranboronic acid (37.3 mg, 0.23 mmol) were dissolved in DMF (4 mL), and the mixture was degassed for 1 min and stirred for 10 min at room temperature. To the mixture was added 2 M aqueous K₂CO₃ (0.115 mL, 0.23 mmol). The mixture was degassed again for 1 min, and stirred at 100° C. overnight. The resulting mixture was cooled to ambient temperature and poured into a mixture of 0.1 N HCl/EtOAc (15 mL/15 mL). After partition, the organic layer was washed with water, filtered, and concentrated. The residue was purified by preparative TLC using a mixture of hexane/EtOAc (3:1) as a developing solvent to afford the title compound as a colorless oil (37 mg). ¹H NMR (CDCl₃): δ (ppm) 7.76 (br. d, 1H), 7.67 (d, 1H, J=7.5 Hz), 7.61 (d, 1H, J=8.2 Hz), 7.36-7.27 (m, 4H), 7.11 (d, 1H, J=6.9 Hz), 7.03 (s, 1H), 3.34 (m, 1H), 3.17 (m, 1H), 2.19 (m, 1H), 1.48 (s, 9H), 1.41 (m, 1H), 1.05-1.02 (m, 1H), 1.01-0.93 (m, 1H). ¹³C NMR (CDCl₃): δ (ppm) 155.8, 154.7, 140.0, 130.8, 129.0, 128.9 (3), 126.1, 126.0, 125.8, 124.3, 122.9, 121.0, 120.9, 111.1, 105.5, 79.2, 44.8, 28.4 (3), 22.6, 21.0, 13.5.

### ii) trans-(±)-N-[2-(2-Benzofuran-2-yl-phenyl)-cyclopropyl]-methylamine Hydrochloride

To a solution of trans-(±)-[2-(2-benzofuran-2-yl-phenyl)-cyclopropylmethyl]-carbamic acid tert-butyl ester (35 mg, 0.096 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.289 mL, 0.289 mmol) was added to the solution. The mixture was stand at 0° C. for 30 min, and concentrated. The resulting white solid was purified by recrystallization from ethanol/Et₂O to afford the title compound as a white solid (22 mg).

### Example 32

### trans-(±)-N-[3-(2-Aminomethyl-cyclopropyl)-phenyl]-benzamide Hydrochloride

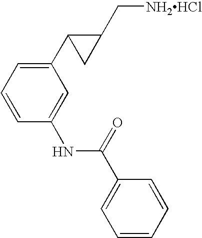

### i) trans-(±)-[2-(3-Amino-phenyl)-cyclopropylmethyl]-carbamic Acid Tert-Butyl Ester

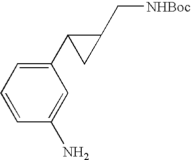

To a mixture of Pd(OAc)₂ (8.3 mg, 0.037 mmol), BINAP (45.8 mg, 0.074 mmol), and trans-(±)-[2-(3-bromo-phenyl)-cyclopropylmethyl]-carbamic acid tert-butyl ester (120 mg, 0.368 mmol) in toluene (4 mL) were added benzophenone imine (0.124 mL, 0.736 mmol) and Cs₂CO₃ (300 mg, 0.92 mmol) under nitrogen. The resulting mixture was stirred at 100° C. overnight, diluted with EtOAc, filtered, and concentrated. The residue was purified with silica gel column chromatography using EtOAc/hexane (1:5) as an eluent to afford the diphenyl ketimine adduct as a yellowish solid. ¹H NMR (CDCl₃): δ (ppm) 7.07 (t, 1H, J=7.8 Hz), 6.56 (dd, 1H, J=1.4 Hz, 7.9 Hz), 6.52 (d, 1H, J=7.6 Hz), 6.46 (s, 1H), 4.70 (br. s, 1H), 3.22 (m, 1H), 3.10 (m, 1H), 1.73 (m, 1H), 1.47 (s, 9H), 1.28 (m, 1H), 0.91 (m, 1H). ¹³C NMR (CDCl₃): δ (ppm) 155.5, 144.9, 143.6, 128.9 (2), 116.4, 112.7, 44.4, 28.0 (3), 22.4, 21.5, 13.8.

To a solution of the ketimine adduct (80 mg, 0.188 mmol) in MeOH (1.9 mL) at room temperature were added NaOAc (36.9 mg, 0.450 mmol) and NH₂OH.HCl (23.5 mg, 0.338 mmol). The mixture was stirred at room temperature for 1 h, diluted with dichloromethane, and purified by preparative TLC using a mixture of hexane/EtOAc (2:1) as a developing solvent to afford the title compound as a colorless oil (33 mg).

### ii) trans-(±)-[2-(3-Benzoylamino-phenyl)-cyclopropylmethyl]-carbamic Acid Tert-Butyl Ester

To a solution of trans-(±)-[2-(3-Amino-phenyl)-cyclopropylmethyl]-carbamic acid tert-butyl ester (7.0 mg, 0.0234 mmol) in dichloromethane (0.3 mL) were added 4-dimethylaminopyridine (DMAP) (3.3 mg, 0.0267 mmol) and benzoyl chloride (0.0047 mL, 0.040 mmol) at 0° C. The mixture was stirred at room temperature overnight and directly purified by preparative TLC using a mixture of hexane/EtOAc (2:1) as a developing solvent to afford the title compound as a colorless oil (8.3 mg, 97%). ¹H NMR (CDCl₃): δ (ppm) 7.89 (m, 3H), 7.57-7.47 (m, 3H), 7.42 (m, 2H), 7.26 (m, 1H), 6.86 (d, 1H, J=7.7 Hz), 4.73 (br. s, 1H), 3.19-3.11 (m, 2H), 1.82 (m, 1H), 1.47 (s, 9H), 1.33 (m, 1H), 1.00-0.89 (m, 2H). ¹³C NMR (CDCl₃): δ (ppm) 166.1, 156.3, 144.2, 138.5, 135.4, 132.2, 129.4, 129.2 (2), 127.4 (2) 122.5, 118.0, 117.9, 79.7, 45.1, 28.8 (3), 23.5, 22.3, 14.9.

### iii) trans-(±)-N-[3-(2-Aminomethyl-cyclopropyl)-phenyl]-benzamide Hydrochloride

To a solution of trans-(±)-[2-(3-Benzoylamino-phenyl)-cyclopropylmethyl]-carbamic acid tert-butyl ester (7 mg, 0.019 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.057 mL, 0.057 mmol) was added to the solution. The mixture was stand at 0° C. for 30 min, and concentrated. The resulting white solid was purified by recrystallization from ethanol/Et₂O to afford the title compound as a white solid (4.3 mg). ¹H NMR (CDCl₃): [symbol] (ppm) 7.94 (d, 2H, J=7.2 Hz), 7.62-7.51 (m, 4H), 7.13-7.09 (m, 2H), 7.41 (d, 1H, J=8.1 Hz), 7.28 (t, 1H, J=7.8 Hz), 6.97 (d, 1H, J=7.6 Hz), 3.02 ((d, 2H, J=7.4 Hz)), 2.06 (m, 1H), 1.46 (m, 1H), 1.17 (m, 1H), 1.10 (m, 1H). ¹³C NMR (CDCl₃): δ (ppm) 169.1, 143.6, 140.1, 136.4, 133.1, 130.0, 129.8 (2), 128.7 (2), 123.6, 120.2, 120.0, 45.0, 23.4, 21.1, 15.2.

### Example 33

### trans-(±)-N-[3-(2-Aminomethyl-cyclopropyl)-phenyl]-acetamide Hydrochloride

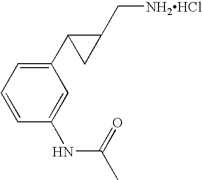

### i) trans-(±)-[2-(3-Acetylamino-phenyl)-cyclopropylmethyl]-carbamic Acid Tert-Butyl Ester

To a solution of trans-(±)-[2-(3-Amino-phenyl)-cyclopropylmethyl]-carbamic acid tert-butyl ester (7.4 mg, 0.0282 mmol) and DMAP (3.5 mg, 0.0282 mmol) in dichloromethane (0.3 mL) was added acetic anhydride (0.004 mL, 0.0423 mmol) at 0° C. The resulting mixture was stirred at 0° C. for 1 h and at room temperature overnight and directly purified by preparative TLC using a mixture of hexane/EtOAc (1:1) as a developing solvent to afford the title compound as a colorless oil (8.1 mg). ¹H NMR (CDCl₃): δ (ppm) 7.28 (br. s, 1H), 7.24-7.18 (m, 3H), 6.80 (d, 1H, J=7.4 Hz), 4.73 (br. s, 1H), 3.18-3.13 (m, 2H), 2.18 (s, 3H), 1.78 (m, 1H), 1.46 (s, 9H), 1.27 (m, 1H), 0.91 (m, 2H). ¹³C NMR (CDCl₃): δ (ppm) 168.3, 155.9, 143.6, 138.0, 128.9, 121.7, 117.3, 117.2, 79.2, 44.6, 28.4 (3), 24.6, 23.0, 21.8, 14.4.

### ii) trans-(±)-N-[3-(2-Aminomethyl-cyclopropyl)-phenyl]-acetamide Hydrochloride

To a solution of trans-(±)-[2-(3-Acetylamino-phenyl)-cyclopropylmethyl]-carbamic acid tert-butyl ester (7 mg, 0.023 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.069 mL, 0.069 mmol) was added to the solution. The mixture was stand at 0° C. for 30 min, and concentrated. The resulting white solid was purified by recrystallization from ethanol/Et₂O to afford the title compound as a white solid (3.3 mg). ¹H NMR (methanol-d₄): [symbol] (ppm) 7.47 (s, 1H), 7.25-7.19 (m, 2H), 6.90 (d, 1H, J=6.4 Hz), 3.01 (d, 2H, J=7.4 Hz), 2.13 (s, 3H), 2.01 (m, 1H), 1.42 (m, 1H), 1.10 (m, 2H). ¹³C NMR (methanol-d₄): δ (ppm) 171.9, 143.6, 140.2, 130.0, 123.0, 119.0, 118.9, 45.0, 24.0, 23.4, 21.1, 15.2.

### Example 34

### trans-(±)-[3-(2-Aminomethyl-cyclopropyl)-phenyl]-benzylamine Hydrochloride

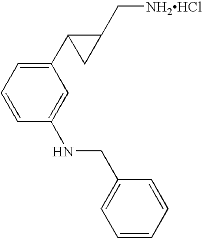

### trans-(±)-[2-(3-Benzylamino-phenyl)-cyclopropylmethyl]-carbamic Acid Tert-Butyl Ester

trans-(±)-[2-(3-Amino-phenyl)-cyclopropylmethyl]-carbamic acid tert-butyl ester (15 mg, 0.0572 mmol) was dissolved in MeOH (0.5 mL) containing benzaldehyde (0.0052 mL, 0.0512 mmol) at 0° C. To the mixture was added NaCNBH₃ (4.3 mg, 0.0686 mmol). The resulting mixture was stirred at 0° C. for 1 h and at room temperature overnight and directly purified by preparative TLC using a mixture of hexane/EtOAc (1:1) as a developing solvent to afford the title compound as a colorless oil (9.9 mg). ¹H NMR (CDCl₃): δ (ppm) 7.38-7.35 (m, 4H), 7.32-7.28 (m, 1H), 7.08 (t, 1H, J=7.8 Hz), 6.47-6.42 (m, 2H), 6.37 (s, 1H), 4.68 (br. s, 1H), 4.33 (s, 2H), 4.07 (br. s, 1H), 3.26-3.23 (m, 1H), 3.12-3.05 (m, 1H), 1.72 (m, 1H), 1.47 (s, 9H), 1.28 (m, 1H), 0.89 (m, 2H). ¹³C NMR (CDCl₃): □ (ppm) 155.8, 148.2, 143.7, 139.4, 129.2, 128.6 (2), 127.5 (2), 127.2, 114.9, 110.4, 110.2, 79.1, 48.3, 44.8, 28.4 (3), 22.7, 22.1, 14.2.

### ii) trans-(±)-[3-(2-Aminomethyl-cyclopropyl)-phenyl]-benzylamine Hydrochloride

To a solution of trans-(±)-[2-(3-Benzylamino-phenyl)-cyclopropylmethyl]-carbamic acid tert-butyl ester (9 mg, 0.026 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.128 mL, 0.128 mmol) was added to the solution. The mixture was stand at 0° C. for 30 min, and concentrated. The resulting white solid was purified by recrystallization from ethanol/Et₂O to afford the title compound as a white solid (5.6 mg). ¹H NMR (CDCl₃): δ (ppm) 7.45-7.41 (m, 6H), 7.28 (d, 1H, J=8.0 Hz), 7.20-7.18 (m, 2H), 4.60 (s, 2H), 3.03 (m, 2H), 2.11 (m, 1H), 1.48 (m, 1H), 1.17-1.12 (m, 2H). ¹³C NMR (methanol-d₄): δ (ppm) 146.0, 132.4, 131.6 (2), 131.4, 130.9, 130.3 (2), 128.3, 121.7, 121.5, 56.8, 44.7, 23.1, 21.6, 15.6.

### Example 35

### trans-(±)-N-[2-(2-Aminomethyl-cyclopropyl)-phenyl]-benzamide Hydrochloride

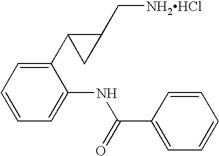

### i) trans-(±)-[2-(2-Amino-phenyl)-cyclopropylmethyl]-carbamic Acid Tert-Butyl Ester

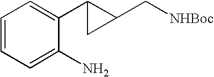

To a mixture of Pd(OAc)₂ (3.4 mg, 0.015 mmol), BINAP (19.1 mg, 0.031 mmol), and trans-(±)-[2-(2-bromo-phenyl)-cyclopropylmethyl]-carbamic acid tert-butyl ester (50 mg, 0.153 mmol) in toluene (3 mL) were added benzophenone imine (0.051 mL, 0.306 mmol) and Cs₂CO₃ (125 mg, 0.383 mmol) under nitrogen. The resulting mixture was stirred at 100° C. overnight, diluted with EtOAc, filtered, and concentrated. The residue was purified by preparative TLC using a mixture of hexane/EtOAc (3:1) as a developing solvent to afford the corresponding diphenyl ketimine compound as a colorless oil (51.9 mg). ¹H NMR (CDCl₃): δ (ppm) 7.50 (d, 2H, J=6.9 Hz), 7.46 (m, 3H), 7.28 (m, 3H), 7.16 (m, 2H), 6.86 (m, 2H), 6.79 (m, 1H), 6.39 (m, 1H), 4.88 (br. s, 1H), 3.34-3.23 (m, 2H), 1.87 (m, 1H), 1.45 (s, 9H), 1.28 (m, 1H), 0.93 (m, 1H), 0.85 (m, 1H). ¹³C NMR (CDCl₃): δ (ppm) 168.3, 156.2, 150.9, 139.9, 136.7, 131.1, 129.8 (2), 129.5 (2), 129.1, 128.7, 128.3, 127.8, 126.1, 123.8, 119.8, 79.4, 45.4, 28.9 (3), 22.9, 18.8, 12.9.

To a solution of the ketimine adduct (35 mg, 0.188 mmol) in MeOH (0.8 mL) at room temperature were added NaOAc (16.2 mg, 0.197 mmol) and NH₂OH.HCl (10.3 mg, 0.148 mmol). The mixture was stirred at room temperature for 2 h, diluted with dichloromethane, and purified by preparative TLC using a mixture of hexane/EtOAc (2:1) as a developing solvent to afford the title compound as a colorless oil (20.5 mg). ¹H NMR (CDCl₃): δ (ppm) 7.05 (m, 1H), 6.98 (d, 1H, J=7.3 Hz), 6.69 (m, 2H), 4.89 (br. s, 1H), 3.42-3.36 (m, 1H), 3.16-3.11 (m, 1H), 1.65 (m, 1H), 1.48 (s, 9H), 1.14 (m, 1H), 0.94 (m, 1H), 0.80 (m, 1H). ¹³C NMR (CDCl₃): δ (ppm) 156.3, 146.3, 127.8, 127.2, 125.5, 117.9, 114.6, 79.4, 44.2, 28.4 (3), 20.2, 14.1, 9.7.

### ii) trans-(±)-[2-(2-Benzoylamino-phenyl)-cyclopropylmethyl]-carbamic Acid Tert-Butyl Ester

To a solution of trans-(±)-[2-(2-Amino-phenyl)-cyclopropylmethyl]-carbamic acid tert-butyl ester (5.8 mg, 0.0221 mmol) in dichloromethane (0.3 mL) were added DMAP (2.7 mg, 0.0221 mmol) and benzoyl chloride (0.0039 mL, 0.0332 mmol) at 0° C. The mixture was stirred at room temperature overnight and directly purified by preparative TLC using a mixture of hexane/EtOAc (1:1) as a developing solvent to afford the title compound as a colorless oil (8.9 mg, 100%). ¹H NMR (CDCl₃): δ (ppm) 8.71 (br. s, 1H), 8.09 (d, 1H, J=7.5 Hz), 8.04 (d, 1H, J=6.6 Hz), 7.60-7.47 (m, 3H), 7.27 (m, 1H), 7.25 (br. s, 1H), 7.12 (t, 1H, J=7.3 Hz), 4.97 (br.s, 1H), 3.33 (m, 1H), 3.10-3.07 (m, 1H), 1.91 (m, 1H), 1.42 (s, 9H), 1.29 (m, 1H), 0.99-0.91 (m, 2H). ¹³C NMR (CDCl₃): δ (ppm) 165.9, 156.3, 136.9, 135.0, 132.4, 131.8, 128.7, 127.4, 126.9, 126.4, 124.8, 122.7, 79.5, 44.3, 28.3 (3), 21.9, 18.1, 11.1.

### iii) trans-(±)-N-[2-(2-Aminomethyl-cyclopropyl)-phenyl]-benzamide Hydrochloride

To a solution of trans-(±)-[2-(2-benzoylamino-phenyl)-cyclopropylmethyl]-carbamic acid tert-butyl ester (8 mg, 0.022 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.065 mL, 0.065 mmol) was added to the solution. The mixture was stand at 0° C. for 30 min, and concentrated. The resulting white solid was purified by recrystallization from ethanol/Et₂O to afford the title compound as a white solid (3.5 mg).

### Example 36

### trans-(±)-1-[2-(2-Aminomethyl-cyclopropyl)-phenyl]-3-(4-chloro-phenyl)-urea Hydrochloride

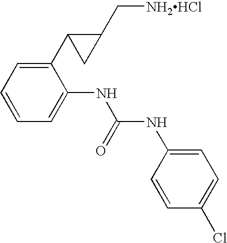

### i) trans-(±)-(2-{2-[3-(4-Chloro-phenyl)-ureido]-phenyl}-cyclopropylmethyl)-carbamic Acid Tert-Butyl Ester

To a solution of trans-(±)-[2-(2-Amino-phenyl)-cyclopropylmethyl]-carbamic acid tert-butyl ester (30.0 mg, 0.114 mmol) in THF (5 mL) were added DMAP (2.8 mg, 0.023 mmol) and 4-chlorophenyl isocyanate (0.022 mL, 0.172 mmol) at room temperature. The mixture was stirred at 50° C. overnight and directly purified by preparative TLC using a mixture of hexane/EtOAc (1:1) as a developing solvent to afford the title compound as a colorless oil (29.1 mg, 61.4%). ¹H NMR (CDCl₃): δ (ppm) 8.42 (br. s, 1H), 8.32 (d, 1H, J=8.2 Hz), 8.08 (br. S, 1H), 7.53 (d, 2H, J=8.8 Hz), 7.29-7.22 (m, 3H), 7.04 (t, 1H, J=6.9 Hz), 6.90 (d, 1H, J=6.9 Hz), 6.95 (t, 1H, J=7.2 Hz), 4.98 (br. t, 1H, J=6.2 Hz), 3.93 (m, 1H), 3.23 (m, 1H), 1.61 (m, 1H), 1.50 (s, 9H), 1.19 (m, 1H), 0.83-0.72 (m, 2H). ¹³C NMR (CDCl₃): δ (ppm) 158.5, 153.3, 139.7, 138.8, 129.2 (2), 128.9, 127.6, 127.4, 127.1, 122.3, 120.2 (2), 119.3, 81.5, 40.6, 28.8 (3), 21.2, 16.2, 6.5.

### ii) trans-(±)-1-[2-(2-Aminomethyl-cyclopropyl)-phenyl]-3-(4-chloro-phenyl)-urea Hydrochloride

To a solution of trans-(±)-(2-{2-[3-(4-Chloro-phenyl)-ureido]-phenyl}-cyclopropylmethyl)-carbamic acid tert-butyl ester (27 mg, 0.058 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.130 mL, 0.130 mmol) was added to the solution. The mixture was stand at 0° C. for 30 min, and concentrated. The resulting white solid was purified by recrystallization from ethanol/Et₂O to afford the title compound as a white solid (18 mg).

### Example 37

### trans-(±)-1-[2-(2-Aminomethyl-cyclopropyl)-phenyl]-3-(4-trifluoromethyl-phenyl)-urea Hydrochloride

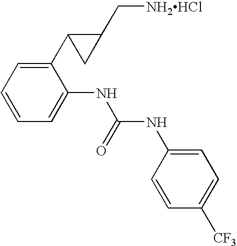

### i) trans-(±)-(2-{2-[3-(4-Trifluoromethyl-phenyl)-ureido]-phenyl}-cyclopropylmethyl)-carbamic Acid Tert-Butyl Ester

To a solution of trans-(±)-[2-(2-Amino-phenyl)-cyclopropylmethyl]-carbamic acid tert-butyl ester (20.0 mg, 0.0762 mmol) in THF (5 mL) were added DMAP (1.9 mg, 0.015 mmol) and 4-trifluoromethylphenyl isocyanate (0.016 mL, 0.114 mmol) at room temperature. The mixture was stirred at 50° C. overnight and directly purified by preparative TLC using a mixture of hexane/EtOAc (1:1) as a developing solvent to afford the title compound as a colorless oil (29.5 mg). ¹H NMR (CDCl₃): δ (ppm) 8.64 (br. s, 1H), 8.34 (d, 1H, J=8.1 Hz), 8.16 (br. S, 1H), 7.69 (d, 2H, J=8.5 Hz), 7.56 (d, 2H, J=8.6 Hz), 7.26 (t, 1H, J=7.1 Hz), 7.05 (d, 1H, J=6.8 Hz), 6.97 (t, 1H, J=7.4 Hz), 5.02 (br. t, 1H, J=6.2 Hz), 3.94 (m, 1H), 3.26 (m, 1H), 1.62 (m, 1H), 1.52 (s, 9H), 1.21 (m, 1H), 0.84-0.76 (m, 2H). ¹³C NMR (CDCl₃): δ (ppm) 158.6, 153.1, 143.4, 139.5, 129.0, 127.6, 127.2, 126.5 (2) (³J(C, F)=3.7 Hz), 124.2 (²J(C, F)=32.5 Hz), 122.5, 119.3, 118.3 (2), 81.6, 40.6, 28.8 (3), 21.2, 16.2, 6.5.

### ii) trans-(±)-1-[2-(2-Aminomethyl-cyclopropyl)-phenyl]-3-(4-trifluoromethyl-phenyl)-urea Hydrochloride

To a solution of trans-(±)-(2-{2-[3-(4-trifluoromethyl-phenyl)-ureido]-phenyl}-cyclopropylmethyl)-carbamic acid tert-butyl ester (27 mg, 0.060 mmol) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.120 mL, 0.120 mmol) was added to the solution. The mixture was stand at 0° C. for 30 min, and concentrated. The resulting white solid was purified by recrystallization from ethanol/Et₂O to afford the title compound as a white solid (17 mg).

### Example 38

### trans-(±)-N-[2-(2-Aminomethyl-cyclopropyl)-phenyl]-acetamide Hydrochloride

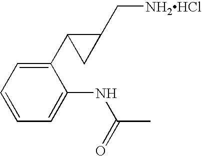

### i) trans-(±)-[2-(3-Acetylamino-phenyl)-cyclopropylmethyl]-carbamic Acid Tert-Butyl Ester

To a solution of trans-(±)-[2-(2-Amino-phenyl)-cyclopropylmethyl]-carbamic acid tert-butyl ester (7.0 mg, 0.0267 mmol) and DMAP (3.3 mg, 0.0267 mmol) in dichloromethane (0.3 mL) was added acetic anhydride (0.004 mL, 0.0423 mmol) at 0° C. The resulting mixture was stirred at 0° C. for 1 h and at room temperature overnight and directly purified by preparative TLC using a mixture of hexane/EtOAc (1:1) as a developing solvent to afford the title compound as a colorless oil (7.2 mg).

### ii) trans-(±)-N-[2-(2-Aminomethyl-cyclopropyl)-phenyl]-acetamide Hydrochloride

To a solution of trans-(±)-[2-(3-acetylamino-phenyl)-cyclopropylmethyl]-carbamic acid tert-butyl ester (6 mg) in CH₂Cl₂ (1 mL) was added TFA (0.1 mL) at 0° C. After standing at 0° C. for 30 min and at room temperature for 1 h, the mixture was concentrated. The residue was dissolved in CH₂Cl₂ (1 mL), and 1 M HCl in Et₂O (0.15 mL) was added to the solution. The mixture was stand at 0° C. for 30 min, and concentrated. The resulting white solid was purified by recrystallization from ethanol/Et₂O to afford the title compound as a white solid (3.6 mg).

### Example 39

### trans-(−)-(2-Phenyl-cyclopropyl)-methylamine Hydrochloride

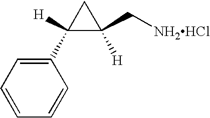

### i) trans-2-Phenyl-cyclopropanecarboxylic acid (2-hydroxy-1-phenyl-ethyl)-amide

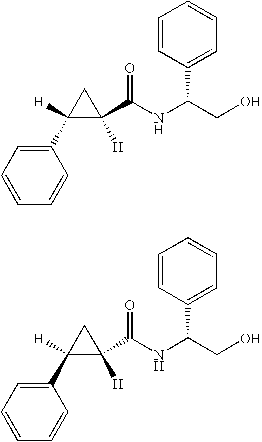

To a stirred solution of trans-(±)-2-Phenyl-cyclopropanecarboxylic acid (1.62 g, 10.0 mmol) in CH₂Cl₂ (20 mL) were added (R)-(−)-2-phenylglycinol (2.06 g, 15.0 mmol), HOBt (1.35 g, 10.0 mmol) and EDC.HCl (2.88 g, 15.0 mmol). The mixture was stirred at 0° C. for 1 h followed at room temperature overnight. The reaction mixture was washed with 5% aqueous citric acid, satd. aqueous NaHCO₃ and satd. NaCl. The organic layer was dried over Na₂SO₄, filtered, and concentrated. The crude residue was purified by silica gel column chromatography using a mixture of EtOAc/Et₂O (1:2) as an eluent to afford the diastereomers as colorless solids. (−)-isomer with higher Rf value (650 mg) and (+)-isomer with lower Rf value (703 mg). higher Rf isomer: ¹H NMR (methanol-d₄): δ (ppm) 7.36-7.13 (m, 9H), 5.03 (t, 1H, J=6.3 Hz), 3.75 (m, 2 H), 2.42 (m, 1H), 2.03 (m, 1H), 1.46 (m, 1H), 1.24 (m, 1H). ¹³C NMR (methanol-d₄): δ (ppm) 173.6, 141.2, 140.4, 128.5 (2), 128.4 (2), 127.4, 127.0 (2), 126.2, 126.0 (2), 65.2, 56.3, 25.7, 24.8, 15.4. lower Rf isomer: ¹H NMR (methanol-d₄): δ (ppm) 7.35-7.10 (m, 9H), 5.02 (t, 1H, J=6.3 Hz), 3.75 (m, 2H), 2.34 (m, 1H), 2.03 (m, 1H), 1.53 (m, 1H), 1.27 (m, 1H). ¹³C NMR (methanol-d₄): δ (ppm) 173.6, 141.2, 140.4, 128.5 (2), 128.4 (2), 127.4, 127.0 (2), 126.3, 126.1 (2H), 65.3, 56.4, 25.8, 24.8, 15.2.

### ii) trans-(−)-2-Phenyl-cyclopropanecarboxylic Acid

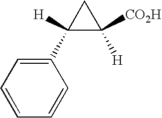

A solution of trans-(−)-2-phenyl-cyclopropanecarboxylic acid (2-hydroxy-1-phenyl-ethyl)-amide (160 mg, 0.569 mmol) in dioxane (5 mL) was added to 3NH₂SO₄ (5 mL). The mixture was stirred at 100° C. for 24 h and then poured into water and extracted with CH₂Cl₂ (×3). The combined organic phases were dried over MgSO₄, filtered and concentrated and all volatiles were removed under vacuum. The crude residue was purified by silica gel column chromatography using a mixture of EtOAc/hexane (1:2) as an eluent to afford the title compound as colorless solid (79 mg). ¹H NMR (CDCl₃): δ (ppm) 9.76 (br. S, 1H), 7.34-7.22 (m, 3H), 7.14 (d, 2H, J=7.0 Hz), 2.64 (m, 1H), 1.93 (m, 1H), 1.70 (m, 1H), 1.44 (m, 1H). ¹³C NMR (CDCl₃): δ (ppm) 180.2, 139.9, 128.9 (2), 127.1, 126.7 (2), 27.5, 24.4, 17.9. (−)-CO₂H, [α]_{D} −387, c 0.61, CHCl₃.

### iii) trans-(−)-(2-Phenyl-cyclopropyl)-methylamine Hydrochloride

To a solution of trans-(−)-2-phenyl-cyclopropanecarboxylic acid (69 mg, 0.425 mmol) in toluene (3 mL) were added dropwise 2 drops of dimethylformamide and thionyl chloride (0.466 mL, 6.38 mmol). After stirring at 80° C. for 2.5 h, the reaction mixture was concentrated under vacuum. The resulting residue was dissolved in toluene (2 mL) again, and the solution was added to liquid ammonia (ca. 10 mL) at −78° C. After stirring at −78° C. for 30 min and then at room temperature for 30 min, CH₂Cl₂ (10 ml) was added to the mixture at −78° C. and the resulting mixture was stirred at room temperature overnight. After addition of EtOAc, the mixture was washed with satd. aqueous NH₄Cl (×2), dried over MgSO₄ and all volatiles were removed under vacuum. The corresponding acid was isolated as pearl yellow powders and further purified by recrystallization from hexane/EtOAc (50 mg).

To a solution of trans-(−)-2-phenyl-cyclopropanecarboxylic acid amide (45 mg, 0.279 mmol) in anhydrous THF (2 mL), was added dropwise 1 M borane/THF solution (1.12 mL) at 0° C. The mixture was heated under reflux at 70° C. overnight, then was quenched by careful addition of 10% aqueous HCl. After stirring at room temperature for 2 h, the THF was removed by evaporation and the residual aqueous solution was washed with Et₂O (×2), neutralized with 10% NaOH, and then extracted with Et₂O (×3). The combined organic layers were dried over MgSO₄ and concentrated until the volume was reduced to about 1 mL. To the solution, was added 1 M HCl in Et₂O (0.56 mL, 0.52 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (38.7 mg). (−)-CH₂NH₂, [α]_{D} −71.3, c 0.45, MeOH

### Example 40

### trans-(±)-(2-Phenyl-cyclopropyl)-methylamine Hydrochloride

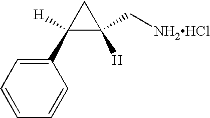

### i) trans-(±)-2-Phenyl-cyclopropanecarboxylic Acid

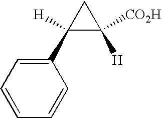

A solution of trans-(±)-2-phenyl-cyclopropanecarboxylic acid (2-hydroxy-1-phenyl-ethyl)-amide (150 mg, 0.534 mmol) in dioxane (5 mL) was added to 3NH₂SO₄ (5 mL). The mixture was stirred at 100° C. for 24 h and then poured into water and extracted with CH₂Cl₂ (×3). The combined organic phases were dried over MgSO₄, filtered and concentrated and all volatiles were removed under vacuum. The crude residue was purified by silica gel column chromatography using a mixture of EtOAc/hexane (1:2) as an eluent to afford the title compound as colorless solid (73 mg, 84%). ¹H NMR (CDCl₃): δ (ppm) 10.3 (br. S, 1H), 7.34-7.22 (m, 3H), 7.14 (d, 2H, J=7.0 Hz), 2.64 (m, 1H), 1.93 (m, 1H), 1.70 (m, 1H), 1.44 (m, 1H). ¹³C NMR (CDCl₃): δ (ppm) 180.2, 139.9, 128.9 (2), 127.1, 126.7 (2), 27.5, 24.4, 17.9. (±)—CO₂H, [α]_{D} +374, c 0.55, CHCl₃.

### ii) trans-(±)-(2-Phenyl-cyclopropyl)-methylamine Hydrochloride

To a solution of trans-(±)-2-phenyl-cyclopropanecarboxylic acid (60 mg, 0.37 mmol) in toluene (3 mL) were added dropwise several drops of dimethylformamide and thionyl chloride (0.403 mL, 5.55 mmol). After stirring at 80° C. for 2.5 h, the reaction mixture was concentrated under vacuum. The resulting residue was dissolved in toluene (2 mL) again, and the solution was added to liquid ammonia (ca. 10 mL) at −78° C. After stirring at −78° C. for 30 min and then at room temperature for 30 min, CH₂Cl₂ (10 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The corresponding amide was isolated as pearl yellow powders and further purified by recrystallization from hexane/EtOAc (45 mg).

To a solution of trans-(±)-2-phenyl-cyclopropanecarboxylic acid amide (42 mg, 0.261 mmol) in anhydrous THF (2 mL), was added dropwise 1 M borane/THF solution (1.04 mL) at 0° C. The mixture was heated under reflux at 70° C. overnight, then was quenched by careful addition of 10% aqueous HCl. After stirring at room temperature for 2 h, the THF was removed by evaporation and the residual aqueous solution was washed with Et₂O (×2), neutralized with 10% NaOH, and then extracted with Et₂O (×3). The combined organic layers were dried over MgSO₄ and concentrated until the volume was reduced to about 1 mL. To the solution, was added 1 M HCl in Et₂O (0.52 mL, 0.52 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (27.2 mg). ¹H NMR (methanol-d₄): δ (ppm) 7.29-7.24 (m, 2H), 7.18-7.13 (m, 3H), 3.01 (d, 2H, J=7.4 Hz), 2.02 (m, 1H), 1.41 (m, 1H), 1.14-1.06 (m, 2H). ¹³C NMR (methanol-d₄): δ (ppm) 141.6, 128.4 (2), 126.0, 125.9 (2), 43.9, 22.2, 19.8, 14.0. (±)—CH₂NH₂, [α]D+72.9, c 0.45, MeOH

### Example 41

### trans-(−)-[2-(3-Bromo-phenyl)-cyclopropyl]-methylamine Hydrochloride

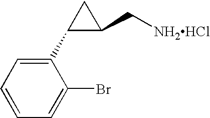

### i) trans-2-(2-Bromo-phenyl)-cyclopropanecarboxylic acid (2-hydroxy-1-phenyl-ethyl)-amide

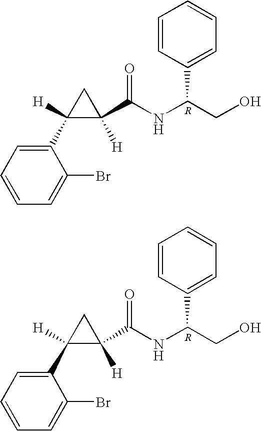

To a stirred solution of trans-(±)-2-(2-Bromo-phenyl)-cyclopropanecarboxylic acid (1.34 g, 5.56 mmol) in CH₂Cl₂ (15 mL) were added (R)-(−)-2-phenylglycinol (1.14 g, 8.34 mmol), HOBt (751 mg, 5.56 mmol) and EDC.HCl (1.60 g, 8.34 mmol). The mixture was stirred at 0° C. for 1 h followed at room temperature overnight. The reaction mixture was washed with 5% aqueous citric acid, satd. aqueous NaHCO₃ and satd. NaCl. The organic layer was dried over MgSO₄, filtered, and concentrated. The crude residue was purified by silica gel column chromatography using a mixture of THF/hexane (1:10) as an eluent to afford the diastereomers as colorless solids. Isomer-1 with higher Rf value (697 mg) and isomer-2 with lower Rf value (711 mg). isomer-2: ¹H NMR (CDCl₃): δ (ppm) 7.55 (d, 1H, J=7.9 Hz), 7.41-7.26 (m, 6H), 7.16-7.10 (m, 1H), 5.06 (m, 1H), 3.82-3.71 (m, 2H), 2.49 (m, 1H), 1.85 (m, 1H), 1.54 (m, 1H), 1.39 (m, 1H).

### ii) trans-(−)-2-(2-Bromo-phenyl)-cyclopropanecarboxylic Acid

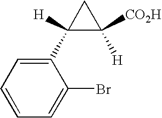

A solution of trans-2-(2-Bromo-phenyl)l-cyclopropanecarboxylic acid (2-hydroxy-1-phenyl-ethyl)-amide (isomer-1 with higher Rf) (450 mg) in dioxane (15 mL) was added to 3N H₂SO₄ (15 mL). The mixture was stirred at 100° C. for 48 h and then poured into water and extracted with CH₂Cl₂ (×3). The combined organic phases were dried over MgSO₄, filtered and concentrated, and all volatiles were removed under vacuum. The crude residue was purified by preparative TLC using a mixture of dichloromethane/MeOH/AcOH (95:5:3) as an eluent to afford the title compound as colorless solid (54 mg). ¹H NMR (CDCl₃): δ (ppm) 11.5 (br. s, 1H), 7.60 (d, 1H, J=7.7 Hz), 7.27 (t, 1H, J=7.4 Hz), 7.13 (t, 1H, J=7.4 Hz), 7.06 (d, 1H, J=7.4 Hz), 2.82 (m, 1H), 1.83 (m, 1H), 1.73 (m, 1H), 1.45 (m, 1H). (−)-CO₂H, [α]_{D} −110.4, c 0.32, CHCl₃.

### iii) trans-(−)-[2-(3-Bromo-phenyl)-cyclopropyl]-methylamine Hydrochloride

To a solution of trans-(+)-2-(2-Bromo-phenyl)-cyclopropanecarboxylic acid (50 mg, 0.208 mmol) in toluene (3 mL) were added dropwise 2 drops of dimethylformamide and thionyl chloride (0.228 mL, 3.12 mmol). After stirring at 80° C. for 2.5 h, the reaction mixture was concentrated under vacuum. The resulting residue was dissolved in toluene (1.5 mL) again, and the solution was added to liquid ammonia (ca. 10 mL) at −78° C. After stirring at −78° C. for 30 min and then at room temperature for 30 min, CH₂Cl₂ (10 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The corresponding amide was isolated as pearl yellow powders and further purified by recrystallization from hexane/EtOAc (35 mg).

To a solution of trans-(+)-2-(2-Bromo-phenyl)-cyclopropanecarboxylic acid amide (28 mg, 0.116 mmol) in anhydrous THF (2.5 mL), was added dropwise 1 M borane/THF solution (0.47 mL) at 0° C. The mixture was heated under reflux at 70° C. overnight, then was quenched by careful addition of 10% aqueous HCl. After stirring at room temperature for 2 h, the THF was removed by evaporation and the residual aqueous solution was washed with Et₂O (×2), neutralized with 10% NaOH, and then extracted with Et₂O (×3). The combined organic layers were dried over MgSO₄ and concentrated until the volume was reduced to about 1 mL. To the solution, was added 1 M HCl in Et₂O (0.23 mL, 0.23 mmol) at 0° C. After stirring at 0° C. for 15 min and at room temperature for 1 h, the mixture was concentrated under vacuum. The resulting residue was purified by HPLC (MeCNHt₂O/TFA) to afford the title compound as a white solid (15 mg). ¹H NMR (methanol-d₄): δ (ppm) 7.57 (d, 1H, J=8.1 Hz), 7.30 (m, 1H), 7.14 (m, 2H), 3.33 (m, 1H), 2.90 (m, 1H), 2.19 (m, 1H), 1.40 (m, 1H), 1.14 (m, 2H). (−)-CH₂NH₂, [α]_{D} −16.2, c 0.21, MeOH.

### Example 42

### trans-(+)-[2-(3-Bromo-phenyl)-cyclopropyl]-methylamine Hydrochloride

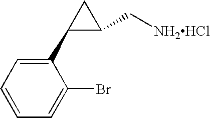

### i) trans-(+)-2-(2-Bromo-phenyl)-cyclopropanecarboxylic Acid

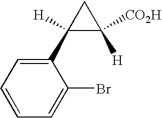

A solution of trans-(+)-2-(2-Bromo-phenyl)l-cyclopropanecarboxylic acid (2-hydroxy-1-phenyl-ethyl)-amide (isomer with lower Rf) (483 mg, 1.34 mmol) in dioxane (15 mL) was added to 3N H₂SO₄ (15 mL). The mixture was stirred at 100° C. for 36 h and then poured into water and extracted with CH₂Cl₂ (×3). The combined organic phases were dried over MgSO₄, filtered and concentrated, and all volatiles were removed under vacuum. The crude residue was purified by preparative TLC using a mixture of dichloromethane/MeOH/AcOH (95:5:3) as an eluent to afford the title compound as colorless solid (87 mg). ¹H NMR (CDCl₃): δ (ppm) 11.5 (br. s, 1H), 7.60 (d, 1H, J=7.7 Hz), 7.27 (t, 1H, J=7.4 Hz), 7.13 (t, 1H, J=7.4 Hz), 7.06 (d, 1H, J=7.4 Hz), 2.82 (m, 1H), 1.83 (m, 1H), 1.73 (m, 1H), 1.45 (m, 1H). (+)-CO₂H, [α]_{D}+109.9, c 0.77, CHCl₃.

### ii) trans-(+)-[2-(3-Bromo-phenyl)-cyclopropyl]-methylamine Hydrochloride

To a solution of trans-(+)-2-(2-Bromo-phenyl)-cyclopropanecarboxylic acid (85 mg, 0.353 mmol) in toluene (3 mL) were added dropwise 2 drops of dimethylformamide and thionyl chloride (0.386 mL, 5.29 mmol). After stirring at 80° C. for 2.5 h, the reaction mixture was concentrated under vacuum. The resulting residue was dissolved in toluene (1.5 mL) again, and the solution was added to liquid ammonia (ca. 10 mL) at −78° C. After stirring at −78° C. for 30 min and then at room temperature for 30 min, CH₂Cl₂ (10 ml) was added to the mixture at −78° C. and the resulting mixture was stirred at room temperature overnight. After addition of EtOAc, the mixture was washed with satd. aqueous NH₄Cl (×2), dried over Na₂SO₄ and all volatiles were removed under vacuum. The corresponding amide was isolated as pearl yellow powders and further purified by recrystallization from hexane/EtOAc (70 mg).

To a solution of trans-(+)-2-(2-Bromo-phenyl)-cyclopropanecarboxylic acid amide (69 mg, 0.287 mmol) in anhydrous THF (2.5 mL), was added dropwise 1 M borane/THF solution (1.15 mL) at 0° C. The mixture was heated under reflux at 70° C. overnight, then was quenched by careful addition of 10% aqueous HCl. After stirring at room temperature for 2 h, the THF was removed by evaporation and the residual aqueous solution was washed with Et₂O (×2), neutralized with 10% NaOH, and then extracted with Et₂O (×3). The combined organic layers were dried over MgSO₄ and concentrated until the volume was reduced to about 1 mL. To the solution, was added 1 M HCl in Et₂O (0.52 mL, 0.52 mmol) at 0° C. After stirring at 0° C. for 15 min and at room temperature for 1 h, the mixture was concentrated under vacuum. The resulting residue was purified by HPLC (MeCNHt₂O/TFA) to afford the title compound as a white solid (46 mg). ¹H NMR (methanol-d₄): δ (ppm) 7.57 (d, 1H, J=8.1 Hz), 7.30 (m, 1H), 7.14 (m, 2H), 3.33 (m, 1H), 2.90 (m, 1H), 2.19 (m, 1H), 1.40 (m, 1H), 1.14 (m, 2H). (+)—CH₂NH₂, [α]_{D} +17.5, c 0.22, MeOH.

### Example 43

### trans-(−)-[2-(4′-Chloro-biphenyl-3-yl)-cyclopropyl]-methylamine Hydrochloride

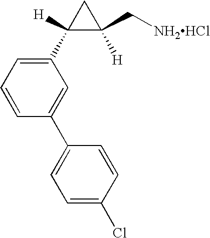

### i) trans-(±)-2-(4′-Chloro-biphenyl-3-yl)-cyclopropanecarboxylic Acid Ethyl Ester

trans-(±)-2-(3-bromo-phenyl)-cyclopropanecarboxylic acid ethyl ester (350 mg, 1.30 mmol), Pd(PPh₃)₄ (150 mg, 0.13 mmol), and 4-chloro-phenylboronic acid (407 mg, 2.60 mmol) were dissolved in dimethoxy ethane (DME) (21 mL), and the mixture was degassed for 1 min and stirred for 10 min at room temperature. To the mixture was added 2 M aqueous K₂CO₃ (1.62 mL, 3.25 mmol). The mixture was degassed again for 1 min, and stirred at 85° C. overnight. The resulting mixture was cooled to ambient temperature and poured into a mixture of 0.1 N HCl/EtOAc (50 mL/50 mL). After partition, the organic layer was washed with water, filtered, and concentrated. the crude residue was purified by silica gel column chromatography using a mixture of EtOAc/hexane (1:3) as an eluent to afford the title compound as colorless solid (352 mg). ¹H NMR (methanol-d₄): δ (ppm) 7.53-7.35 (m, 6H), 7.32 (s, 1H), 7.11 (d, 2H, J=6.8 Hz), 4.21 (q, 2H, J=7.1 Hz), 2.62 (m, 1H), 1.99 (m, 1H), 1.67 (m, 1H), 1.41-1.36 (m, 1H), 1.32 (t, 3H, J=7.1 Hz). ¹³C NMR (methanol-d₄): δ (ppm) 173.7, 141.3, 140.7, 139.9, 133.9, 129.5, 129.3 (2), 128.8 (2), 125.6 (2), 125.5, 61.2, 26.6, 24.7, 17.5, 14.7.

### ii) trans-(±)-2-(4′-Chloro-biphenyl-3-yl)-cyclopropanecarboxylic Acid

A solution of trans-(±)-2-(4′-chloro-biphenyl-3-yl)-cyclopropanecarboxylic acid ethyl ester (400 mg, 1.33 mmol) in MeOH (3 mL) was added to KOH (746 mg, 13.3 mmol) in MeOH (3.5 mL) at 0° C. The mixture was stirred at room temperature overnight and then poured into water and extracted with CH₂Cl₂. The organic layer was discarded and the aqueous phase was acidified with 10% HCl and extracted with CH₂Cl₂ (×2). The combined organic phases were dried over Na₂SO₄ and all volatiles were removed under vacuum. The acid was isolated as white powders and further purified by recrystallization from hexane (355 mg).

### iii) trans-2-(4′-Chloro-biphenyl-3-yl)-cyclopropanecarboxylic Acid (2-hydroxy-1-phenyl-ethyl)-amide

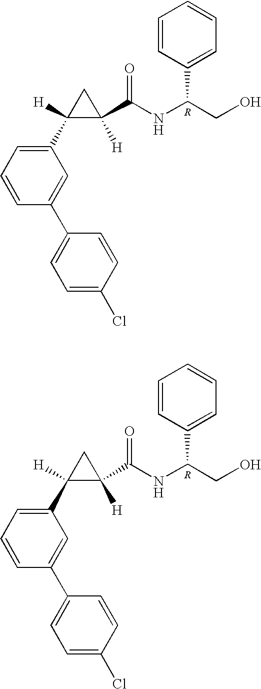

To a stirred solution of trans-(±)-2-(4′-Chloro-biphenyl-3-yl)-cyclopropanecarboxylic acid (350 mg, 1.28 mmol) in CH₂Cl₂ (4 mL) were added (R)-(−)-2-phenylglycinol (264 mg, 1.93 mmol), HOBT (173 mg, 1.28 mmol) and EDC.HCl (370 mg, 1.93 mmol). The mixture was stirred at 0° C. for 1 h followed at room temperature overnight. The reaction mixture was washed with 5% aqueous citric acid, satd. aqueous NaHCO₃ and satd. NaCl. The organic layer was dried over MgSO₄, filtered, and concentrated. The crude residue was purified by silica gel column chromatography using a mixture of EtOAc/hexane (1:1) as an eluent to afford the diastereomers as colorless solids. Isomer-1 with higher Rf value (230 mg) and isomer-2 with lower Rf value (215 mg). higher Rf isomer: ¹H NMR (methanol-d₄): δ (ppm) 7.61 (d, 2H, J=8.6 Hz), 7.44 (d, 2H, J=8.7 Hz), 7.39-7.35 (m, 7H), 7.30-7.27 (m, 1H), 7.15 (d, 1H, J=7.5 Hz), 5.06 (m, 1H), 3.76-3.73 (m, 2H), 2.50 (m, 1H), 2.10 (m, 1H), 1.50 (m, 1H), 1.34-1.28 (m, 1H). ¹³C NMR (methanol-d₄): δ (ppm) 173.1, 141.6, 140.0, 139.9, 139.6, 133.0, 128.7, 128.5 (2), 128.1 (2), 128.0 (2), 127.0, 126.6 (2), 124.9, 124.6, 124.4, 64.8, 55.9, 25.3, 24.4, 15.0. lower Rf isomer: ¹H NMR (DMSO-d₆): δ (ppm) 7.68 (d, 2H, J=8.5 Hz), 7.50 (d, 2H, J=8.5 Hz), 7.47-7.29 (m, 7H), 7.24-7.21 (m, 1H), 7.12 (d, 1H, J=7.6 Hz), 4.90 (m, 2H), 3.56 (m, 2H), 2.27 (m, 1H), 2.17 (m, 1H), 1.26 (m, 1H), 1.03 (m, 1H).

### iv) trans-(−)-2-(4′-Chloro-biphenyl-3-yl)-cyclopropanecarboxylic Acid

A solution of trans-(−)-2-(4′-Chloro-biphenyl-3-yl)-cyclopropanecarboxylic acid (2-hydroxy-1-phenyl-ethyl)-amide (225 mg, 0.574 mmol) in dioxane (6 mL) was added to 3N H₂SO₄ (6 mL). The mixture was stirred at 100° C. for 24 h and then poured into water and extracted with CH₂Cl₂ (×3). The combined organic phases were dried over MgSO₄, filtered and concentrated. All volatiles were removed under vacuum. The crude residue was purified by silica gel column chromatography using a mixture of EtOAc/hexane (1:2) as an eluent to afford the title compound as colorless solid (43 mg).

### v) trans-(−)-[2-(4′-Chloro-biphenyl-3-yl)-cyclopropyl]-methylamine Hydrochloride

To a solution of trans-(−)-2-(4′-Chloro-biphenyl-3-yl)-cyclopropanecarboxylic acid (106 mg, 0.389 mmol) in toluene (3 mL) were added dropwise 2 drops of dimethylformamide and thionyl chloride (0.425 mL, 5.83 mmol). After stirring at 80° C. for 3 h, the reaction mixture was concentrated under vacuum. The resulting residue was dissolved in toluene (1.5 mL) again, and the solution was added to liquid ammonia (ca. 10 mL) at −78° C. After stirring at −78° C. for 30 min and then at room temperature for 30 min, CH₂Cl₂ (10 ml) was added to the mixture at −78° C. and the resulting mixture was stirred at room temperature overnight. After addition of EtOAc, the mixture was washed with satd. aqueous NH₄Cl (×2), dried over MgSO₄ and all volatiles were removed under vacuum. The corresponding carboxylic acid amide was isolated as pearl yellow powders and further purified by recrystallization from hexane/EtOAc (87 mg). (−)-CH₂NH₂, [α]_{D} −50.0, c 0.21, MeOH.

To a solution of trans-(−)-2-(4′-Chloro-biphenyl-3-yl)-cyclopropanecarboxylic acid amide (83 mg, 0.304 mmol) in anhydrous THF (3 mL), was added dropwise 1 M borane/THF solution (1.22 mL) at 0° C. The mixture was heated under reflux at 70° C. overnight, then was quenched by careful addition of 10% aqueous HCl. After stirring at room temperature for 2 h, the THF was removed by evaporation and the residual aqueous solution was washed with Et₂O (×2), neutralized with 10% NaOH, and then extracted with Et₂O (×3). The combined organic layers were dried over MgSO₄ and concentrated until the volume was reduced to about 1 mL. To the solution, was added 1 M HCl in Et₂O (0.608 mL, 0.608 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (63 mg). ¹H NMR (methanol-d₄): [symbol] (ppm) 7.58 (d, 2H, J=8.4 Hz), 7.43 (d, 2H, J=8.1 Hz), 7.40-7.33 (m, 3H), 7.12 (d, 2H, J=7.5 Hz), 3.02 (m, 2H), 2.09 (m, 2H), 1.46 (m, 1H), 1.18 (m, 1H), 1.10 (m, 1H). ¹³C NMR (methanol-d₄): δ (ppm) 143.6, 141.5, 141.2, 134.6, 130.3, 130.1 (2), 129.7 (2), 126.3, 126.0, 125.9, 45.0, 23.4, 21.1, 15.2.

### Example 44

### trans-(+)-[2-(4′-Chloro-biphenyl-3-yl)-cyclopropyl]-methylamine Hydrochloride

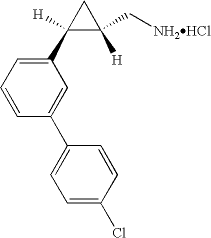

### i) trans-(+)-2-(4′-Chloro-biphenyl-3-yl)-cyclopropanecarboxylic Acid

A solution of trans-2-(4′-Chloro-biphenyl-3-yl)-cyclopropanecarboxylic acid (2-hydroxy-1-phenyl-ethyl)-amide (Low Rf) (210 mg, 0.536 mmol) in dioxane (6 mL) was added to 3N H₂SO₄ (6 mL). The mixture was stirred at 100° C. for 24 h and then poured into water and extracted with CH₂Cl₂ (×3). The combined organic phases were dried over MgSO₄, filtered and concentrated and all volatiles were removed under vacuum. The crude residue was purified by silica gel column chromatography using a mixture of EtOAc/hexane (1:2) as an eluent to afford the title compound as colorless solid (60 mg).

### ii) trans-(+)-[2-(4′-Chloro-biphenyl-3-yl)-cyclopropyl]-methylamine Hydrochloride

To a solution of trans-(+)-2-(4′-Chloro-biphenyl-3-yl)-cyclopropanecarboxylic acid (120 mg, 0.440 mmol) in toluene (3 mL) were added dropwise 2 drops of dimethylformamide and thionyl chloride (0.481 mL, 6.60 mmol). After stirring at 80° C. for 2.5 h, the reaction mixture was concentrated under vacuum. The resulting residue was dissolved in toluene (1.5 mL) again, and the solution was added to liquid ammonia (ca. 10 mL) at −78° C. After stirring at −78° C. for 30 min and then at room temperature for 30 min, CH₂Cl₂ (10 ml) was added to the mixture at −78° C. and the resulting mixture was stirred at room temperature overnight. After addition of EtOAc, the mixture was washed with satd. aqueous NH₄Cl (×2), dried over MgSO₄ and all volatiles were removed under vacuum. The corresponding carboxylic acid amide was isolated as pearl yellow powders and further purified by recrystallization from hexane/EtOAc (118 mg).

To a solution of trans-(+)-2-(4′-Chloro-biphenyl-3-yl)-cyclopropanecarboxylic acid amide (95 mg, 0.350 mmol) in anhydrous THF (3 mL), was added dropwise 1 M borane/THF solution (1.4 mL) at 0° C. The mixture was heated under reflux at 70° C. overnight, then was quenched by careful addition of 10% aqueous HCl. After stirring at room temperature for 2 h, the THF was removed by evaporation and the residual aqueous solution was washed with Et₂O (×2), neutralized with 10% NaOH, and then extracted with Et₂O (×3). The combined organic layers were dried over MgSO₄ and concentrated until the volume was reduced to about 1 mL. To the solution, was added 1 M HCl in Et₂O (0.70 mL, 0.70 mmol) at 0° C. After stirring at 0° C. for 15 min and at room temperature for 1 h, the mixture was concentrated under vacuum. The resulting residue was purified by recrystallization from ethanol/Et₂O to afford the title compound as a white solid (49 mg). ¹H NMR (methanol-d₄): [symbol] (ppm) 7.58 (d, 2H, J=8.4 Hz), 7.43 (d, 2H, J=8.1 Hz), 7.40-7.33 (m, 3H), 7.12 (d, 2H, J=7.5 Hz), 3.02 (m, 2H), 2.09 (m, 2H), 1.46 (m, 1H), 1.18 (m, 1H), 1.10 (m, 1H). ¹³C NMR (methanol-d₄): [symbol] (ppm) 143.5, 141.5, 141.2, 134.6, 130.3, 130.1, 129.7, 126.3, 126.0, 125.9, 45.0, 23.4, 21.1, 15.2. (+)-CH₂NH₂, [α]_{D} +51.1, c 0.20, MeOH.

### Example 45

### trans-(±)-[2-(2,6-Difluoro-phenyl)-cyclopropyl]-methylamine Hydrochloride

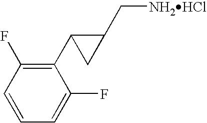

### i) Prop-2-ynyl-carbamic Acid Benzyl Ester

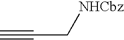

To a solution of propargylamine (5.0 g, 0.106 mol) and triethylamine (19.2 mL, 0.138 mol) in ethyl acetate (125 mL) was added benzyl chloroformate (18.1 mL, 0.127 mol) slowly. The mixture was stirred at room temperature overnight and washed sequentially with 1 N HCl, 5% NaHCO₃, and satd. aqueous NaCl. The solvent was switched to hexane while the product separated as oil (7.2 g). ¹H NMR (CDCl₃): δ (ppm) 7.37 (s, 5H), 5.14 (s, 2H), 74.00 (br. s, 2H), 2.27 (m, 1H). ¹³C NMR (CDCl₃): [symbol] (ppm) 156.3, 136.6, 129.0 (3), 128.6 (2), 80.1, 72.1, 67.5, 31.3.

### ii) 4-Benzyloxycarbonylamino-but-2-en boronic Acid

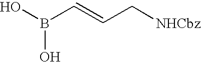

Cyclohexene (3.04 mL, 30.0 mmol) and borane dimethylsulfide complex (94%) (1.42 mL, 15.0 mmol) were dissolved in dry ethyl ether (8 mL). The mixture was stirred at 0° C. for 3 h. Then, the solvent was removed by syringe, and the precipitate was washed with ether. The precipitate was suspended in THF (5 mL). To the mixture was added a solution of prop-2-ynyl-carbamic acid benzyl ester (1.89 g, 10.0 mmol) in THF (5 mL) at 0° C. and the resulting mixture was stirred at room temperature overnight. To the mixture was added trimethylamine N-oxide dihydrate (3.42 g, 30.0 mmol) and the mixture was stirred at room temperature overnight. The mixture was quenched with 2N HCl. The organic layer was separated and the aqueous layer was extracted with ethyl ether. The combined organic layer was extracted with 10% aqueous NaOH (×3). The combined aqueous layer was washed with ether and acidified with 2N HCl and the title compound was obtained as white solid.

### iii) trans-[3-(4,4,5,5-Tetramethyl-[1,3,2]dioxaborolan-2-yl)-allyl]-carbamic Acid Benzyl Ester

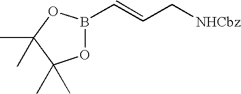

To a solution of trans-4-Benzyloxycarbonylamino-but-2-en boronic acid (1.6 g, 6.81 mmol) in ethyl ether (15 mL) were added pinacol (966 mg, 8.17 mmol) and MgSO₄ (410 mg, 3.40 mmol) at room temperature. The mixture was stirred for 2 h, filtered and concentrated. The residue was purified by silica gel column chromatography using EtOAc/hexane (1:3) as an eluent to afford the title compound as an colorless oil (1.5 g).

### iv) trans-[2-(4,4,5,5-Tetramethyl-[1,3,2]dioxaborolan-2-yl)-cyclopropylmethyl]-carbamic Acid Benzyl Ester

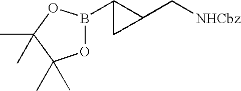

To a solution of Et₂Zn (1.0 M in hexane) (1.0 mL, 1.0 mmol) in dichloromethane (1.0 mL) at −78° C. under nitrogen was added a solution of CH₂I₂ (0.162 mL, 2.0 mmol) in dichloromethane (1.0 mL). After reaction mixture was stirred at −15° C. for 1 h, a solution of [3-(4,4,5,5-Tetramethyl-[1,3,2]dioxaborolan-2-yl)-allyl]-carbamic acid benzyl ester (159 mg, 0.5 mmol) in dichloromethane (1.0 mL) was added. The reaction mixture was stirred at room temperature for 1 h and then quenched with 1 N HCl and ethyl acetate, and the layers were separated. The aqueous layer was extracted with ethyl acetate. The combined organic layer was washed with NaHCO₃ and satd. aqueous NaCl, dried over MgSO₄, filtered and concentrated. The crude residue was purified by silica gel column chromatography using EtOAc/hexane (1:3) as an eluent to afford the title compound as colorless oil (90 mg, 54.3%).

### v) Potassium 2-N-benzyloxycarbonylaminomethyl-cyclopropyltrifluoroborate

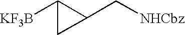

To a solution of trans-[2-(4,4,5,5-Tetramethyl-[1,3,2]dioxaborolan-2-yl)-cyclopropylmethyl]-carbamic acid benzyl ester (75 mg, 0.226 mmol) in MeOH/H₂O (5:1) (1 mL) was added KHF2 (124 mg, 1.59 mmol) at room temperature. After stirring for 5 h, the mixture was concentrated and the residue was extracted with MeCN (×3). The MeCN layer was concentrated and the residue was washed with ethyl ether (×2) and dried to afford the title compound as white solid (54 mg, 77%). ¹H NMR (methanol-d₄): δ (ppm) 7.37-7.32 (m, 5H), 5.07 (s, 2H), 2.97 (m, 2H), 0.76 (m, 1H), 0.29 (m, 1H), 0.02 (m, 1H), −0.63 (m, 1H).

### vi) [2-(2,6-Difluoro-phenyl)-cyclopropylmethyl]-carbamic Acid Benzyl Ester

trans-(±)-[Potassium 2-N-benzyloxycarbonylaminomethyl-cyclopropyltrifluoroborate (60 mg, 0.193 mmol), Pd(PPh₃)₄ (20.2 mg, 0.018 mmol), and 1-bromo-2,6-difluorobenzene (0.022 mL, 0.175 mmol) were dissolved in toluene-H₂O (3:1) (3 mL), and the mixture was degassed for 1 min and stirred for 10 min at room temperature. To the mixture was added K₃PO₄ (149 mg, 0.701 mmol). The mixture was degassed again for 1 min, and stirred at 100° C. overnight. The resulting mixture was cooled to ambient temperature and poured into a mixture of 0.1 N HCl/EtOAc (15 mL/15 mL). After partition, the organic layer was washed with water, dried over MgSO₄, filtered, and concentrated. The residue was purified by preparative TLC using a mixture of hexane/EtOAc (1:1) as a developing solvent to afford the title compound as a colorless oil (15 mg, 27%). ¹H NMR (methanol-d₄): δ (ppm) 7.41-7.32 (m, 5H), 7.14-7.10 (m, 1H), 6.82 (m, 2H), 5.14 (s, 2H), 5.06 (br. S, 1H), 3.46 (m, 1H), 3.18 (m, 1H), 1.71 (m, 1H), 1.53 (m, 1H), 1.21 (m, 1H), 0.94 (m, 1H).

### vii) trans-(±)-[2-(2,6-Difluoro-phenyl)-cyclopropyl]-methylamine Hydrochloride

To a solution of [2-(2,6-Difluoro-phenyl)-cyclopropylmethyl]-carbamic acid benzyl ester (15 mg, mmol) in MeOH (3 mL) were added Pd—C (10%) (15 mg) and 4N HCl in dioxane (2.5 eq.). The mixture was stirred under H₂ (1 atom) at room temperature overnight. The reaction mixture was filtered and the filtrate was concentrated. The residue was purified by recrystallization from MeOH/hexane to afford the title compound as white solid (5.5 mg). ¹H NMR (methanol-d₄): δ 7.31-7.21 (m, 1H), 6.98-6.89 (m, 2H), 3.06-3.03 (m, 2H), 1.93 (m, 1H), 1.64 (m, 1H), 1.26 (m, 1H), 1.13 (m, 1H). ¹³C NMR (methanol-d₄): δ (ppm) 162.5 (2) (¹J(C, F)=246.5 Hz), 128.5 (³J(C, F)=10.7 Hz), 116.5, 111.4 (2) (²J(C, F)=26.1 Hz), 43.9, 17.0, 11.7, 11.6.

### REFERENCES

Setola, V., Dukat, M., Glennon, R. A., Roth, B. L. Molecular determinants for the interaction of the valvulopathic anorexigen norfenfluramine with the 5-HT2B receptor. Mol Pharmacol, 68, 20-33, (2005).
Roth, B. L., Shapiro, D. A. Insights into the structure and function of 5-HT(2) family serotonin receptors reveal novel strategies for therapeutic target development. Expert Opin Ther Targets, 5, 685-695, (2001).
Roth, B. L., Nakaki, T., Chuang, D. M., Costa, E. Aortic recognition sites for serotonin (5HT) are coupled to phospholipase C and modulate phosphatidylinositol turnover. Neuropharmacology, 23, 1223-1225, (1984).
Roth, B. L., McLean, S., Zhu, X. Z., Chuang, D. M. Characterization of two [3H]ketanserin recognition sites in rat striatum. J Neurochem, 49, 1833-1838, (1987).
Roth, B. L., Craigo, S. C., Choudhary, M. S., Uluer, A., Monsma, F. J., Jr., Shen, Y., Meltzer, H. Y., Sibley, D'; R. Binding of typical and atypical antipsychotic agents to 5-hydroxytryptamine-6 and 5-hydroxytryptamine-7 receptors. J Pharmacol Exp Ther, 268, 1403-1410, (1994).
Gray, J. A., Compton-Toth, B. A., Roth, B. L. Identification of two serine residues essential for agonist-induced 5-HT2A receptor desensitization. Biochemistry, 42, 10853-10862, (2003).
Roth, B. L., Sheffler, D. J., Kroeze, W. K. Magic shotguns versus magic bullets: selectively non-selective drugs for mood disorders and schizophrenia. Nat Rev Drug Discov, 3, 353-359, (2004).
Elphick, G. F., Querbes, W., Jordan, J. A., Gee, G. V., Eash, S., Manley, K., Dugan, A., Stanifer, M., Bhatnagar, A., Kroeze, W. K., Roth, B. L., Atwood, W. J. The human polyomavirus, JCV, uses serotonin receptors to infect cells. Science, 306, 1380-1383, (2004).
Choudhary, M. S., Sachs, N., Uluer, A., Glennon, R. A., Westkaemper, R. B., Roth, B. L. Differential ergoline and ergopeptine binding to 5-hydroxytryptamine-2A receptors: ergolines require an aromatic residue at position 340 for high affinity binding [published erratum appears in Mol Pharmacol 1995 September; 48(3):568]. Mol Pharmacol, 47, 450-457, (1995).
Setola, V., Hufeisen, S. J., Grande-Allen, K. J., Vesely, I., Glennon, R. A., Blough, B., Rothman, R. B., Roth, B. L. 3,4-Methylenedioxymethamphetamine (MDMA, “Ecstasy”) Induces Fenfluramine-Like Proliferative Actions on Human Cardiac Valvular Interstitial Cells in Vitro. Mol Pharmacol, 63, 1223-1229, (2003).
Launay J M, Herve P, Peoc'h K, Tournois C, Callebert J, Nebigil C G, Etienne N, Drouet L, Humbert, Simonneau G, Maroteaux L. (2002) Nat. Med. “Function of the serotonin 5-hydroxytryptamine 2B receptor in pulmonary hypertension,” October: 8(10):1129-1135.
Rauser, L., Savage, J. E., Meltzer, H. Y., Roth, B. L. Inverse agonist actions of typical and atypical antipsychotic drugs at the human 5-hydroxytryptamine(2C) receptor. J Pharmacol Exp Ther, 299, 83-89., (2001).
Bhatnagar, A., Sheftler, D. J., Kroeze, W. K., Compton-Toth, B., Roth, B. L. Caveolin-1 interacts with 5-HT2A serotonin receptors and profoundly modulates the signaling of selected Galphaq-coupled protein receptors. J Biol Chem, 279, 34614-34623, (2004).
Choudhary, M. S., Craigo, S., Roth, B. L. Identification of receptor domains that modify ligand binding to 5-hydroxytryptamine-2 and 5-hydroxytryptaminelc serotonin receptors. Mol Pharmacol, 42, 627-633, (1992).
Rothman, R. B., Baumann, M. H., Savage, J. E., Rauser, L., McBride, A., Hufeisen, S. J., Roth, B. L. Evidence for possible involvement of 5-HT(2B) receptors in the cardiac valvulopafhy associated with fenfluramine and other serotonergic medications. Circulation, 102, 2836-2841., (2000).
Glennon, R. A., Lee, M., Rangisetty, J. B., Dukat, M., Roth, B. L., Savage, J. E., McBride, A., Rauser, L., Hufeisen, S., Lee, D. K. 2-Substituted tryptamines: agents with selectivity for 5-HT(6) serotonin receptors. J Med Chem, 43, 1011-1018, (2000).
Kohen, R., Metcalf, M. A., Khan, N., Druck, T., Huebner, K., Lachowicz, J. E., Meltzer, H. Y., Sibley, D. R., Roth, B. L., Hamblin, M. W. Cloning, characterization, and chromosomal localization of a human 5-HT6 serotonin receptor. J Neurochem, 66, 47-56, (1996).
Roth, B. L., Nakaki, T., Chuang, D. M., Costa, E. 5-Hydroxytryptamine-2 receptors coupled to phospholipase C in rat aorta: modulation of phosphoinositide turnover by phorbol ester. J Pharmacol Exp Ther, 238, 480-485, (1986).
Gray, J. A., Sheffler, D. J., Bhatnagar, A., Woods, J. A., Hufeisen, S. J., Benovic, J. L., Roth, B. L. Cell-type specific effects of endocytosis inhibitors on 5-hydroxytryptamine(2A) receptor desensitization and resensitization reveal an arrestin-, GRK2-, and GRK5-independent mode of regulation in human embryonic kidney 293 cells. Mol Pharmacol, 60, i 020-1030., (2001).
Gray, J. A., Bhatnagar, A., Gurevich, V. V., Roth, B. L. The interaction of a constitutively active arrestin with the arrestin-insensitive 5-HT(2A) receptor induces agonist-independent internalization. Mol Pharmacol, 63, 961-972, (2003).
Xia, Z., Gray, J. A., Compton-Toth, B. A., Roth, B. L. A Direct Interaction of PSD-95 with 5-HT2A Serotonin Receptors Regulates Receptor Trafficking and Signal Transduction. J Biol Chem, 278, 21901-21908, (2003).
Chavkin, C., Sud, S., Jin, W., Stewart, J., Zjawiony, J. K., Siebert, D. J., Toth, B. A., Hufeisen, S. J., Roth, B. L. Salvinorin A, an active component of the hallucinogenic sage salvia divinorum is a highly efficacious kappa-opioid receptor agonist: structural and functional considerations. J Pharmacol Exp Ther, 308, 1197-1203, (2004). Davies, M. A., Sheftler, D. J., Roth, B. L. Aripiprazole: a novel atypical antipsychotic drug with a uniquely robust pharmacology. CNS Drug Rev, 10, 317-336, (2004).
Kaiser, C. et al. J. Med. Chem. (1962) “,” 5:1243-1264 and Zirkel et al. J. Med. Chem. (1962) “2-Substituted Cyclopropylamines. Effect Structure upon Monoamine Oxidase-Inhibitory Activity as Measured in Vivo by Potentiation of Tryptamine Convulsions,” 5:1265-1284
Isaac M, Slassi A, O'Brien A, Edwards L, MacLean N, Bueschkens D, Lee D K, McCallum K, De Lannoy I, Demchyshyn L, Kamboj R. Bioorg Med Chem. Lett. Pyrrolo[3,2,1-ij]quinoline derivatives, a 5-HT2c receptor agonist with selectivity over the 5-HT2a receptor: potential therapeutic applications for epilepsy and obesity, 2000 May 1; 10(9):919-21
Teotino, U. et al. J. Med. Chem. (1967) “Chemical and Biological Properties of Some Aminomethyl-2-phenylcyclopropane Derivatives. Pharmacological Comparison with Tranylcypromine,”
Smith B M, Smith J M, Tsai J H, Schultz J A, Gilson C A, Estrada S A, Chen R R, Park D M, Prieto E B, Gallardo C S, Sengupta D, Thomsen W J, Saldana H R, Whelan K T, Menzaghi F, Webb R R, Beeley N R Bioorg Med Chem. Lett. 2005 Discovery and SAR of new benzazepines as potent and selective 5-HT(2C) receptor agonists for the treatment of obesity. March 1; 15(5):1467-70
Kimura Y, Hatanaka K, Naitou Y, Maeno K, Shimada I, Koakutsu A, Wanibuchi F, Yamaguchi T. Pharmacological profile of YM348, a novel, potent and orally active 5-HT2C receptor agonist, Eur J. Pharmacol. 2004 January 1; 483(1):37-43.
Isaac, M. Serotonergic 5-HT2C receptors as a potential therapeutic target for the design antiepileptic drugs. Curr Top Med Chem, 5, 59-67, (2005).
Smith, B. M., Smith, J. M., Tsai, J. H., Schultz, J. A., Gilson, C. A., Estrada, S. A., Chen, R. R., Park, D. M., Prieto, E. B., Gallardo, C. S., Sengupta, D., Thomsen, W. J., Saldana, H. R., Whelan, K. T., Menzaghi, F., Webb, R. R., Beeley, N. R. Discovery and SAR of new benzazepines as potent and selective 5-HT(2C) receptor agonists for the treatment of obesity. Bioorg. Med. Chem. Lett., 15, 1467-1470, (2005).
Isaac, M. Serotonergic 5-HT2C receptors as a potential therapeutic target for the design antiepileptic drugs. Curr Top Med Chem, 5, 59-67, (2005).
Smith, B. M., Smith, J. M., Tsai, J. H., Schultz, J. A., Gilson, C. A., Estrada, S. A., Chen, R. R., Park, D. M., Prieto, E. B., Gallardo, C. S., Sengupta, D., Thomsen, W. J., Saldana, H. R., Whelan, K. T., Menzaghi, F., Webb, R. R., Beeley, N. R.
Discovery and SAR of new benzazepines as potent and selective 5-HT(2C) receptor agonists for the treatment of obesity. Bioorg. Med. Chem. Lett., 15, 1467-1470, (2005).
Burger, A., Yost, W. L. Arylcycloalkylamines. I. 2-Phenylcyclopropylamine. J. Am. Chem. Soc., 70, 2198-2201, (1948).
Teotino, U. M., Bella, D. D., Gandini, A., Benelli, G. Chemical and biological properties of some aminomethyl-2-phenylcyclopropane derivatives. Pharmacological comparison with tranylcypromine. J. Med. Chem., 10, 1091-1096, (1967).
Yoshida, S., Meyer, O. G., Rosen, T. C., Haufe, G., Ye, S., Sloan, M. J., Kirk, K. L. Fluorinated phenylcyclopropylamines. 1. Synthesis and effect of fluorine substitution at the cyclopropane ring on inhibition of microbial tyramine oxidase. J. Med. Chem., 47, 1796-1806, (2004).
Krishnamurthy, S. A highly efficient and general-monomethylation of functionalized primary amines via formylation-borane:methyl sulfide reduction. Tetrahedron Lett., 23, 3315-3318, (1982).
Balboni, G., Salvadori, S., Guerrini, R., Negri, L., Giannini, E., Bryant, S. D., Jinsmaa, Y., Lazarus, L. H. Synthesis and opioid activity of N,N-dimethyl-Dmt-Tic-NH—CH(R)—R′ analogues: acquisition of potent delta antagonism. Bioorg Med Chem, 11, 5435-5441, (2003).
Arvidsson, L. E., Johansson, A. M., Hacksell, U., Nilsson, J. L., Svensson, K., Hjorth, S., Magnusson, T., Carlsson, A., Lindberg, P., Andersson, B., et al. N,N-Dialkylated monophenolic trans-2-phenylcyclopropylamines: novel central 5-hydroxytryptamine receptor agonists. J. Med. Chem., 31, 92-99, (1988).
Kawamoto, H., Ozaki, S., Itoh, Y., Miyaji, M., Arai, S., Nakashima, H., Kato, T., Ohta, H., Iwasawa, Y. Discovery of the first potent and selective small molecule opioid receptor-like (ORL1) antagonist: 1-[(3R,4R)-1-cyclooctylmethyl-3-hydroxymethyl-4-piperidyl]-3-ethyl-1,3-dihydro-2H-benzimidazol-2-one (J-113397). J. Med. Chem., 42, 5061-5063, (1999).
Chandrasekhar, S., Reddy, C. R., Ahmed, M. A Single Step Reductive Amination of Carbonyl Compounds with Polymethylhydrosiloxane-Ti(OiPr)4. Synlett, 1655-1657, (2000).
Miyaura, N., Suzuki, A. Palladium-Catalyzed Cross-Coupling Reactions of Organoboron Compounds. Chem. Rev., 95, 2457-2483, (1995).
Zhang, X., Hodgetts, K., Rachwal, S., Zhao, H., Wasley, J. W., Craven, K., Brodbeck, R., Kieltyka, A., Hoffman, D., Bacolod, M. D., Girard, B., Tran, J., Thurkauf, A. trans-1-[(2-Phenylcyclopropyl)methyl]-4-arylpiperazines: mixed dopamine D(2)/D(4) receptor antagonists as potential antipsychotic agents. J. Med. Chem., 43, 3923-3932, (2000).
Overberger, C. G., Shimokawa, Y. Synthesis and properties of asymmetric polymers. XXIII. Synthesis and optical properties of asymmetric polyamides derived from optically active cyclic dicarboxylic acids. Macromolecules, 9, 718-722, (1971).
Setola, V., Hufeisen, S. J., Grande-Allen, K. J., Vesely, I., Glennon, R. A., Blough, B., Rothman, R. B., Roth, B. L. 3,4-methylenedioxymethamphetamine (MDMA, “Ecstasy”) induces fenfluramine-like proliferative actions on human cardiac valvular interstitial cells in vitro. Mol. Pharmacol., 63, 1223-1229, (2003).
Lipinski, C. A., Lombardo, F., Dominy, B. W., Feeney, P. J. Experimental and Computational Approaches to Estimate Solubility and Permeability in Drug Discovery and Development Settings. Adv. Drug Deliv. Rev., 23, 3-25, (1997).
Lipinski, C. A. Drug-like properties and the causes of poor solubility and poor permeability. J Pharmacol Toxicol Methods, 44, 235-249, (2000).
Ajay. Predicting drug-likeness: why and how? Curr Top Med Chem, 2, 1273-1286, (2002).
Bickerdike, M. J. 5-HT2C Receptor Agonists as Potential Drugs for the Treatment of Obesity. Current Topics in Med. Chem., 3, 885-897, (2003).
Annmarie L. Sabb, R. L. V., Gregory S. Welmaker, Joan E. Sabalski, Joseph Coupet, John Dunlop, Sharon Rosenzweig-Lipson, Boyd Harrison. Cycloalkyl[b][1,4]benzodiazepinoindoles are agonists at the human 5-HT2C receptor. Bioorg. Med. Chem. Lett., 14, 2603-2607, (2004).
Brian M. Smith, J. M. S., James H. Tsai, Jeffrey A. Schultz, Charles A. Gilson, Scott A. Estrada, Rita R. Chen, Douglas M. Park, Emily B. Prieto, Charlemagne S. Gallardo, Dipanjan Sengupta, William J. Thomsen, Hazel R. Saldana, Kevin T. Whelan, Frederique Menzaghi, Robert R. Webb, Nigel R. A. Beeley. Discovery and SAR of new benzazepines as potent and selective 5-HT2C receptor agonists for the treatment of obesity. Bioorg. Med. Chem. Lett., 1467-1470, (2005).
Cacchi, S., Fabrizi, G., Goggiamani, A. Palladium-catalyzed hydroxycarbonylation of aryl and vinyl halides or triflates by acetic anhydride and formate anions. Org Lett, 5, 4269-4272, (2003).
Chambers, J. J., Kurrasch-Orbaugh, D. M., Parker, M. A., Nichols, D. E. Enantiospecific synthesis and pharmacological evaluation of a series of super-potent, conformationally restricted 5-HT(2A/2C) receptor agonists. J. Med. Chem., 44, 1003-1010, (2001).
Capdevielle, P., Maumy, M. Esters are effective co-catalysts in copper-catalyzed methanolysis of aryl bromides. Tetrahedron Lett, 34, 1007-1010, (1993).
Maligres, P. E., M Waters, M. S., Fleitz, F., Askin, D. A highly catalytic robust palladium catalyzed cyanation of aryl bromides. Tetrahedron Lett., 40, 8193-8195, (1999).
Littke, A. F., Schwarz, L., Fu, G. C. Pd/P(t-Bu)(3): a mild and general catalyst for Stille reactions of aryl chlorides and aryl bromides. J. Am. Chem. Soc., 124, 6343-6348, (2002).
DeVasher, R. B., Moore, L. R., Shaughnessy, K. H. Aqueous-phase, palladium-catalyzed cross-coupling of aryl bromides under mild conditions, using water-soluble, sterically demanding alkylphosphines. J. Org. Chem., 69, 7919-7927, (2004).
Fang, G. H., Yan, Z. J., Deng, M. Z. Palladium-catalyzed cross-coupling of stereospecific potassium cyclopropyl trifluoroborates with aryl bromides. Org. Lett., 6, 357-360, (2004).
Biship, M. J., Nilsson, B. M. New 5HT2c receptor agonist. Expert Opinion Ther. Patenet, 13, 1691-1705, (2003).
Chen, H., Deng, M. Z. A Novel Stereocontrolled Synthesis of 1,2-trans Cyclopropyl Ketones via Suzuki-Type Coupling of Acid Chlorides with Cyclopropylboronic Acids. Org. Lett., 2, 1649-1651, (2000).
Wang, M. X., Feng, G. Q., Zheng, Q. Y. Synthesis of high enantiomeric purity gem-dihalocyclopropane derivatives from biotransformation of nitrides and amides. Tetrahedron: Asymmetry, 15, 347-354, (2004).
Xian, Z., Bhutan, R. G., Daniels, D. J., Portuguese, P. S. Interaction of bivalent ligand KDN21 with heterodimeric delta-kappa opioid receptors in human embryonic kidney 293 cells. Mol Pharmacol, 68, 1079-1086, (2005).
Watson, J. A., Elliott, A. C., Brown, P. D. Serotonin elevates intracellular Ca²⁺ in rat choroid plexus epithelial cells by acting on 5-HT2C receptors. Cell Calcium, 17, 120-128, (1995).
Raymond, J. R., Mukhin, Y. V., Gelasco, A., Turner, J., Collinsworth, G., Gettys, T. W., Grewal, J. S., Garnovskaya, M. N. Multiplicity of mechanisms of serotonin receptor signal transduction. Pharmacol Ther, 92, 179-212, (2001).
Lund, P. E., Shariatmadari, R., Uustare, A., Detheux, M., Parmentier, M., Kukkonen, J. P., Akerman, K. E. The orexin OX1 receptor activates a novel Ca²⁺ influx pathway necessary for coupling to phospholipase C. J Biol Chem, 275, 30806-30812, (2000).
Lin, K., Sadee, W., Quillan, J. M. Rapid measurements of intracellular calcium using a fluorescence plate reader. Biotechniques, 26, 318-316, (1999).
Lonart, G., Alagarsamy, S., Ravula, R., Wang, J., Johnson, K. Inhibition of the phospholipase C linked metabotropic glutamate receptor by 2 amino 3 phosphonopropionate is dependent on extracellular calcium. J. Neurochem., 59, 772-775, (1992).
Bubar M J, Seitz P K, Thomas M L, Cunningham K A. (2005) Validation of a selective serotonin 5-HT(2C) receptor antibody for utilization in fluorescence immunohistochemistry studies. Brain Res. 1063:105-13.
Manning T J Jr, Sontheimer H. (1999) Recording of intracellular Ca2+, Cl—, pH and membrane potential in cultured astrocytes using a fluorescence plate reader. J Neurosci Methods. 91:73-81.
Guan X M, Kobilka T S and Kobilka B K (1992) Enhancement of membrane insertion and function in a type IIIb membrane protein following introduction of a cleavable signal peptide. J Biol Chem 267:21995-21998.
Morgenstern J P and Land H (1990) A series of mammalian expression vectors and characterisation of their expression of a reporter gene in stably and transiently transfected cells. Nucleic Acids Res 18:1068.
Rothman R B, Baumann M H, Savage J E, Rauser L, McBride A, Hufeisen S J and Roth B L (2000) Evidence for possible involvement of 5-HT(2B) receptors in the cardiac valvulopathy associated with fenfluramine and other serotonergic medications. Circulation 102:2836-2841.
Shapiro D A, Renock S, Arrington E, Chiodo L A, Liu L X, Sibley D R, Roth B L and Mailman R (2003) Aripiprazole, a novel atypical antipsychotic drug with a unique and robust pharmacology. Neuropsychopharmacology 28:1400-1411.
Zhang J H, Chung T D and Oldenburg K R (1999) A Simple Statistical Parameter for Use in Evaluation and Validation of High Throughput Screening Assays. J Biomol Screen 4:67-73.

1. A method for treating a disorder, condition or undesired symptom which is associated with the 5-HT2C receptor which comprises administering to an individual in need of such treatment a therapeutically effective amount or combined amount of one or more compounds of formula: 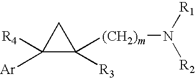
or pharmaceutically acceptable salts, esters, solvates and prodrugs thereof, where:
m is 1 or 2;
R₁ and R₂ are the same or different and are independently selected from H, or C₁-C₆-alkyl groups;
R₃ and R₄ are the same or different and are independently selected from H, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is an aryl group or heteroaryl group which can contain one or more rings at least one of which is aromatic, when the aryl or heteroaryl group contains two or more rings, the rings are either fused or optionally linked by a single bond or a linker group L, wherein L is selected from an alkyl linkage, an olefin linkage, an alkyne linkage, an ether, a thioether linkage, a ketone linkage, an amine linkage, an ester linkage, an amide linkage, a carbamyl linkage or a urea linkage,
wherein at least one of the one or more compounds is a 5-HT2C receptor agonist or selective agonist.

2. (canceled)

3. The method of claim 1 wherein the condition, disease or symptoms is selected from the group consisting of obesity or any complication thereof, gastrointestinal disorders, diabetes, sleep apnea, hypertension, hypertension, hyperlipidemia, an eating disorder, cardiovascular disease, psychiatric disorders, disorders of the central nervous system, damage to the central nervous system, depression, anxiety or panic disorders, obsessive-compulsive disorder, schizophrenia, psychosis, dementia, memory deficit, Parkinson's Disease, Alzheimer's Disease, intellectual deficit associated with Alzheimer's disease, bipolar disorders, adjustment disorders, movement disorders, dystonia, chronic pain, migraine, epilepsy, substance abuse, addiction to alcohol and drugs, and sexual dysfunction in males or females.

4.-17. (canceled)

18. A method for decreasing food intake, inducing satiety, or controlling weight gain of an individual comprising administering to the individual a therapeutically effective amount of a compound or a pharmaceutical composition thereof wherein the compound has the formula: 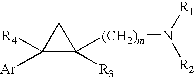
or pharmaceutically acceptable salts, esters, solvates and or prodrugs thereof, where:
m is 1 or 2;
R₁ and R₂ are the same or different and are independently selected from H, or C₁-C₆-alkyl groups;
R₃ and R₄ are the same or different and are independently selected from H, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is an aryl group or heteroaryl group which can contain one or more rings at least one of which is aromatic, when the aryl or heteroaryl group contains two or more rings, the rings are either fused or optionally linked by a single bond or a linker group L, wherein L is selected from an alkanediyl, an olefin linkage, an alkyne linkage, an ether, a thioether linkage, a ketone linkage, an amine linkage, an ester linkage, an amide linkage, a carbamyl linkage or a urea linkage,
wherein the compound is a 5-HT2C receptor agonist or selective agonist.

19.-21. (canceled)

22. A method for or modulating a 5-HT2C receptor in vivo or in vitro which comprises contacting the receptor with one or more compounds having the formula: 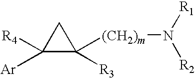
or pharmaceutically acceptable salts, esters, solvates and prodrugs thereof, where:
m is 1 or 2;
R₁ and R₂ are the same or different and are independently selected from H, or C₁-C₆-alkyl groups;
R₃ and R₄ are the same or different and are independently selected from H, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is an aryl group or heteroaryl group which can contain one or more rings at least one of which is aromatic, when the aryl or heteroaryl group contains two or more rings, the rings are either fused or optionally linked by a single bond or a linker group L, wherein L is selected from an alkyl linkage, an olefin linkage, an alkyne linkage, an ether, a thioether linkage, a ketone linkage, an amine linkage, an ester linkage, an amide linkage, a carbamyl linkage or a urea linkage,
wherein the 5-HT2C receptor is activated or stimulated.

23. (canceled)

24. The method of claim 22 wherein the compound is a 5-HT2C receptor agonist or selective agonist.

25.-42. (canceled)

43. The method of claim 22 wherein the compound is selected from the group consisting any one of TKU-II-17 (+/−)trans, TKU-II-113 (+) trans, TKU-II-115 (−)trans; TKU-II-44, TKU-1144, TKU-II-78, TKU-II-90, TKU-II-57, TKU-II-58, TKU-II-81, TKU-II-82, TKU-II-119, TKU-II-120, a salt thereof, an ester thereof, a solvate thereof, a prodrug thereof and a mixture thereof.

44.-45. (canceled)

46. A pharmaceutical composition which comprises a therapeutically effective amount or combined amount of one or more compounds having the formula:
has the formula: 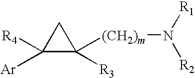
or pharmaceutically acceptable salts, esters, solvates and prodrugs thereof, where:
m is 1 or 2;
R₁ and R₂ are the same or different and are independently selected from H, or C₁-C₆-alkyl groups;
R₃ and R₄ are the same or different and are independently selected from H, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is an aryl group or heteroaryl group which can contain one or more rings at least one of which is aromatic, when the aryl or heteroaryl group contains two or more rings, the rings are either fused or optionally linked by a single bond or a linker group L, wherein L is selected from an alkyl linkage, an olefin linkage, an alkyne linkage, an ether, a thioether linkage, a ketone linkage, an amine linkage, an ester linkage, an amide linkage, a carbamyl linkage or a urea linkage,
wherein the one or more compounds are 5-HT2C receptor agonists or selective agonists.

47. (canceled)

48. The composition of claim 46 wherein the compound is a 5-HT2C receptor selective agonist.

49. The composition of claim 46 wherein in the compound R₁ and R₂ are both hydrogen.

50. (canceled)

51. The composition of claim 46 wherein in the compound R₃ and R₄ are both hydrogens.

52.-55. (canceled)

56. The composition of any one of claims 46-55 wherein Ar is a phenyl substituted with a phenyl, a furanyl or a benzofuranyl group.

57.-62. (canceled)

63. The composition of claim 46 wherein the compound is in the trans configuration with respect to Ar and the —(CH₂)ₘ—NR₁R₂ group.

64. The composition of claim 46 wherein m is 1.

65. The composition of claim 46 wherein the compound is a (+) enantiomer substantially free of the corresponding (−) enantiomer.

66. The composition of claim 46 wherein the compound is selected from the group consisting of TKU-II-17 (+/−)trans, TKU-II-113 (+) trans, TKU-II-115 (−)trans; TKU-1144, TKU-1144, TKU-II-78, TKU-1′-90, TKU-II-57, TKU-II-58, TKU-II-81, TKU-II-82, TKU-II-119, TKU-II-120, salts thereof, esters thereof, solvates thereof, prodrugs thereof, and mixtures thereof.

67.-68. (canceled)

69. A compound having the formula: 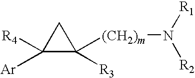
or pharmaceutically acceptable salts, esters, solvates and prodrugs thereof, where:
m is 1 or 2;
R₁ and R₂ are the same or different and are independently selected from H, or C₁-C₆-alkyl groups;
R₃ and R₄ are the same or different and are independently selected from H, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is an aryl group or heteroaryl group which can contain one or more rings at least one of which is aromatic, when the aryl or heteroaryl group contains two or more rings, the rings are either fused or optionally linked by a single bond or a linker group L, wherein L is selected from an alkyl linkage, an olefin linkage, an alkyne linkage, an ether, a thioether linkage, a ketone linkage, an amine linkage, an ester linkage, an amide linkage, a carbamyl linkage or a urea linkage with the exception that Ar is a group other than an unsubstituted phenyl group, a 4-pyridyl group, a 2-fluorophenyl group or a 4-fluorophenyl group.

70.-71. (canceled)

72. A compound of claim 69 having the formula: 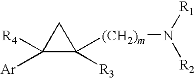
or pharmaceutically acceptable salts, esters, solvates and prodrugs thereof, where:
m is 1 or 2;
R₁ and R₂ are both hydrogens;
R₃ and R₄ are the same or different and are independently selected from hydrogen, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is an aryl group or heteroaryl group which contains two or more rings at least one of which is aromatic, when the aryl or heteroaryl group contains two or more rings, the rings are either fused or optionally linked by a single bond or a linker group L, wherein L is selected from an alkyl linkage, an olefin linkage, an alkyne linkage, an ether, a thioether linkage, a ketone linkage, an amine linkage, an ester linkage, an amide linkage, a carbamyl linkage or a urea linkage.

73. (canceled)

74. The compound of claim 69 wherein R₁ and R₂ are both hydrogen.

75. (canceled)

76. The compound of claim 69 wherein R₃ and R₄ are both hydrogens.

77. The compound of claim 69 wherein one or both of R₃ and R₄ are halogens.

78. The compound of claim 69 wherein Ar is a phenyl or a substituted phenyl which is substituted with one or more halogens, hydroxyls, alkyl groups, amide groups, ester groups, ether groups, carbamyl groups, amine groups, cyano, isocyano, nitro, haloalkyl, or hydroxyalkyl groups.

79.-80. (canceled)

81. The compound of claim 69 wherein Ar is a phenyl substituted with phenyl, a furanyl or a benzofuranyl.

82.-87. (canceled)

88. A compound having the formula: 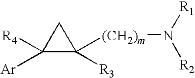
or pharmaceutically acceptable salts, esters, solvates and prodrugs thereof, where:
m is 1 or 2;
R₁ and R₂ are the same or different and are independently selected from H, or C₁-C₆-alkyl groups;
R₃ and R₄ are the same or different and are independently selected from H, halide, hydroxy, sulfhydral (—SH), nitro (—NO₂), azido (—N₃), cyano (—CN), isocyano (—NC), alkyl, alkenyl, or alkynyl, heterocyclyl, aryl, arylalkyl, heteroaryl, heteroarylalkyl, alkoxy, alkenoxy, alkynoxy, aryoxyl, heteroaryloxy, formyl, acyl, acyloxy, ether, carboxyl, oxycarbonyl, amino, alkylamino, arylamino, heteroarylamino, amido, aminoacyl, imino, carbamyl, urea, alkyl sulfide, alkenyl sulfide, alkynyl sulfide, aryl sulfide, heteroaryl sulfide, thioether, sulfonate, sulfonyl, sulfinyl, sulfonamido, silyl, phosphonate, or phosphinate group; and
Ar is a heteroaryl group linked to an aryl or to another heteroaryl group wherein the rings are either fused or optionally linked by a single bond or a linker group L, wherein L is selected from an alkyl linkage, an olefin linkage, an alkyne linkage, an ether, a thioether linkage, a ketone linkage, an amine linkage, an ester linkage, an amide linkage, a carbamyl linkage or a urea linkage.

89.-91. (canceled)

92. A compound of claim 69 selected from TKU-II-113, TKU-II-115, TKU-II-36, TKU-II-44, TKU-II-57, TKU-II-58, TKU-II-60, TKU-II-71, TKU-II-153-1 (+)-trans, TKU-II-153-2 (−)-trans, TKU-II-81, TKU-II-78, TKU-II-82, TKU-II-90, TKU-II-99, TKU-II-116, TKU-II-117, TKU-II-119, TKU-II-120, TKU-II-191, TKU-II-192, TKU-II-194, TKU-II-195, TKU-II-177, TKU-II-197, TKU-II-212, TKU-II-210, TKU-II-211, TKU-II-216, TKU-II-217, TKU-II-233, TKU-II-235, TKU-II-242, TKU-II-241 or pharmaceutically acceptable salts, esters, solvates or prodrugs thereof.

93. A compound of claim 92 selected from the (+) enantiomers of TKU-II-36, TKU-II-44, TKU-II-57, TKU-II-58, TKU-1′-60, TKU-II-71, TKU-II-81, TKU-1′-78, TKU-II-82, TKU-II-90, TKU-II-99, TKU-II-116, TKU-II-117, TKU-II-119, TKU-II-120, TKU-II-191, TKU-II-192, TKU-II-194, TKU-II-195, TKU-II-177, TKU-II-197, TKU-II-212, TKU-II-210, TKU-II-211, TKU-II-216, TKU-II-217, TKU-II-233, TKU-II-235, TKU-II-242, TKU-II-241 or pharmaceutically acceptable salts, esters, solvates or prodrugs thereof.

94. A compound of claim 93 selected from the (+) enantiomers of TKU-1144, TKU-II-78, TKU-II-90, TKU-II-57, TKU-II-58, TKU-II-81, TKU-II-82, TKU-II-119, TKU-II-120 or a pharmaceutically acceptable salt, ester, solvate, prodrug or a mixture thereof.

95. A pharmaceutical composition comprising one or more of the compounds of claim 69 in combination with a pharmaceutically acceptable carrier wherein the compound or compounds are present in the composition in a therapeutically effective amount or combined amount.

96.-114. (canceled)

115. A method for modulating a 2-HT2C receptor in vivo or in vitro which comprises contacting the receptor with one or more compounds of claim 69.
